(19) United States
(12) Patent Application Publication
(10) Pub. No.: US 2010/0144758 A1
(43) Pub. Date: Jun. 10, 2010

(54) AZAINDOLE P2X3 AND P2X2/3 MODULATORS

(76) Inventors: Michael Patrick Dillon, San Francisco, CA (US); Daisy Joe Du Bois, Menlo Park, CA (US); Yingjie Lai, Cupertino, CA (US); Ronald Charles Hawley, Glen Ridge, NJ (US); Beihan Wang, Shanghai (CN)

Correspondence Address: Grant D. Green;Patent Law Department, M/S A2-250, Roche Palo Alto LLC, 3431 Hillview Avenue, Palo Alto, CA, 94304, US

(21) Appl. No.: 12/634,031

(22) Filed: Dec. 9, 2009

Related U.S. Application Data
(60) Provisional application No. 61/201,422, filed on Dec. 9, 2008.

(51) Int. Cl. A61K 31/497; C07D 487/02; A61K 31/519; A61P 29/00; A61P 31/12; A61P 33/00; A61P 31/04; A61P 11/06; A61P 11/00

(52) U.S. Cl. 514/255.05; 544/280; 514/265.1

(57) ABSTRACT

The application discloses compounds of the Formula I:

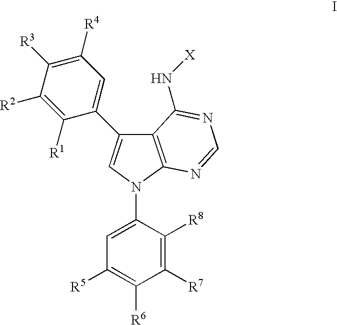

or a pharmaceutically acceptable salt thereof, wherein, X, R¹, R² R³, R⁴, R⁵, R⁶, R⁷ and R⁸ are as defined herein. Also provided are methods of using the compounds for treating diseases associated with the P2X₃ and/or a P2X_{2/3} receptor antagonist and methods of making the compounds.

### CROSS-REFERENCE TO RELATED APPLICATIONS

This application is entitled to the benefit of U.S. provisional patent application Ser. No. 61/201,422, filed on Dec. 9, 2008, the disclosure of which is incorporated herein by reference.

### FIELD OF THE INVENTION

This invention pertains to compounds useful for treatment of diseases associated with P2X purinergic receptors, and more particularly to P2X₃ and/or P2X_{2/3} antagonists usable for treatment of genitourinary, pain, gastrointestinal and respiratory diseases, conditions and disorders.

### BACKGROUND OF THE INVENTION

The urinary bladder is responsible for two important physiological functions: urine storage and urine emptying. This process involves two main steps: (1) the bladder fills progressively until the tension in its walls rises above a threshold level; and (2) a nervous reflex, called the micturition reflex, occurs that empties the bladder or, if this fails, at least causes a conscious desire to urinate. Although the micturition reflex is an autonomic spinal cord reflex, it can also be inhibited or mediated by centers in the cerebral cortex or brain.

Purines, acting via extracellular purinoreceptors, have been implicated as having a variety of physiological and pathological roles. (See, Burnstock (1993) Drug Dev. Res. 28:195-206.) ATP, and to a lesser extent, adenosine, can stimulate sensory nerve endings resulting in intense pain and a pronounced increase in sensory nerve discharge. ATP receptors have been classified into two major families, the P2Y- and P2X-purinoreceptors, on the basis of molecular structure, transduction mechanisms, and pharmacological characterization. The P2Y-purinoreceptors are G-protein coupled receptors, while the P2X-purinoreceptors are a family of ATP-gated cation channels. Purinergic receptors, in particular, P2X receptors, are known to form homomultimers or heteromultimers. To date, cDNAs for several P2X receptors subtypes have been cloned, including: six homomeric receptors, P2X₁; P2X₂; P2X₃; P2X₄; P2X₅; and P2X₇; and three heteromeric receptors P2X_{2/3}, P2X_{4/6}, P2X_{1/5} (See, e.g., Chen, et al. (1995) Nature 377:428-431; Lewis, et al. (1995) Nature 377:432-435; and Burnstock (1997) Neurophamacol. 36:1127-1139). The structure and chromosomal mapping of mouse genomic P2X₃ receptor subunit has also been described (Souslova, et al. (1997) Gene 195:101-111). In vitro, co-expression of P2X₂ and P2X₃ receptor subunits is necessary to produce ATP-gated currents with the properties seen in some sensory neurons (Lewis, et al. (1995) Nature 377:432-435).

P2X receptor subunits are found on afferents in rodent and human bladder urothelium. Data exists suggesting that ATP may be released from epithelial/endothelial cells of the urinary bladder or other hollow organs as a result of distention (Burnstock (1999) J. Anatomy 194:335-342; and Ferguson et al. (1997) J. Physiol. 505:503-511). ATP released in this manner may serve a role in conveying information to sensory neurons located in subepithelial components, e.g., suburothelial lamina propria (Namasivayam, et al. (1999) BJU Intl. 84:854-860). The P2X receptors have been studied in a number of neurons, including sensory, sympathetic, parasympathetic, mesenteric, and central neurons (Zhong, et al. (1998) Br. J. Pharmacol. 125:771-781). These studies indicate that purinergic receptors play a role in afferent neurotransmission from the bladder, and that modulators of P2X receptors are potentially useful in the treatment of bladder disorders and other genitourinary diseases or conditions.

Recent evidence also suggests a role of endogenous ATP and purinergic receptors in nociceptive responses in mice (Tsuda, et al. (1999) Br. J. Pharmacol. 128:1497-1504). ATP-induced activation of P2X receptors on dorsal root ganglion nerve terminals in the spinal cord has been shown to stimulate release of glutamate, a key neurotransmitter involved in nociceptive signaling (Gu and MacDermott, Nature 389:749-753 (1997)). P2X₃ receptors have been identified on nociceptive neurons in the tooth pulp (Cook et al., Nature 387:505-508 (1997)). ATP released from damaged cells may thus lead to pain by activating P2X₃ and/or P2X_{2/3} containing receptors on nociceptive sensory nerve endings. This is consistent with the induction of pain by intradermally applied ATP in the human blister-base model (Bleehen, Br J Pharmacol 62:573-577 (1978)). P2X antagonists have been shown to be analgesic in animal models (Driessen and Starke, Naunyn Schmiedebergs Arch Pharmaco1350:618-625 (1994)). This evidence suggests that P2X₂ and P2X₃ are involved in nociception, and that modulators of P2X receptors are potentially useful as analgesics.

Other researchers have shown that P2X₃ receptors are expressed in human colon, and are expressed at higher levels in inflamed colon than in normal colon (Y. Yiangou et al, Neurogastroenterol Mot (2001) 13:365-69). Other researchers have implicated the P2X₃ receptor in detection of distension or intraluminal pressure in the intestine, and initiation of reflex contractions (X. Bian et al., J Physiol (2003) 551.1:309-22), and have linked this to colitis (G. Wynn et al., Am J Physiol Gastrointest Liver Physiol (2004) 287:G647-57).

Inge Brouns et al. (Am J Respir Cell Mol Biol (2000) 23:52-61) found that P2X₃ receptors are expressed in pulmonary neuroepithelial bodies (NEBs), implicating the receptor in pain transmission in the lung. More recently, others have implicated P2X₂ and P2X₃ receptors in pO₂ detection in pulmonary NEBs (W. Rong et al., J Neurosci (2003) 23(36):11315-21).

There is accordingly a need for compounds that act as modulators of P2X receptors, including antagonists of P2X₃ and P2X_{2/3} receptors, as well as a need for methods of treating diseases, conditions and disorders mediated by P2X₃ and/or P2X_{2/3} receptors. The present invention satisfies these needs as well as others.

### SUMMARY OF THE INVENTION

The application provides a compound of Formula I

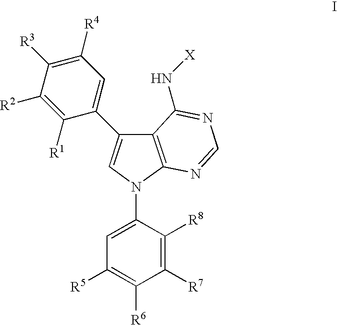

wherein:

### X is CX^{1a}X^{1b}CHX^{1c}OX^{1d};

X^{1a} and X1b are independently H, lower alkyl, or lower hydroxy alkyl;
X^{1b} is H, H lower alkyl, lower alkoxy, lower hydroxyalkyl, or hydroxy lower alkoxy; or X^{1b} and X^{1c} together form tetrahydrofuranyl;
X^{1d} is H, lower alkyl, or lower alkoxy;
   each of R¹, R², R³, and R⁴ is independently H, halogen, lower alkyl, hydroxy, lower alkoxy, amido, sulfonamido, or lower haloalkyl; and
   each of R⁵, R⁶, R⁷, and R⁸ is independently H, halogen, lower alkyl, lower alkoxy, heterocycloalkyl, or lower haloalkyl;
   with the proviso that R¹, R², R³, and R⁴ are not all H.

The application provides the compound of Formula I wherein X^{1d} is H.

The application provides the compound of Formula I wherein X^{1d} is methyl.

The application provides a compound of Formula II

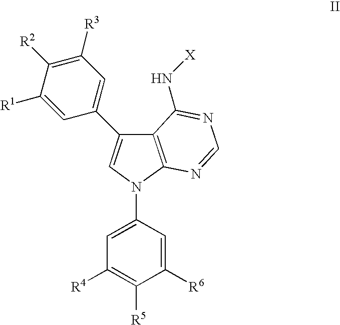

wherein:

### X is CHX^{1a}CHX^{1b}CHX^{1c}OX^{1d};

X^{1a} is H, lower alkyl, or lower hydroxy alkyl;
X^{1b} is H, H lower alkyl, lower alkoxy, lower hydroxyalkyl, or hydroxy lower alkoxy;
X^{1c} is H, lower alkyl, lower alkoxy, lower hydroxyalkyl, or hydroxy lower alkoxy;
X^{1d} is H, lower alkyl, lower alkoxy, lower haloalkyl, or lower hydroxyalkyl; or X^{1c} and X^{1d} together form tetrahydrofuranyl;
   each of R¹, R², and R³ is independently H, halogen, lower alkyl, hydroxy, lower alkoxy, amido, sulfonamido, or lower haloalkyl; and
   each of R⁴, R⁵, and R⁶ is independently H, halogen, lower alkyl, lower alkoxy, heterocycloalkyl, or lower haloalkyl;
   with the proviso that each of R¹, R², and R³ are not H.

The application provides the compound of Formula II wherein X^{1c} is H.

The application provides the compound of Formula II wherein X^{1c} is methyl.

The application provides a compound of Formula III

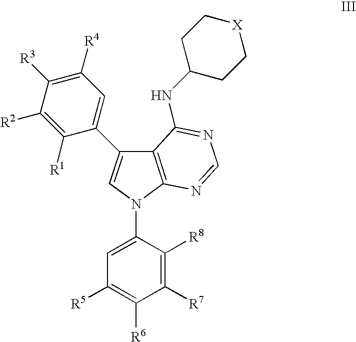

wherein:

### X is NX′ or CH(OH);

X′ is H or lower alkyl;
   each of R¹, R², R³, and R⁴ is independently H, halogen, lower alkyl, hydroxy, lower alkoxy, lower thioalkyl, amido, sulfonamido, or lower haloalkyl; and
   each of R⁵, R⁶, R⁷, and R⁸ is independently H, halogen, lower alkyl, lower alkoxy, heterocycloalkyl, or lower haloalkyl.

The application provides a compound of Formula IV

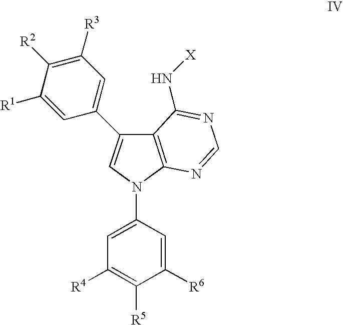

wherein:

### X is CH₂CH₂CH₂OX¹;

X¹ is H, lower alkyl, lower haloalkyl, or lower hydroxy alkyl;
   each of R¹, R², and R³ is independently H, halogen, lower alkyl, hydroxy, lower alkoxy, amido, sulfonamido, or lower haloalkyl; and
   each of R⁴, R⁵, and R⁶ is independently H, halogen, lower alkyl, lower alkoxy, heterocycloalkyl, or lower haloalkyl;
   with the proviso that when each of R¹, R², and R³ are H, X¹ is not H, methyl, ethyl or isopropyl.

The application provides a compound of Formula V

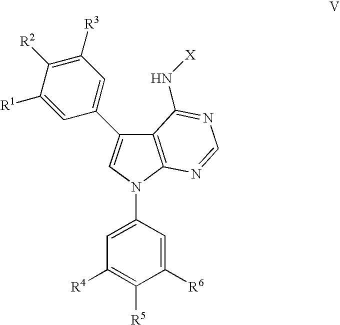

wherein:

### X is CH₂CH₂X¹;

X¹ is heterocycloalkyl or heteroaryl, optionally substituted with one or more X^{1′}; X^{1′} is lower alkyl or oxo;
   each of R¹, R², and R³ is independently H, halogen, lower alkyl, hydroxy, lower alkoxy, amido, sulfonamido, or lower haloalkyl; and
   each of R⁴, R⁵, and R⁶ is independently H, halogen, lower alkyl, lower alkoxy, heterocycloalkyl, or lower haloalkyl; and
   with the proviso that when each of R¹, R², and R³ are H, X¹ is not morpholine.

The application provides a compound of Formula VI

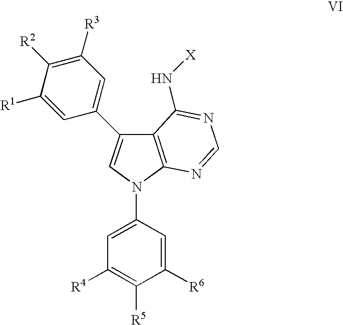

wherein:

### X is CH₂CH₂CH₂X¹;

X¹ is heterocycloalkyl or heteroaryl, optionally substituted with one or more X^{1′}; X^{1′} is lower alkyl or oxo;
   each of R¹, R², and R³ is independently H, halogen, lower alkyl, hydroxy, lower alkoxy, amido, sulfonamido, or lower haloalkyl; and
   each of R⁴, R⁵, and R⁶ is independently H, halogen, lower alkyl, lower alkoxy, heterocycloalkyl, or lower haloalkyl;
   with the proviso that when each of R¹, R², and R³ are H, X¹ is not morpholine, pyrrolidin-2-one, or imidazole.

The application provides a compound of Formula VII

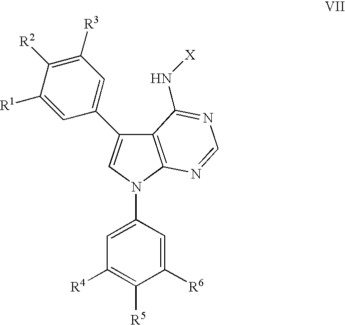

wherein:

### X is C(═O)X¹;

X¹ is lower alkyl or lower alkoxy;
   each of R¹, R², and R³ is independently H, halogen, lower alkyl, hydroxy, lower alkoxy, amido, sulfonamido, or lower haloalkyl; and
   each of R⁴, R⁵, and R⁶ is independently H, halogen, lower alkyl, lower alkoxy, heterocycloalkyl, or lower haloalkyl.

The application provides a compound of Formula VIII

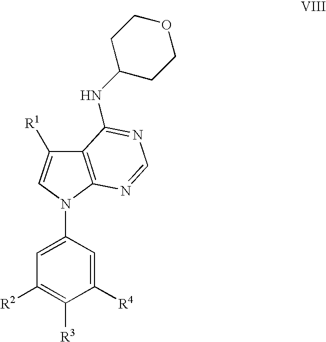

R¹ is H, halogen, hydroxy, or R′;
R′ is lower alkyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
   each of R², R³, and R⁴ is independently H, halogen, lower alkyl, lower alkoxy, heterocycloalkyl, or lower haloalkyl.

The application provides a compound of Formula IX

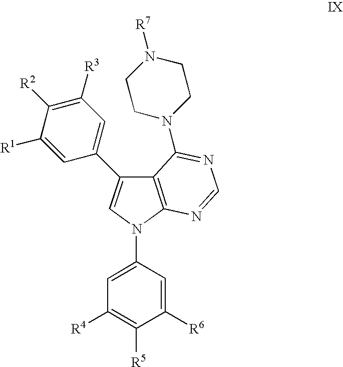

wherein:
each of R¹, R², and R³ is independently H, halogen, lower alkyl, hydroxy, lower alkoxy, amido, sulfonamido, or lower haloalkyl; and
each of R⁴, R⁵, and R⁶ is independently H, halogen, lower alkyl, lower alkoxy, heterocycloalkyl, or lower haloalkyl; and
R⁷ is lower alkyl, lower alkoxy, or lower hydroxyalkyl.

The application provides a compound of Formula X

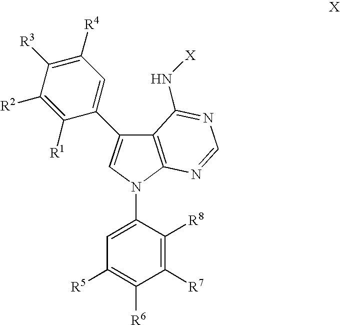

wherein:

### X is CHX^{1a}CHX^{1b}OX^{1c};

X^{1a} and X^{1b} are each independently H, lower alkyl, hydroxy, lower alkoxy, or lower hydroxy alkyl;
X^{1c} is H, lower alkyl, lower hydroxyalkyl, or lower alkoxy;
   each of R¹, R², R³, and R⁴ is independently H, halogen, lower alkyl, hydroxy, lower alkoxy, amido, sulfonamido, or lower haloalkyl; and
   each of R⁵, R⁶, R⁷, and R⁸ is independently H, halogen, lower alkyl, lower alkoxy, heterocycloalkyl, or lower haloalkyl;
   with the proviso that when each of R¹, R², R³, and R⁴ are H and X^{1c} is H, than either X^{1a} is methyl, isopropyl, or lower hydroxyalkyl;
   with the further proviso that when each of R¹, R², R³, and R⁴ are H and X^{1c} is methyl, then either R⁵ or R⁷ is fluoro, lower alkoxy, or tert-butyl; or both R⁵ and R⁷ are not H; or R⁶ is lower haloalkyl or heterocycloalkyl; or R⁷ is methyl and R⁶ is halogen or lower alkoxy; or R⁸ is fluoro; or X^{1a} is lower alkoxy or lower hydroxyalkyl.

The application provides a compound of Formula XI

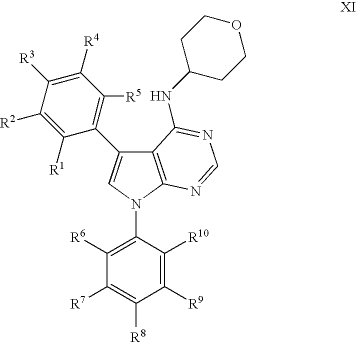

wherein:
each of R¹, R², R³, R⁴, and R⁵ is independently H, halogen, lower alkyl, lower alkenyl, hydroxy, amino, lower hydroxyalkyl, lower alkoxy, lower thioalkyl, amido, sulfonamido, lower alkyl sulfonyl, lower haloalkyl, lower haloalkoxy, cyano, or —C(═O)R′;
R′ is heterocycloalkyl, lower alkyl, lower alkoxy, hydroxy; and each of R⁶, R⁷, R⁸, R⁹, and R¹⁰ is independently H, halogen, lower alkyl, lower alkoxy, heterocycloalkyl, or lower haloalkyl.

The application provides a compound of Formula XII

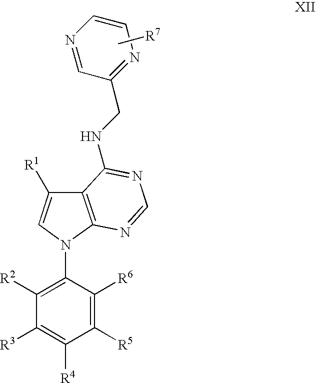

wherein:
R¹ is H, halogen, hydroxy, or R′;
R′ is lower alkyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
   each of R², R³, R⁴ R⁵, R⁶, and R⁷ is independently H, halogen, lower alkyl, lower alkoxy, heterocycloalkyl, or lower haloalkyl.

The application provides a compound of Formula XIII

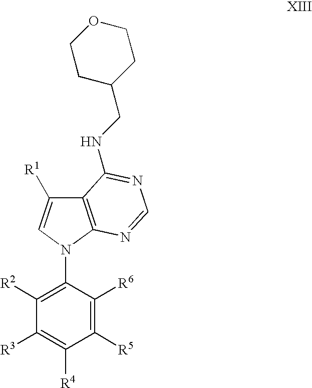

wherein:
R¹ is H, halogen, hydroxy, or R′;
R′ is lower alkyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
   each of R², R³, R⁴ R⁵, R⁶, and R⁷ is independently H, halogen, lower alkyl, lower alkoxy, heterocycloalkyl, or lower haloalkyl.

The application provides a compound of Formula XIV

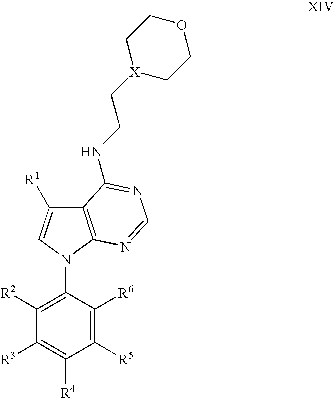

wherein:

### X is CH or N;

R¹ is H, halogen, hydroxy, or R′;
R′ is lower alkyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
   each of R², R³, R⁴ R⁵, R⁶, and R⁷ is independently H, halogen, lower alkyl, lower alkoxy, heterocycloalkyl, or lower haloalkyl.

The application provides a compound of Formula XV

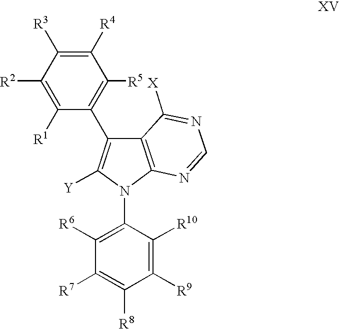

wherein:
X and Y are independently selected from H, halogen, hydroxy, and X′;
X′ is lower alkyl, phenyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more X″;
X″ is lower alkyl, halogen, hydroxy, lower alkoxy, amino, lower haloalkyl, or lower hydroxyalkyl;
R³ is halogen;
   each of R¹, R², R⁴, and R⁵ is independently H, halogen, lower alkyl, lower alkenyl, hydroxy, amino, lower hydroxyalkyl, lower alkoxy, lower thioalkyl, amido, sulfonamido, lower alkyl sulfonyl, lower haloalkyl, lower haloalkoxy, cyano, or —C(═O)R′;
R′ is heterocycloalkyl, lower alkyl, lower alkoxy, hydroxy;
   R⁷ is F, Br, I, hydroxy, or R^{7′};
R^{7′} is lower alkyl, lower alkoxy, lower haloalkyl, hydroxy, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R^{7″};
R^{7″} is lower alkyl, halogen, hydroxy, lower alkoxy, amino, lower haloalkyl, or lower hydroxyalkyl; and
   each of R⁶, R⁸, R⁹, and R¹⁹ is independently H, halogen, lower alkyl, lower alkoxy, heterocycloalkyl, or lower haloalkyl.

The application provides compounds of Formulae I-XIV selected from the group consisting of:
(5,7-Diphenyl-7H-pyrrolo[2,3-d]pyrimidin-4-y1)-(2-morpholin-4-yl-ethyl)-amine;
(2-Morpholin-4-yl-ethyl)-(5-phenyl-7-m-tolyl-7H-pyrrolo[2,3-d]pyrimidin-4-yl)-amine;
5-(4-Fluoro-phenyl)-7-m-tolyl-7H-pyrrolo[2,3-d]pyrimidin-4-ylamine;
5-(4-Fluoro-phenyl)-7-m-tolyl-7H-pyrrolo[2,3-d]pyrimidine-2,4-diamine;
5-(4-Fluoro-phenyl)-7-m-tolyl-7H-pyrrolo[2,3-d]pyrimidine;
Cyclopentyl-[5-(4-fluoro-phenyl)-7-m-tolyl-7H-pyrrolo[2,3-d]pyrimidin-4-yl]-amine;
tert-Butyl-[5-(4-fluoro-phenyl)-7-m-tolyl-7H-pyrrolo[2,3-d]pyrimidin-4-yl]-amine;
2-[5-(4-Fluoro-phenyl)-7-m-tolyl-7H-pyrrolo[2,3-d]pyrimidin-4-ylamino]-2-methyl-proan-1-ol;
5-(4-Fluoro-phenyl)-7-m-tolyl-7H-pyrrolo[2,3-d]pyrimidin-4-ol;
5-(4-Fluoro-phenyl)-4-(2-methoxy-ethoxy)-7-m-tolyl-7H-pyrrolo[2,3-d]pyrimidine;
[5-(4-Fluoro-phenyl)-7-m-tolyl-7H-pyrrolo[2,3-d]pyrimidin-4-yl]-(tetrahydro-pyran-4-yl)-amine;
(R)-2-[7-(3-Chloro-4-methyl-phenyl)-5-phenyl-7H-pyrrolo[2,3-d]pyrimidin-4-ylamino]-propan-1-ol;
2-[5-(4-Fluoro-phenyl)-7-m-tolyl-7H-pyrrolo[2,3-d]pyrimidin-4-ylamino]-2-methyl-propane-1,3-diol;
2-[7-(3-Chloro-4-methyl-phenyl)-5-phenyl-7H-pyrrolo[2,3-d]pyrimidin-4-ylamino]-propane-1,3-diol;
(2-Methoxy-ethyl)-(7-m-tolyl-5-p-tolyl-7H-pyrrolo[2,3-d]pyrimidin-4-yl)-amine;
N-[7-(3-Chloro-4-methyl-phenyl)-5-phenyl-7H-pyrrolo[2,3-d]pyrimidin-4-yl]-2-methoxy-acetamide;
[5-(4-Fluoro-phenyl)-7-m-tolyl-7H-pyrrolo[2,3-d]pyrimidin-4-yl]-(2-methoxy-ethyl)-amine;
[7-(3-Chloro-4-methyl-phenyl)-5-phenyl-7H-pyrrolo[2,3-d]pyrimidin-4-yl]-(tetrahydro-pyran-4-yl)-amine;
[5-(4-Fluoro-phenyl)-7-m-tolyl-7H-pyrrolo[2,3-d]pyrimidin-4-yl]-(2-methoxy-1-methoxymethyl-ethyl)-amine;
2-[7-(3-Chloro-4-methyl-phenyl)-5-phenyl-7H-pyrrolo[2,3-d]pyrimidin-4-ylamino]-3-methyl-butan-1-ol;
N-[7-(3-Chloro-4-methyl-phenyl)-5-phenyl-7H-pyrrolo[2,3 -d]pyrimidin-4-yl ]-propionamide;
(S)-2-[7-(3-Chloro-4-methyl-phenyl)-5-phenyl-7H-pyrrolo[2,3-d]pyrimidin-4-ylamino]-3-methyl-butan-1-ol;
2-[5-(4-Fluoro-phenyl)-7-m-tolyl-7H-pyrrolo[2,3 -d]pyrimidin-4-ylamino]-2-methyl-propan-1-ol;
(5-Pent-1-ynyl-7-m-tolyl-7H-pyrrolo[2,3-d]pyrimidin-4-yl)-(tetrahydro-pyran-4-yl)-amine;
(5-Ethynyl-7-m-tolyl-7H-pyrrolo[2,3-d]pyrimidin-4-yl)-(tetrahydro-pyran-4-yl)-amine;
(5-Pentyl-7-m-tolyl-7H-pyrrolo[2,3-d]pyrimidin-4-yl)-(tetrahydro-pyran-4-yl)-amine;
[5-(4-Fluoro -3-methyl-phenyl)-7-m-tolyl-7H-pyrrolo[2,3-d]pyrimidin-4-yl]-(tetrahydro-pyran-4-yl)-amine;
(R)-2-[7-(3-Chloro-4-methyl-phenyl)-5-phenyl-7H-pyrrolo[2,3-d]pyrimidin-4-ylamino]-3-methyl-butan-1-ol;
[5-(4-Isopropoxy-phenyl)-7-m-tolyl-7H-pyrrolo[2,3-d]pyrimidin-4-yl]-(tetrahydro-pyran-4-yl)-amine;
(5,7-Di-m-tolyl-7H-pyrrolo[2,3-d]pyrimidin-4-yl)-(2-methoxy-ethyl)-amine;
(2-M ethoxy-ethyl)-[5-(4-methoxy-phenyl)-7-m-tolyl-7H-pyrrolo[2,3-d]pyrimidin-4-yl]-amine;
(S)-2-[7-(3-Chloro-4-methyl-phenyl)-5-phenyl-7H-pyrrolo[2,3-d]pyrimidin-4-ylamino]-propan-1-ol;
4-[4-(Tetrahydro-pyran-4-ylamino)-7-m-tolyl-7H-pyrrolo[2,3-d]pyrimidin-5-yl]-phenol;
[5-(2-Chloro-phenyl)-7-m-tolyl-7H-pyrrolo[2,3-d]pyrimidin-4-yl]-(tetrahydro-pyran-4-yl)-amine;
[5-(3-Methoxy-phenyl)-7-m-tolyl-7H-pyrrolo[2,3-d]pyrimidin-4-yl]-(tetrahydro-pyran-4-yl)-amine;
[7-(3-Ethoxy-phenyl)-5-phenyl-7H-pyrrolo[2,3-d]pyrimidin-4-yl]-(2-methoxy-ethyl)-amine;
2-[5-(4-Fluoro-phenyl)-7-m-tolyl-7H-pyrrolo[2,3-d]pyrimidin-4-ylamino]-2-hydroxymethyl-propane-1,3-diol;
4-[7-(3-Chloro-4-methyl-phenyl)-5-phenyl-7H-pyrrolo[2,3-d]pyrimidin-4-ylamino]-cyclohexanol;
2-[5-(4-Fluoro-phenyl)-7-m-tolyl-7H-pyrrolo[2,3-d]pyrimidin-4-ylamino]-2-methyl-propan-1-ol;
2-[5-(4-Fluoro-phenyl)-7-m-tolyl-7H-pyrrolo[2,3-d]pyrimidin-4-ylamino]-2-methyl-propane-1,3-diol; compound with trifluoro-acetic acid;
[5-(3,5-Difluoro-phenyl)-7-m-tolyl-7H-pyrrolo[2,3-d]pyrimidin-4-yl]-(tetrahydro-pyran-4-yl)-amine;
(2-Methoxy-ethyl)-(7-m-tolyl-5-o-tolyl-7H-pyrrolo[2,3-d]pyrimidin-4-yl)-amine;
(2-Methoxy-ethyl)-[7-(3-methoxy-phenyl)-5-phenyl-7H-pyrrolo[2,3-d]pyrimidin-4-yl]-amine;
(Tetrahydro-pyran-4-yl)-(7-m-tolyl-5-p-tolyl-7H-pyrrolo[2,3-d]pyrimidin-4-yl)-amine;
[5-(3-Isopropoxy-phenyl)-7-m-tolyl-7H-pyrrolo[2,3-d]pyrimidin-4-yl]-(tetrahydro-pyran-4-yl)-amine;
[5-(4-Chloro-phenyl)-7-m-tolyl-7H-pyrrolo[2,3-d]pyrimidin-4-yl]-(2-methoxy-ethyl)-amine;
[5-(3,4-Dimethyl-phenyl)-7-m-tolyl-7H-pyrrolo[2,3-d]pyrimidin-4-yl]-(tetrahydro-pyran-4-yl)-amine;
[5-(3-Fluoro-5-methyl-phenyl)-7-m-tolyl-7H-pyrrolo[2,3-d]pyrimidin-4-yl]-(tetrahydro-pyran-4-yl)-amine;
[5-(1H-Pyrrol-2-yl)-7-m-tolyl-7H-pyrrolo[2,3-d]pyrimidin-4-yl]-(tetrahydro-pyran-4-yl)-amine;
[7-(3-tert-Butyl-phenyl)-5-phenyl-7H-pyrrolo[2,3-d]pyrimidin-4-yl]-(2-methoxy-ethyl)-amine;
(5-Methyl-7-m-tolyl-7H-pyrrolo[2,3-d]pyrimidin-4-yl)-(tetrahydro-pyran-4-yl)-amine;
[7-(3-Fluoro-phenyl)-5-phenyl-7H-pyrrolo[2,3-d]pyrimidin-4-yl]-(2-methoxy-ethyl)-amine;
[5-(4-Fluoro-phenyl)-7-m-tolyl-7H-pyrrolo[2,3-d]pyrimidin-4-yl]-(1-methyl-piperidin-4-yl)-amine;
[5-(4-Chloro-phenyl)-7-m-tolyl-7H-pyrrolo[2,3-d]pyrimidin-4-yl]-(2-methoxy-ethyl)-amine;
[5-(2,4-Difluoro-phenyl)-7-m-tolyl-7H-pyrrolo[2,3-d]pyrimidin-4-yl]-(tetrahydro-pyran-4-yl)-amine;
N-{4-[4-(Tetrahydro-pyran-4-ylamino)-7-m-tolyl-7H-pyrrolo[2,3-d]pyrimidin-5-yl]-phenyl}-acetamide;
[5-(3-Methoxy-phenyl)-7-m-tolyl-7H-pyrrolo[2,3-d]pyrimidin-4-yl]-(tetrahydro-pyran-4-yl)-amine;
(5-Pyridin-3-yl-7-m-tolyl-7H-pyrrolo[2,3-d]pyrimidin-4-yl)-(tetrahydro-pyran-4-yl)-amine;
[7-(3,5-Dichloro-phenyl)-5-phenyl-7H-pyrrolo[2,3-d]pyrimidin-4-yl]-(2-methoxy-ethyl)-amine;
[7-(4-Chloro-3-methyl-phenyl)-5-phenyl-7H-pyrrolo[2,3-d]pyrimidin-4-yl]-(2-methoxy-ethyl)-amine;
[5-(3-Chloro-5-fluoro-phenyl)-7-m-tolyl-7H-pyrrolo[2,3-d]pyrimidin-4-yl]-(tetrahydro-pyran-4-yl)-amine;
[5-(4-Fluoro-phenyl)-7-m-tolyl-7H-pyrrolo[2,3-d]pyrimidin-4-yl]-piperidin-4-yl-amine;
N-{4-[4-(Tetrahydro-pyran-4-ylamino)-7-m-tolyl-7H-pyrrolo[2,3-d]pyrimidin-5-yl]-phenyl}-methanesulfonamide;
(2-Methoxy-ethyl)-[5-phenyl-7-(4-trifluoromethyl-phenyl)-7H-pyrrolo[2,3-d]pyrimidin-4-yl]-amine;
[5-(4-Chloro-phenyl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(4-trifluoromethyl-phenyl)-7H-pyrrolo[2,3-d]pyrimidin-4-yl]-amine;
(2-Methoxy-ethyl)-[7-(4-methoxy-3-methyl-phenyl)-5-phenyl-7H-pyrrolo[2,3-d]pyrimidin-4-yl]-amine;
[5-(4-Fluoro-phenyl)-7-m-tolyl-7H-pyrrolo[2,3-d]pyrimidin-4-yl]-(2-methoxy-ethyl)-amine;
(Tetrahydro-pyran-4-yl)-(7-m-tolyl-5-o-tolyl-7H-pyrrolo[2,3-d]pyrimidin-4-yl)-amine;
4-[4-(Tetrahydro-pyran-4-ylamino)-7-m-tolyl-7H-pyrrolo[2,3-d]pyrimidin-5-yl]-benzamide;
[7-(4-Fluoro-3-methyl-phenyl)-5-phenyl-7H-pyrrolo[2,3-d]pyrimidin-4-yl]-(2-methoxy-ethyl)-amine;
2-Chloro-N,N-dimethyl-4-[4-(tetrahydro-pyran-4-ylamino)-7-m-tolyl-7H-pyrrolo[2,3-d]pyrimidin-5-yl]-benzamidep;
7-(3-Chloro-phenyl)-4-(4-methyl-piperazin-1-yl)-5-phenyl-7H-pyrrolo[2,3 -d]pyrimidine;
[5-(3,4-Dimethyl-phenyl)-7-m-tolyl-7H-pyrrolo[2,3-d]pyrimidin-4-yl]-(tetrahydro-pyran-4-yl)-amine;
2-[4-(5-Phenyl-7-m-tolyl-7H-pyrrolo[2,3-d]pyrimidin-4-yl)-piperazin-1-yl]-ethanol;
N-{3-[4-(Tetrahydro-pyran-4-ylamino)-7-m-tolyl-7H-pyrrolo[2,3-d]pyrimidin-5-yl]-phenyl}-acetamide;
[5-(2-Methylsulfanyl-phenyl)-7-m-tolyl-7H-pyrrolo[2,3-d]pyrimidin-4-yl]-(tetrahydro-pyran-4-yl)-amine;
[5-(4-Methoxy-3-trifluoromethyl-phenyl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(4-trifluoromethyl-phenyl)-7H-pyrrolo[2,3-d]pyrimidin-4-yl]-amine;
[7-(3,4-Dimethoxy-phenyl)-5-phenyl-7H-pyrrolo[2,3-d]pyrimidin-4-yl]-(2-methoxy-ethyl)-amine;
(2-M ethoxy-ethyl)-[7-(4-morpholin-4-yl-phenyl)-5-phenyl-7H-pyrrolo[2,3-d]pyrimidin-4-yl]-amine;
[5-(2-Chloro-phenyl)-7-m-tolyl-7H-pyrrolo[2,3-d]pyrimidin-4-yl]-(tetrahydro-pyran-4-yl)-amine;
2-{4-[7-(3-Chloro-phenyl)-5-phenyl-7H-pyrrolo[2,3-d]pyrimidin-4-yl]-piperazin-1yl}-ethanol;
[7-(2-Fluoro-phenyl)-5-phenyl-7H-pyrrolo[2,3-d]pyrimidin-4-yl]-(2-methoxy-ethyl)-amine;
[7-(3-Chloro-4-methyl-phenyl)-5-phenyl-7H-pyrrolo[2,3-d]pyrimidin-4-yl]-(2-methoxy-1-methoxymethyl-ethyl)-amine;
[7-(3-Chloro-4-methyl-phenyl)-5-phenyl-7H-pyrrolo[2,3-d]pyrimidin-4-yl]-(2-methoxy-1-methoxymethyl-ethyl)-amine;
[7-(3-Chloro-4-methyl-phenyl)-5-phenyl-7H-pyrrolo[2,3-d]pyrimidin-4-yl]-(2-pyridin-3-yl-ethyl)-amine;
[7-(3-Chloro-4-methyl-phenyl)-5-phenyl-7H-pyrrolo[2,3-d]pyrimidin-4-yl]-(2-pyridin-2-yl-ethyl)-amine;
2-Chloro-4-[4-(tetrahydro-pyran-4-ylamino)-7-m-tolyl-7H-pyrrolo[2,3-d]pyrimidin-5-yl]-benzamide
[7-(3-Chloro-4-methyl-phenyl)-5-phenyl-7H-pyrrolo[2,3-d]pyrimidin-4-yl]-(2-imidazol-1-yl-ethyl)-amine;
[7-(3-Fluoro-4-methyl-phenyl)-5-phenyl-7H-pyrrolo[2,3-d]pyrimidin-4-yl]-(2-methoxy-ethyl)-amine;
4-(4-Methyl-piperazin-1-yl)-5-phenyl-7-p-tolyl-7H-pyrrolo[2,3-d]pyrimidine;
{2-Chloro-4-[4-(tetrahydro-pyran-4-ylamino)-7-m-tolyl-7H-pyrrolo[2,3-d]pyrimidin-5-yl]-phenyl}-pyrrolidin-1-yl-methanone;
{2-Chloro-4-[4-(tetrahydro-pyran-4-ylamino)-7-m-tolyl-7H-pyrrolo[2,3-d]pyrimidin-5-yl]-phenyl}-morpholin-4-yl-methanone;
2-Fluoro-4-[4-(tetrahydro-pyran-4-ylamino)-7-m-tolyl-7H-pyrrolo[2,3-d]pyrimidin-5-yl]-benzoic acid methyl ester;
[5-(5-Chloro-2-methyl-phenyl)-7-m-tolyl-7H-pyrrolo[2,3-d]pyrimidin-4-yl]-(tetrahydro-pyran-4-yl)-amine;
[5-(2,3-Difluoro-phenyl)-7-m-tolyl-7H-pyrrolo[2,3-d]pyrimidin-4-yl]-(tetrahydro-pyran-4-yl)-amine;
[5-(3-Fluoro-phenyl)-7-m-tolyl-7H-pyrrolo[2,3-d]pyrimidin-4-yl]-(tetrahydro-pyran-4-yl)-amine;
[5-(3-Ethoxy-phenyl)-7-m-tolyl-7H-pyrrolo[2,3-d]pyrimidin-4-yl]-(tetrahydro-pyran-4-yl)-amine;
[5-(4-Methylsulfanyl-phenyl)-7-m-tolyl-7H-pyrrolo[2,3-d]pyrimidin-4-yl]-(tetrahydro-pyran-4-yl)-amine;
[5-(2-Ethoxy-phenyl)-7-m-tolyl-7H-pyrrolo[2,3-d]pyrimidin-4-yl]-(tetrahydro-pyran-4-yl)-amine;
[5-(3,5-Dimethyl-phenyl)-7-m-tolyl-7H-pyrrolo[2,3-d]pyrimidin-4-yl]-(tetrahydro-pyran-4-yl)-amine;
[5-(4-Fluoro-3-trifluoromethyl-phenyl)-7-m-tolyl-7H-pyrrolo[2,3-d]pyrimidin-4-yl]-(tetrahydro-pyran-4-yl)-amine;
[5-(3-Fluoro-4-methoxy-phenyl)-7-m-tolyl-7H-pyrrolo[2,3-d]pyrimidin-4-yl]-(tetrahydro-pyran-4-yl)-amine;
[5-(3-Fluoro-5-isopropoxy-phenyl)-7-m-tolyl-7H-pyrrolo[2,3-d]pyrimidin-4-yl]-(tetrahydro-pyran-4-yl)-amine;
[5-(3-Ethoxy-5-fluoro-phenyl)-7-m-tolyl-7H-pyrrolo[2,3-d]pyrimidin-4-yl]-(tetrahydro-pyran-4-yl)-amine;
[5-(3-Methylsulfanyl-phenyl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(3-trifluoromethyl-phenyl)-7H-pyrrolo[2,3-d]pyrimidin-4-yl]-amine;
(Tetrahydro-pyran-4-yl)-[7-m-tolyl-5-(3-trifluoromethyl-phenyl)-7H-pyrrolo[2,3-d]pyrimidin-4-yl]-amine;
[5-(4-tert-Butyl-phenyl)-7-m-tolyl-7H-pyrrolo[2,3-d]pyrimidin-4-yl]-(tetrahydro-pyran-4-yl)-amine;
[5-(2-Ethyl-phenyl)-7-m-tolyl-7H-pyrrolo[2,3-d]pyrimidin-4-yl]-(tetrahydro-pyran-4-yl)-amine;
[5-(2-Ethyl-phenyl)-7-m-tolyl-7H-pyrrolo[2,3-d]pyrimidin-4-yl]-(tetrahydro-pyran-4-yl)-amine;
[5-(2-Fluoro-phenyl)-7-m-tolyl-7H-pyrrolo[2,3-d]pyrimidin-4-yl]-(tetrahydro-pyran-4-yl)-amine;
[5-(2-Methoxy-phenyl)-7-m-tolyl-7H-pyrrolo[2,3-d]pyrimidin-4-yl]-(tetrahydro-pyran-4-yl)-amine;
[5-(4-Ethyl-phenyl)-7-m-tolyl-7H-pyrrolo[2,3-d]pyrimidin-4-yl]-(tetrahydro-pyran-4-yl)-amine;
3-[4-(Tetrahydro-pyran-4-ylamino)-7-m-tolyl-7H-pyrrolo[2,3-d]pyrimidin-5-yl]-benzonitrile;
[5-(4-Ethoxy-phenyl)-7-m-tolyl-7H-pyrrolo[2,3-d]pyrimidin-4-yl]-(tetrahydro-pyran-4-yl)-amine;
[5-(3-Chloro-phenyl)-7-m-tolyl-7H-pyrrolo[2,3-d]pyrimidin-4-yl]-(tetrahydro-pyran-4-yl)-amine;
[5-(3,4-Dimethoxy-phenyl)-7-m-tolyl-7H-pyrrolo[2,3-d]pyrimidin-4-yl]-(tetrahydro-pyran-4-yl)-amine;
[5-(3-Isopropyl-phenyl)-7-m-tolyl-7H-pyrrolo[2,3-d]pyrimidin-4-yl]-(tetrahydro-pyran-4-yl)-amine;
[5-(4-Isopropyl-phenyl)-7-m-tolyl-7H-pyrrolo[2,3-d]pyrimidin-4-yl]-(tetrahydro-pyran-4-yl)-amine;
(5,7-Di-m-tolyl-7H-pyrrolo[2,3-d]pyrimidin-4-yl)-(tetrahydro -pyran-4-yl)-amine
[5-(4-Methoxy-phenyl)-7-m-tolyl-7H-pyrrolo[2,3-d]pyrimidin-4-yl]-(tetrahydro-pyran-4-yl)-amine;
[5-(2-Isopropoxy-phenyl)-7-m-tolyl-7H-pyrrolo[2,3-d]pyrimidin-4-yl]-(tetrahydro-pyran-4-yl)-amine;
(5-Ethyl-7-m-tolyl-7H-pyrrolo[2,3-d]pyrimidin-4-yl)-(tetrahydro -pyran-4-yl)-amine;
(Tetrahydro-pyran-4-yl)-[7-m-tolyl-5-(2-p-tolyl-ethyl)-7H-pyrrolo[2,3-d]pyrimidin-4-yl]-amine;
(5-Butyl-7-m-tolyl-7H-pyrrolo[2,3-d]pyrimidin-4-yl)-(tetrahydro-pyran-4-yl)-amine;
(Tetrahydro-pyran-4-yl)-[7-m-tolyl-5-(3,4,5-trimethoxy-phenyl)-7H-pyrrolo[2,3-d]pyrimidin-4-yl]-amine
[5-(4-Methanesulfonyl-phenyl)-7-m-tolyl-7H-pyrrolo[2,3-d]pyrimidin-4-yl]-(tetrahydro-pyran-4-yl)-amine;
1-{4-[4-(Tetrahydro-pyran-4-ylamino)-7-m-tolyl-7H-pyrrolo[2,3-d]pyrimidin-5-yl]-phenyl}-ethanone;
1-{2-[4-(Tetrahydro-pyran-4-ylamino)-7-m-tolyl-7H-pyrrolo[2,3-d]pyrimidin-5-yl]-phenyl}-ethanone;
4-[4-(Tetrahydro-pyran-4-ylamino)-7-m-tolyl-7H-pyrrolo[2,3-d]pyrimidin-5-yl]-benzoic acid ethyl ester;
(Tetrahydro-pyran-4-yl)-(5-thiophen-2-yl-7-m-tolyl-7H-pyrrolo[2,3-d]pyrimidin-4-yl)-amine;
N-{3-[4-(Tetrahydro-pyran-4-ylamino)-7-m-tolyl-7H-pyrrolo[2,3-d]pyrimidin-5-yl]-phenyl}-methanesulfonamide;
(5-Quinolin-8-yl-7-m-tolyl-7H-pyrrolo[2,3-d]pyrimidin-4-yl)-(tetrahydro-pyran-4-yl)-amine;
N-Methyl-4-[4-(tetrahydro-pyran-4-ylamino)-7-m-tolyl-7H-pyrrolo[2,3-d]pyrimidin-5-yl]-benzenesulfonamide;
[5-(3-Amino-phenyl)-7-m-tolyl-7H-pyrrolo[2,3-d]pyrimidin-4-yl]-(tetrahydro-pyran-4-yl)-amine;
N,N-Diethyl-2-[4-(tetrahydro-pyran-4-ylamino)-7-m-tolyl-7H-pyrrolo[2,3-d]pyrimidin-5-yl]-benzamide;
[5-((E)-Pent-1-enyl)-7-m-tolyl-7H-pyrrolo[2,3-d]pyrimidin-4-yl]-(tetrahydro-pyran-4-yl)-amine;
[5-(1H-Pyrrol-3-yl)-7-m-tolyl-7H-pyrrolo[2,3-d]pyrimidin-4-yl]-(tetrahydro-pyran-4-yl)-amine;
[5-(1-Methyl-1H-pyrrol-2-yl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(2-vinyl-phenyl)-7H-pyrrolo[2,3-d]pyrimidin-4-yl]-amine;
N-tert-Butyl-3-[4-(tetrahydro-pyran-4-ylamino)-7-m-tolyl-7H-pyrrolo[2,3-d]pyrimidin-5-yl]-benzenesulfonamide;
(5-Cyclopent-1-enyl-7-m-tolyl-7H-pyrrolo[2,3-d]pyrimidin-4-yl)-(tetrahydro-pyran-4-yl)-amine;
(5-Cyclohex-1-enyl-7-m-tolyl-7H-pyrrolo[2,3-d]pyrimidin-4-yl)-(tetrahydro-pyran-4-yl)-amine;
3-[4-(Tetrahydro-pyran-4-ylamino)-7-m-tolyl-7H-pyrrolo[2,3-d]pyrimidin-5-yl]-benzenesulfonamide;
[5-(4-Dimethylamino-phenyl)-7-m-tolyl-7H-pyrrolo[2,3-d]pyrimidin-4-yl]-(tetrahydro-pyran-4-yl)-amine;
[5-(3-Chloro-4-fluoro-phenyl)-7-m-tolyl-7H-pyrrolo[2,3-d]pyrimidin-4-yl]-(tetrahydro-pyran-4-yl)-amine;
(Tetrahydro-pyran-4-yl)-(5-thiophen-3-yl-7-m-tolyl-7H-pyrrolo[2,3-d]pyrimidin-4yl)-amine;
1-{5-[4-(Tetrahydro-pyran-4-ylamino)-7-m-tolyl-7H-pyrrolo[2,3-d]pyrimidin-5-yl]-thiophen-2-yl}-ethanone;
[5-(4-Methyl-thiophen-2-yl)-7-m-tolyl-7H-pyrrolo[2,3-d]pyrimidin-4-yl]-(tetrahydro-pyran-4-yl)-amine;
[5-(3,4-Difluoro-phenyl)-7-m-tolyl-7H-pyrrolo[2,3-d]pyrimidin-4-yl]-(tetrahydro-pyran-4-yl)-amine;
(5-Phenyl-7-m-tolyl-7H-pyrrolo[2,3-d]pyrimidin-4-yl)-(tetrahydro-pyran-4-yl)-amine;
(5-Phenyl-7-m-tolyl-7H-pyrrolo[2,3-d]pyrimidin-4-yl)-[2-(tetrahydro-pyran-4-yl)-ethyl]-amine;
(5-Phenyl-7-m-tolyl-7H-pyrrolo[2,3-d]pyrimidin-4-yl)-(tetrahydro-pyran-4-ylmethyl)-amine;
(5,7-Diphenyl-7H-pyrrolo[2,3-d]pyrimidin-4-yl)-(tetrahydro-pyran-4-yl)-amine;
(5,7-Diphenyl-7H-pyrrolo[2,3-d]pyrimidin-4-yl)-(tetrahydro-pyran-4-ylmethyl)-amine;
[5-(1-Methyl-1H-pyrazol-4-yl)-7-m-tolyl-7H-pyrrolo[2,3-d]pyrimidin-4-yl]-(tetrahydro-pyran-4-yl)-amine;
[5-(1H-Pyrazol-3-yl)-7-m-tolyl-7H-pyrrolo[2,3-d]pyrimidin-4-yl]-(tetrahydro-pyran-4-yl)-amine;
[5-(1H-Pyrazol-4-yl)-7-m-tolyl-7H-pyrrolo[2,3-d]pyrimidin-4-yl]-(tetrahydro-pyran-4-yl)-amine;
(S)-2-[5-(2-Methyl-propenyl)-7-p-tolyl-7H-pyrrolo[2,3-d]pyrimidin-4-ylamino]-propan-1-ol; and
(S)-2-(5-Isobutyl-7-p-tolyl-7H-pyrrolo[2,3-d]pyrimidin-4-ylamino)-propan-1-ol.

The application provides a pharmaceutical composition comprising:
(a) a pharmaceutically acceptable carrier; and
(b) the compound of any one of Formulae I-XV.

The application provides a method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof an effective amount of the compound of any one of Formulae I-XV.

The application provides a method for treating a respiratory disorder selected from chronic obstructive pulmonary disorder (COPD), asthma, and bronchospasm, said method comprising administering to a subject in need thereof an effective amount of the compound of any one of Formulae I-XV.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Agonist” refers to a compound that enhances the activity of another compound or receptor site.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkynyl” means a linear monovalent hydrocarbon radical of two to six carbon atoms or a branched monovalent hydrocarbon radical of three to six carbon atoms, containing at least one triple bond, e.g., ethynyl, propynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Alkoxyalkyl” means a moiety of the formula R^{a}—O—R^{b}—, where R^{a} is alkyl and R^{b} is alkylene as defined herein. Exemplary alkoxyalkyl groups include, by way of example, 2-methoxyethyl, 3-methoxypropyl, 1-methyl-2-methoxyethyl, 1-(2-methoxyethyl)-3-methoxypropyl, and 1-(2-methoxyethyl)-3-methoxypropyl.

“Alkylcarbonyl” means a moiety of the formula —R′—R″, where R′ is oxo and R″ is alkyl as defined herein.

“Alkylsulfonyl” means a moiety of the formula —R′—R″, where R′ is —SO₂— and R″ is alkyl as defined herein.

“Alkylsulfonylalkyl means a moiety of the formula —R′—R″—R′″ where where R′ is alkylene, R″ is —SO₂— and R′″ is alkyl as defined herein.

“Alkylamino means a moiety of the formula —NR—R′ wherein R is hyrdogen or alkyl and R′ is alkyl as defined herein.

“Alkoxyamino” means a moiety of the formula —NR—OR′ wherein R is hydrogen or alkyl and R′ is alkyl as defined herein.

“Alkylsulfanyl” means a moiety of the formula —SR wherein R is alkyl as defined herein.

“Aminoalkyl” means a group —R—R′ wherein R′ is amino and R is alkylene as defined herein. “Aminoalkyl” includes aminomethyl, aminoethyl, 1-aminopropyl, 2-aminopropyl, and the like. The amino moiety of “aminoalkyl” may be substituted once or twice with alkyl to provide “alkylaminoalkyl” and “dialkylaminoalkyl” respectively. “Alkylaminoalkyl” includes methylaminomethyl, methylaminoethyl, methylaminopropyl, ethylaminoethyl and the like. “Dialkylamino alkyl” includes dimethylaminomethyl, dimethylaminoethyl, dimethylaminopropyl, N-methyl-N-ethylaminoethyl, and the like.

“Aminoalkoxy” means a group —OR—R′ wherein R′ is amino and R is alkylene as defined herein.

“Alkylsulfonylamido” means a moiety of the formula —NR′SO₂—R wherein R is alkyl and R′ is hydrogen or alkyl.

“Aminocarbonyloxyalkyl” or “carbamylalkyl” means a group of the formula —R—O—C(O)—NR′R″ wherein R is alkylene and R′, R″ each independently is hydrogen or alkyl as defined herein.

“Alkynylalkoxy” means a group of the formula —O—R—R′ wherein R is alkylene and R′ is alkynyl as defined herein.

“Antagonist” refers to a compound that diminishes or prevents the action of another compound or receptor site.

“Aryl” means a monovalent cyclic aromatic hydrocarbon moiety consisting of a mono-, bi- or tricyclic aromatic ring. The aryl group can be optionally substituted as defined herein. Examples of aryl moieties include, but are not limited to, optionally substituted phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof.

“Arylalkyl” and “Aralkyl”, which may be used interchangeably, mean a radical-R^{a}R^{b} where R^{a} is an alkylene group and R^{b} is an aryl group as defined herein; e.g., phenylalkyls such as benzyl, phenylethyl, 3-(3-chlorophenyl)-2-methylpentyl, and the like are examples of arylalkyl.

“Arylsulfonyl means a group of the formula —SO₂—R wherein R is aryl as defined herein.

“Aryloxy” means a group of the formula —O—R wherein R is aryl as defined herein.

“Aralkyloxy” means a group of the formula —O—R—R″ wherein R is alkylene and R′ is aryl as defined herein.

“Cyanoalkyl” ” means a moiety of the formula —R′—R″, where R′ is alkylene as defined herein and R″ is cyano or nitrile.

“Cycloalkyl” means a monovalent saturated carbocyclic moiety consisting of mono- or bicyclic rings. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated derivatives thereof.

“Cycloalkylalkyl” means a moiety of the formula —R′—R″, where R′ is alkylene and R″ is cycloalkyl as defined herein.

“Heteroaryl” means a monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof

“Heteroarylalkyl” or “heteroaralkyl” means a group of the formula —R—R′ wherein R is alkylene and R′ is heteroaryl as defined herein.

The terms “halo”, “halogen” and “halide”, which may be used interchangeably, refer to a substituent fluoro, chloro, bromo, or iodo.

“Haloalkyl” or “lower haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Haloalkoxy” means a moiety of the formula —OR, wherein R is a haloalkyl moiety as defined herein. An exemplary haloalkoxy is difluoromethoxy.

“Heterocyclyl” or “heterocycloalkyl” means a monovalent saturated moiety, consisting of one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, homopiperazinyl, azepinyl, pyrrolidinyl, pyrazolidinyl, imidazolinyl, imidazolidinyl, pyridinyl, pyridazinyl, pyrimidinyl, oxazolidinyl, isoxazolidinyl, morpholinyl, thiazolidinyl, isothiazolidinyl, quinuclidinyl, quinolinyl, isoquinolinyl, benzimidazolyl, thiadiazolylidinyl, benzothiazolidinyl, benzoazolylidinyl, dihydrofuryl, tetrahydrofuryl, dihydropyranyl, tetrahydropyranyl, thiamorpholinyl, thiamorpholinylsulfoxide, thiamorpholinylsulfone, dihydroquinolinyl, dihydrisoquinolinyl, tetrahydroquinolinyl, tetrahydrisoquinolinyl, and the like.

“Hydroxyalkyl” means a moiety of the formula —ROH wherein R is alkyl as defined herein.

“Hydroxyalkoxy” means a moiety of the formula —OR wherein R is hydroxyalkyl as defined herein.

“Urea”or “ureido” means a group of the formula —NR′—C(O)—NR″R′″ wherein R′, R″ and R′″ each independently is hydrogen or alkyl.

“Carbamate” means a group of the formula —O—C(O)—NR′R″ wherein R′ and R″ each independently is hydrogen or alkyl.

“Carboxy” means a group of the formula —O—C(O)—OH.

“Sulfonamido” means a group of the formula —SO₂—NR′R″ wherein R′, R″ and R′″ each independently is hydrogen or alkyl.

“Optionally substituted”, when used in association with “aryl”, phenyl”, “heteroaryl” “cycloalkyl” or “heterocyclyl”, means an aryl, phenyl, heteroaryl, cyclohexyl or heterocyclyl which is optionally substituted independently with one to four substituents, preferably one or two substituents selected from alkyl, cycloalkyl, cycloalkylalkyl, heteroalkyl, hydroxyalkyl, halo, nitro, cyano, hydroxy, alkoxy, amino, acylamino, mono-alkylamino, di-alkylamino, haloalkyl, haloalkoxy, heteroalkyl, —COR (where R is hydrogen, alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{a}R^{b} (where n is an integer from 0 to 5, R′ and R″ are independently hydrogen or alkyl, and R^{a} and R^{b} are, independently of each other, hydrogen, alkyl, cycloalkyl, cycloalkylalkyl, phenyl or phenylalkyl). Certain preferred optional substituents for “aryl”, phenyl”, “heteroaryl” “cycloalkyl” or “heterocyclyl” include alkyl, halo, haloalkyl, alkoxy, cyano, amino and alkylsulfonyl. More preferred substituents are methyl, fluoro, chloro, trifluoromethyl, methoxy, amino and methanesulfonyl.

“Leaving group” means the group with the meaning conventionally associated with it in synthetic organic chemistry, i.e., an atom or group displaceable under substitution reaction conditions. Examples of leaving groups include, but are not limited to, halogen, alkane- or arylenesulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Inert organic solvent” or “inert solvent” means the solvent is inert under the conditions of the reaction being described in conjunction therewith, including for example, benzene, toluene, acetonitrile, tetrahydrofuran, N,N-dimethylformamide, chloroform, methylene chloride or dichloromethane, dichloroethane, diethyl ether, ethyl acetate, acetone, methyl ethyl ketone, methanol, ethanol, propanol, isopropanol, tent-butanol, dioxane, pyridine, and the like. Unless specified to the contrary, the solvents used in the reactions of the present invention are inert solvents.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:
acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or
salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Protective group” or “protecting group” means the group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive nitrogen and/or oxygen atoms present in the reactants. For example, the terms “amino-protecting group” and “nitrogen protecting group” are used interchangeably herein and refer to those organic groups intended to protect the nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), and the like. The artisan in the art will know how to chose a group for the ease of removal and for the ability to withstand the following reactions.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Disorders of the urinary tract” or “uropathy” used interchangeably with “symptoms of the urinary tract” means the pathologic changes in the urinary tract. Examples of urinary tract disorders include, but are not limited to, incontinence, benign prostatic hypertrophy (BPH), prostatitis, detrusor hyperreflexia, outlet obstruction, urinary frequency, nocturia, urinary urgency, overactive bladder, pelvic hypersensitivity, urge incontinence, urethritis, prostatodynia, cystitis, idiophatic bladder hypersensitivity, and the like.

“Disease states associated with the urinary tract” or “urinary tract disease states” or “uropathy” used interchangeably with “symptoms of the urinary tract” mean the pathologic changes in the urinary tract, or dysfunction of urinary bladder smooth muscle or its innervation causing disordered urinary storage or voiding. Symptoms of the urinary tract include, but are not limited to, overactive bladder (also known as detrusor hyperactivity), outlet obstruction, outlet insufficiency, and pelvic hypersensitivity.

“Overactive bladder” or “detrusor hyperactivity” includes, but is not limited to, the changes symptomatically manifested as urgency, frequency, altered bladder capacity, incontinence, micturition threshold, unstable bladder contractions, sphincteric spasticity, detrusor hyperreflexia (neurogenic bladder), detrusor instability, and the like.

“Outlet obstruction” includes, but is not limited to, benign prostatic hypertrophy (BPH), urethral stricture disease, tumors, low flow rates, difficulty in initiating urination, urgency, suprapubic pain, and the like.

“Outlet insufficiency” includes, but is not limited to, urethral hypermobility, intrinsic sphincteric deficiency, mixed incontinence, stress incontinence, and the like.

“Pelvic Hypersensitivity” includes, but is not limited to, pelvic pain, interstitial (cell) cystitis, prostatodynia, prostatitis, vulvadynia, urethritis, orchidalgia, overactive bladder, and the like.

“Respiratory disorder” refers to, without limitation, chronic obstructive pulmonary disease (COPD), asthma, bronchospasm, and the like.

“Gastrointestinal disorder” (“GI disorder”) refers to, without limitation, Irritable Bowel Syndrome (IBS), Inflammatory Bowel Disease (IBD), biliary colic and other biliary disorders, renal colic, diarrhea-dominant IBS, pain associated with GI distension, and the like.

“Pain” includes, without limitation, inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes:
(i) preventing the disease state, i.e. causing the clinical symptoms of the disease state not to develop in a subject that may be exposed to or predisposed to the disease state, but does not yet experience or display symptoms of the disease state.
(ii) inhibiting the disease state, i.e., arresting the development of the disease state or its clinical symptoms, or
(iii) relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Nomenclature and Structures

In general, the nomenclature used in this Application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen or nitrogen atom in the structures herein indicates the presence of a hydrogen atom. Where a chiral center exists in a structure but no specific stereochemistry is shown for the chiral center, both enantiomers associated with the chiral structure are encompassed by the structure.

All patents and publications identified herein are incorporated herein by reference in their entirety.

Representative compounds of the invention are shown in Table 1 below.

TABLE 1

| # | Structure | Name (Autonom ™) |
|---|---|---|
| 1 | 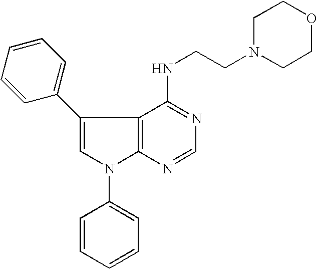 | (5,7-Diphenyl-7H-pyrrolo[2,3- d]pyrimidin-4-yl)-(2-morpholin-4-yl- ethyl)-amine |
| 2 | 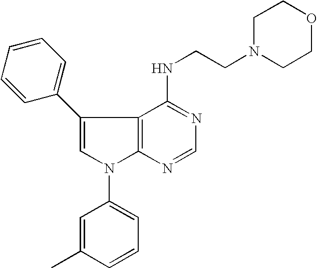 | (2-Morpholin-4-yl-ethyl)-(5-phenyl-7- m-tolyl-7H-pyrrolo[2,3-d]pyrimidin-4- yl)-amine |
| 3 | 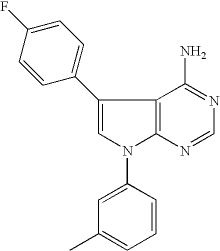 | 5-(4-Fluoro-phenyl)-7-m-tolyl-7H- pyrrolo[2,3-d]pyrimidin-4-ylamine |
| 4 | 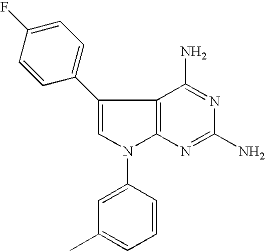 | 5-(4-Fluoro-phenyl)-7-m-tolyl-7H- pyrrolo[2,3-d]pyrimidine-2,4-diamine |
| 5 | 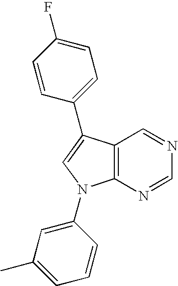 | 5-(4-Fluoro-phenyl)-7-m-tolyl-7H- pyrrolo[2,3-d]pyrimidine |
| 6 | 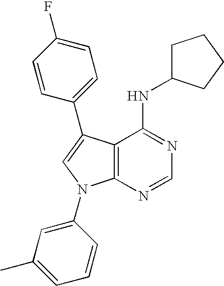 | Cyclopentyl-[5-(4-fluoro-phenyl)-7-m- tolyl-7H-pyrrolo[2,3-d]pyrimidin-4-yl]- amine |
| 7 | 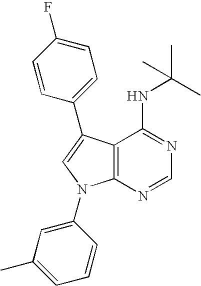 | tert-Butyl-[5-(4-fluoro-phenyl)-7-m- tolyl-7H-pyrrolo[2,3-d]pyrimidin-4-yl]- amine |
| 8 | 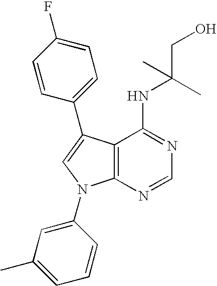 | 2-[5-(4-Fluoro-phenyl)-7-m-tolyl-7H- pyrrolo[2,3-d]pyrimidin-4-ylamino]-2- methyl-propan-1-ol |
| 9 | 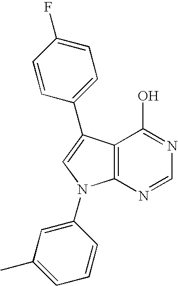 | 5-(4-Fluoro-phenyl)-7-m-tolyl-7H- pyrrolo[2,3-d]pyrimidin-4-ol |
| 10 | 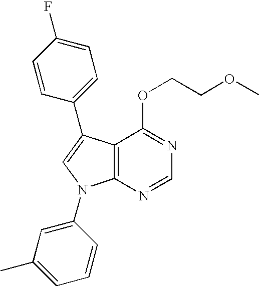 | 5-(4-Fluoro-phenyl)-4-(2-methoxy- ethoxy)-7-m-tolyl-7H-pyrrolo[2,3- d]pyrimidine |
| 11 | 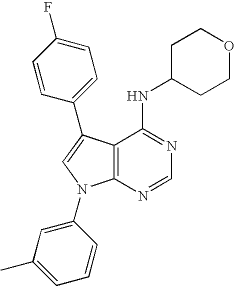 | [5-(4-Fluoro-phenyl)-7-m-tolyl-7H- pyrrolo[2,3-d]pyrimidin-4-yl]- (tetrahydro-pyran-4-yl)-amine |
| 12 | 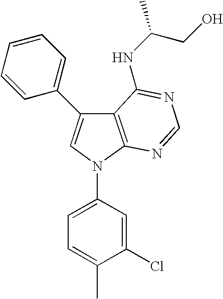 | (R)-2-[7-(3-Chloro-4-methyl-phenyl)- 5-phenyl-7H-pyrrolo[2,3-d]pyrimidin- 4-ylamino]-propan-1-ol |
| 13 | 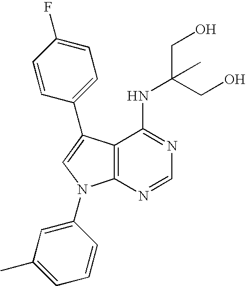 | 2-[5-(4-Fluoro-phenyl)-7-m-tolyl-7H- pyrrolo[2,3-d]pyrimidin-4-ylamino]-2- methyl-propane-1,3-diol |
| 14 | 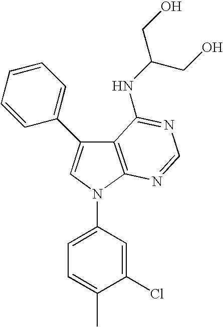 | 2-[7-(3-Chloro-4-methyl-phenyl)-5- phenyl-7H-pyrrolo[2,3-d]pyrimidin-4- ylamino]-propane-1,3-diol |
| 15 | 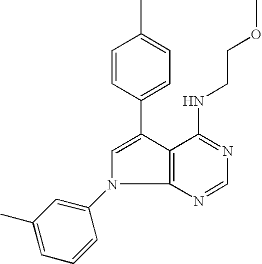 | (2-Methoxy-ethyl)-(7-m-tolyl-5-p- tolyl-7H-pyrrolo[2,3-d]pyrimidin-4-yl)- amine |
| 16 | 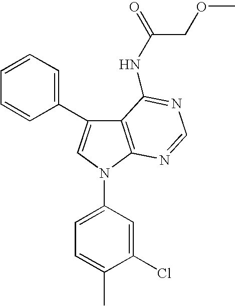 | N-[7-(3-Chloro-4-methyl-phenyl)-5- phenyl-7H-pyrrolo[2,3-d]pyrimidin-4- yl]-2-methoxy-acetamide |
| 17 | 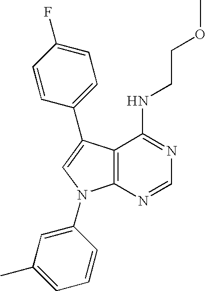 | [5-(4-Fluoro-phenyl)-7-m-tolyl-7H- pyrrolo[2,3-d]pyrimidin-4-yl]-(2- methoxy-ethyl)-amine |
| 18 | 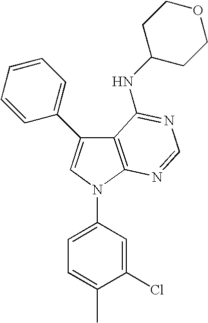 | [7-(3-Chloro-4-methyl-phenyl)-5- phenyl-7H-pyrrolo[2,3-d]pyrimidin-4- yl]-(tetrahydro-pyran-4-yl)-amine |
| 19 | 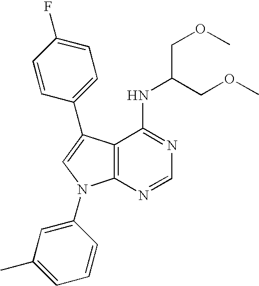 | [5-(4-Fluoro-phenyl)-7-m-tolyl-7H- pyrrolo[2,3-d]pyrimidin-4-yl]-(2- methoxy-1-methoxymethyl-ethyl)- amine |
| 20 | 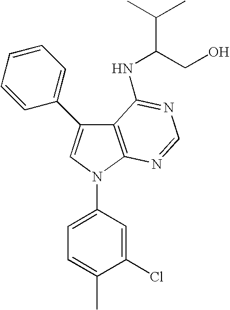 | 2-[7-(3-Chloro-4-methyl-phenyl)-5- phenyl-7H-pyrrolo[2,3-d]pyrimidin-4- ylamino]-3-methyl-butan-1-ol |
| 21 | 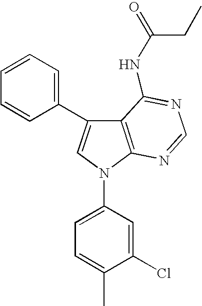 | N-[7-(3-Chloro-4-methyl-phenyl)-5- phenyl-7H-pyrrolo[2,3-d]pyrimidin-4- yl]-propionamide |
| 22 | 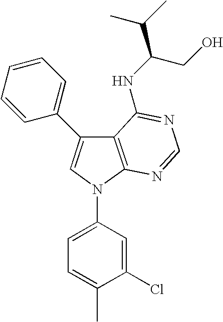 | (S)-2-[7-(3-Chloro-4-methyl-phenyl)-5- phenyl-7H-pyrrolo[2,3-d]pyrimidin-4- ylamino]-3-methyl-butan-1-ol |
| 23 | 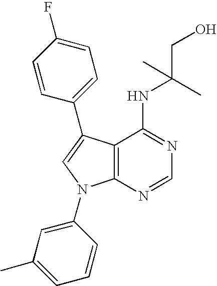 | 2-[5-(4-Fluoro-phenyl)-7-m-tolyl-7H- pyrrolo[2,3-d]pyrimdiin-4-ylamino]-2- methyl-propan-1-ol |
| 24 | 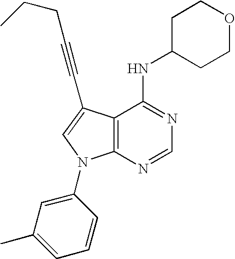 | (5-Pent-1-ynyl-7-m-tolyl-7H- pyrrolo[2,3-d]pyrimidin-4-yl)- (tetrahydro-pyran-4-yl)-amine |
| 25 | 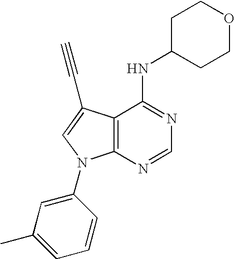 | (5-Ethynyl-7-m-tolyl-7H-pyrrolo[2,3- d]pyrimidin-4-yl)-(tetrahydro-pyran-4- yl)-amine |
| 26 | 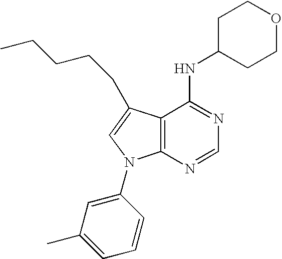 | (5-Pentyl-7-m-tolyl-7H-pyrrolo[2,3- d]pyrimidin-4-yl)-(tetrahydro-pyran-4- yl)-amine |
| 27 | 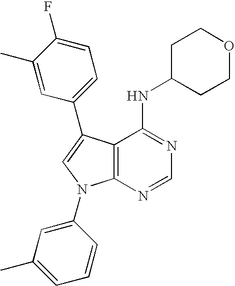 | [5-(4-Fluoro-3-methyl-phenyl)-7-m- tolyl-7H-pyrrolo[2,3-d]pyrimidin-4-yl]- (tetrahydro-pyran-4-yl)-amine |
| 28 | 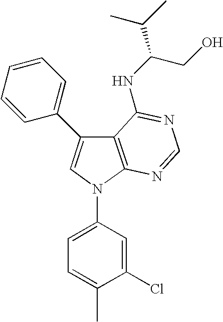 | (R)-2-[7-(3-Chloro-4-methyl-phenyl)- 5-phenyl-7H-pyrrolo[2,3-d]pyrimidin- 4-ylamino]-3-methyl-butan-1-ol |
| 29 | 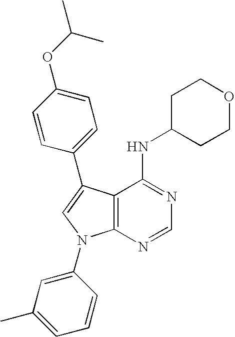 | [5-(4-Isopropoxy-phenyl)-7-m-tolyl- 7H-pyrrolo[2,3-d]pyrimidin-4-yl]- (tetrahydro-pyran-4-yl)-amine |
| 30 | 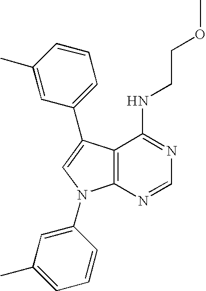 | (5,7-Di-m-tolyl-7H-pyrrolo[2,3- d]pyrimidin-4-yl)-(2-methoxy-ethyl)- amine |
| 31 | 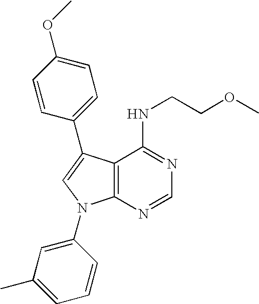 | (2-Methoxy-ethyl)-[5-(4-methoxy- phenyl)-7-m-tolyl-7H-pyrrolo[2,3- d]pyrimidin-4-yl]-amine |
| 32 | 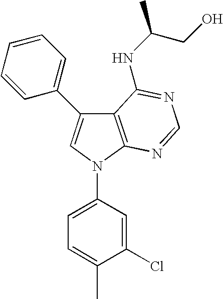 | (S)-2-[7-(3-Chloro-4-methyl-phenyl)-5- phenyl-7H-pyrrolo[2,3-d]pyrimidin-4- ylamino]-propan-1-ol |
| 33 | 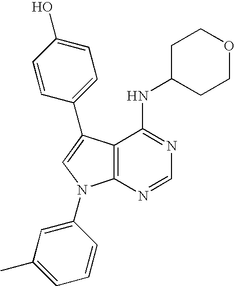 | 4-[4-(Tetrahydro-pyran-4-ylamino)-7- m-tolyl-7H-pyrrolo[2,3-d]pyrimidin-5- yl]-phenol |
| 34 | 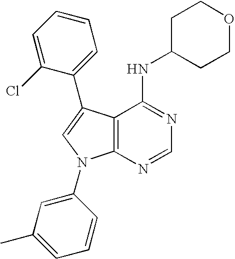 | [5-(2-Chloro-phenyl)-7-m-tolyl-7H- pyrrolo[2,3-d]pyrimidin-4-yl]- (tetrahydro-pyran-4-yl)-amine |
| 35 | 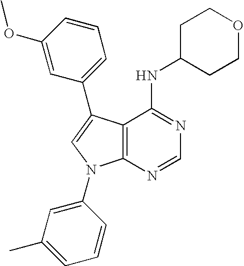 | [5-(3-Methoxy-phenyl)-7-m-tolyl-7H- pyrrolo[2,3-d]pyrimidin-4-yl]- (tetrahydro-pyran-4-yl)-amine |
| 36 | 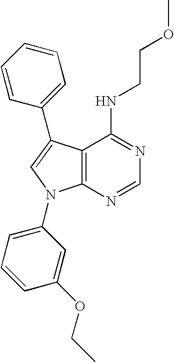 | [7-(3-Ethoxy-phenyl)-5-phenyl-7H- pyrrolo[2,3-d]pyrimidin-4-yl]-(2- methoxy-ethyl)-amine |
| 37 | 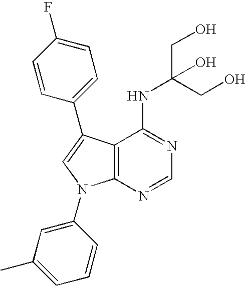 | 2-[5-(4-Fluoro-phenyl)-7-m-tolyl-7H- pyrrolo[2,3-d]pyrimidin-4-ylamino]-2- hydroxymethyl-propane-1,3-diol |
| 38 | 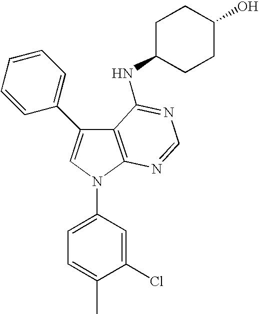 | 4-[7-(3-Chloro-4-methyl-phenyl)-5- phenyl-7H-pyrrolo[2,3-d]pyrimidin-4- ylamino]-cyclohexanol |
| 39 | 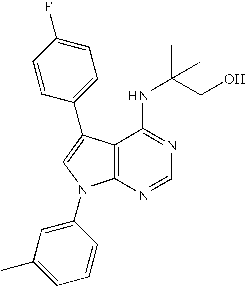 | 2-[5-(4-Fluoro-phenyl)-7-m-tolyl-7H- pyrrolo[2,3-d]pyrimidin-4-ylamino]-2- methyl-propan-1-ol |
| 40 | 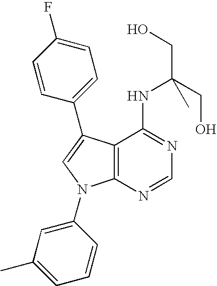 | 2-[5-(4-Fluoro-phenyl)-7-m-tolyl-7H- pyrrolo[2,3-d]pyrimidin-4-ylamino]-2- methyl-propane-1,3-diol; compound with trifluoro-acetic acid |
| 41 | 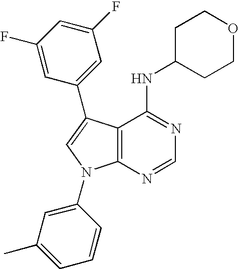 | [5-(3,5-Difluoro-phenyl)-7-m-tolyl-7H- pyrrolo[2,3-d]pyrimidin-4-yl]- (tetrahydro-pyran-4-yl)-amine |
| 42 | 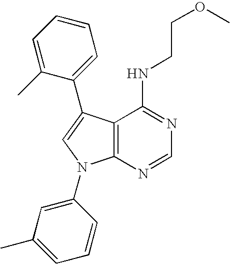 | (2-Methoxy-ethyl)-(7-m-tolyl-5-o- tolyl-7H-pyrrolo[2,3-d]pyrimidin-4-yl)- amine |
| 43 | 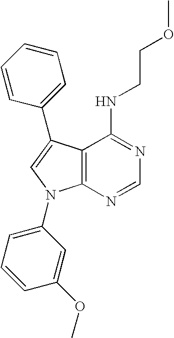 | (2-Methoxy-ethyl)-[7-(3-methoxy- phenyl)-5-phenyl-7H-pyrrolo[2,3- d]pyrimidin-4-yl]-amine |
| 44 | 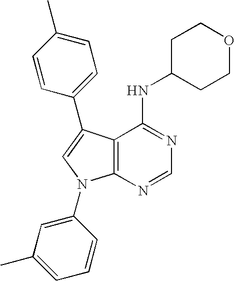 | (Tetrahydro-pyran-4-yl)-(7-m-tolyl-5- p-tolyl-7H-pyrrolo[2,3-d]pyrimidin-4- yl)-amine |
| 45 | 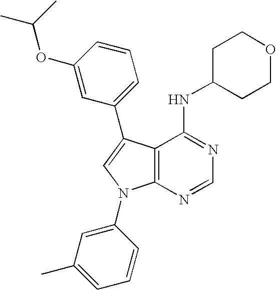 | [5-(3-Isopropoxy-phenyl)-7-m-tolyl- 7H-pyrrolo[2,3-d]pyrimidin-4-yl]- (tetrahydro-pyran-4-yl)-amine |
| 46 | 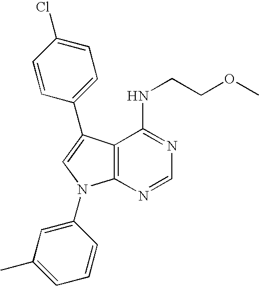 | [5-(4-Chloro-phenyl)-7-m-tolyl-7H- pyrrolo[2,3-d]pyrimidin-4-yl]-(2- methoxy-ethyl)-amine |
| 47 | 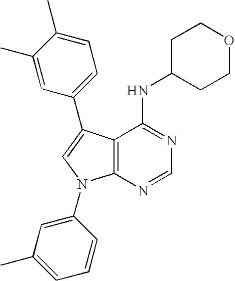 | [5-(3,4-Dimethyl-phenyl)-7-m-tolyl- 7H-pyrrolo[2,3-d]pyrimidin-4-yl]- (tetrahydro-pyran-4-yl)-amine |
| 48 | 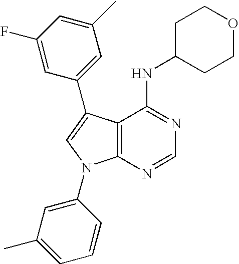 | [5-(3-Fluoro-5-methyl-phenyl)-7-m- tolyl-7H-pyrrolo[2,3-d]pyrimidin-4-yl]- (tetrahydro-pyran-4-yl)-amine |
| 49 | 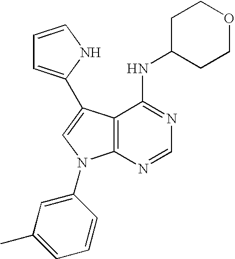 | [5-(1H-Pyrrol-2-yl)-7-m-tolyl-7H- pyrrolo[2,3-d]pyrimidin-4-yl]- (tetrahydro-pyran-4-yl)-amine |
| 50 | 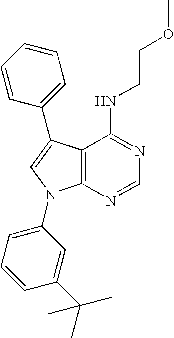 | [7-(3-tert-Butyl-phenyl)-5-phenyl-7H- pyrrolo[2,3-d]pyrimidin-4-yl]-(2- methoxy-ethyl)-amine |
| 51 | 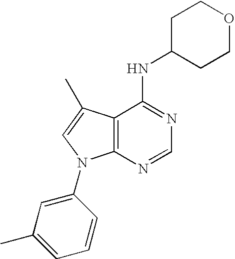 | (5-Methyl-7-m-tolyl-7H-pyrrolo[2,3- d]pyrimidin-4-yl)-(tetrahydro-pyran-4- yl)-amine |
| 52 | 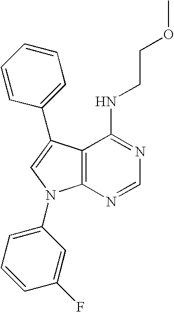 | [7-(3-Fluoro-phenyl)-5-phenyl-7H- pyrrolo[2,3-d]pyrimidin-4-yl]-(2- methoxy-ethyl)-amine |
| 53 | 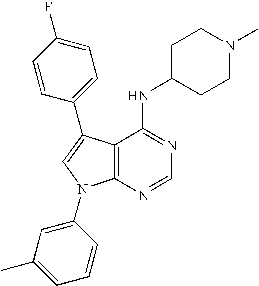 | [5-(4-Fluoro-phenyl)-7-m-tolyl-7H- pyrrolo[2,3-d]pyrimidin-4-yl]-(1- methyl-piperidin-4-yl)-amine |
| 54 | 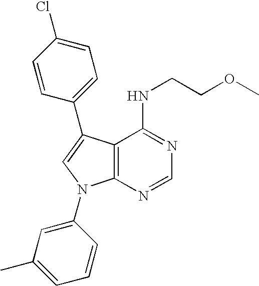 | [5-(4-Chloro-phenyl)-7-m-tolyl-7H- pyrrolo[2,3-d]pyrimidin-4-yl]-(2- methoxy-ethyl)-amine |
| 55 | 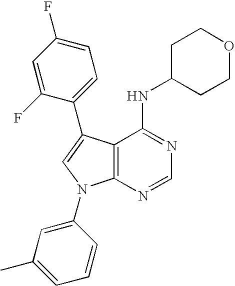 | [5-(2,4-Difluoro-phenyl)-7-m-tolyl-7H- pyrrolo[2,3-d]pyrimidin-4-yl]- (tetrahydro-pyran-4-yl)-amine |
| 56 | 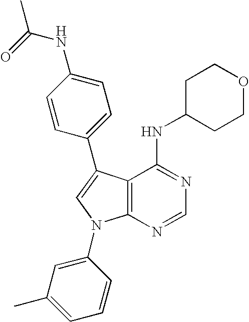 | N-{4-[4-(Tetrahydro-pyran-4-ylamino)- 7-m-tolyl-7H-pyrrolo[2,3-d]pyrimidin- 5-yl]-phenyl}-acetamide |
| 57 | 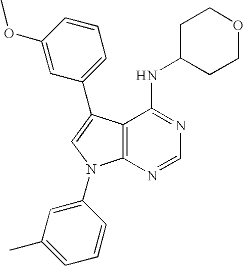 | [5-(3-Methoxy-phenyl)-7-m-tolyl-7H- pyrrolo[2,3-d]pyrimidin-4-yl]- (tetrahydro-pyran-4-yl)-amine |
| 58 | 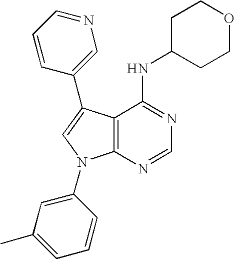 | (5-Pyridin-3-yl-7-m-tolyl-7H- pyrrolo[2,3-d]pyrimidin-4-yl)- (tetrahydro-pyran-4-yl)-amine |
| 59 | 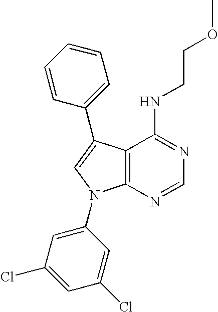 | [7-(3,5-Dichloro-phenyl)-5-phenyl-7H- pyrrolo[2,3-d]pyrimidin-4-yl]-(2- methoxy-ethyl)-amine |
| 60 | 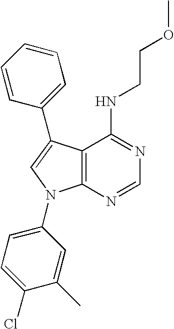 | [7-(4-Chloro-3-methyl-phenyl)-5- phenyl-7H-pyrrolo[2,3-d]pyrimidin-4- yl]-(2-methoxy-ethyl)-amine |
| 61 | 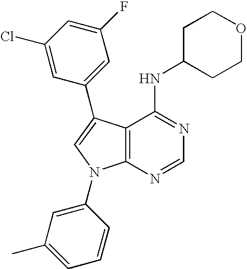 | [5-(3-Chloro-5-fluoro-phenyl)-7-m- tolyl-7H-pyrrolo[2,3-d]pyrimidin-4-yl]- (tetrahydro-pyran-4-yl)-amine |
| 62 | 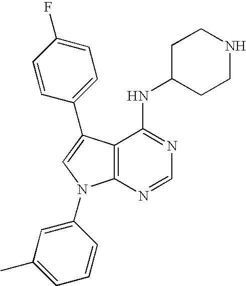 | [5-(4-Fluoro-phenyl)-7-m-tolyl-7H- pyrrolo[2,3-d]pyrimidin-4-yl]- piperidin-4-yl-amine |
| 63 | 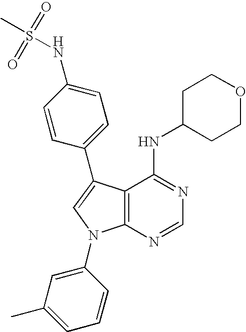 | N-{4-[4-(Tetrahydro-pyran-4-ylamino)- 7-m-tolyl-7H-pyrrolo[2,3-d]pyrimidin- 5-yl]-phenyl}-methanesulfonamide |
| 64 | 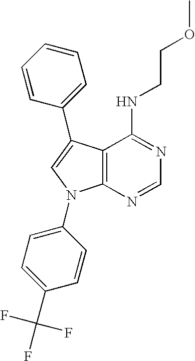 | (2-Methoxy-ethyl)-[5-phenyl-7-(4- trifluoromethyl-phenyl)-7H- pyrrolo[2,3-d]pyrimidin-4-yl]-amine |
| 65 | 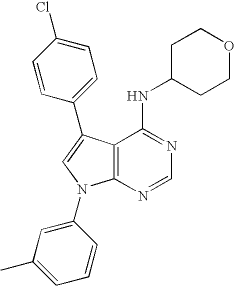 | [5-(4-Chloro-phenyl)-7-m-tolyl-7H- pyrrolo[2,3-d]pyrimidin-4-yl]- (tetrahydro-pyran-4-yl)-amine |
| 66 | 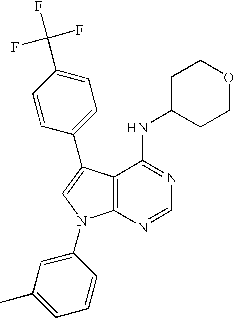 | (Tetrahydro-pyran-4-yl)-[7-m-tolyl-5- (4-trifluoromethyl-phenyl)-7H- pyrrolo[2,3-d]pyrimidin-4-yl]-amine |
| 67 | 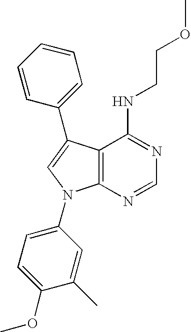 | (2-Methoxy-ethyl)-[7-(4-methoxy-3- methyl-phenyl)-5-phenyl-7H- pyrrolo[2,3-d]pyrimidin-4-yl]-amine |
| 68 | 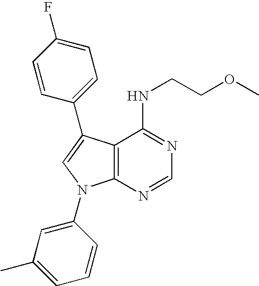 | [5-(4-Fluoro-phenyl)-7-m-tolyl-7H- pyrrolo[2,3-d]pyrimidin-4-yl]-(2- methoxy-ethyl)-amine |
| 69 | 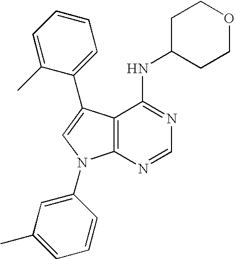 | (Tetrahydro-pyran-4-yl)-(7-m-tolyl-5-o- tolyl-7H-pyrrolo[2,3-d]pyrimidin-4-yl)- amine |
| 70 | 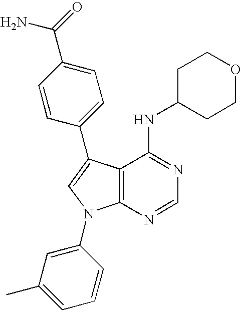 | 4-[4-(Tetrahydro-pyran-4-ylamino)-7- m-tolyl-7H-pyrrolo[2,3-d]pyrimidin-5- yl]-benzamide |
| 71 | 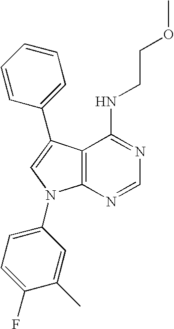 | [7-(4-Fluoro-3-methyl-phenyl)-5- phenyl-7H-pyrrolo[2,3-d]pyrimidin-4- yl]-(2-methoxy-ethyl)-amine |
| 72 | 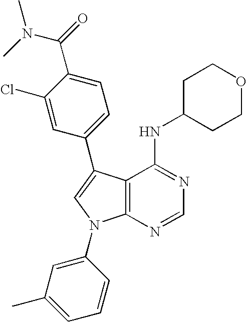 | 2-Chloro-N,N-dimethyl-4-[4- (tetrahydro-pyran-4-ylamino)-7-m- tolyl-7H-pyrrolo[2,3-d]pyrimidin-5-yl]- benzamide |
| 73 | 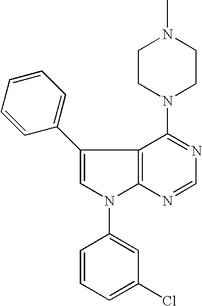 | 7-(3-Chloro-phenyl)-4-(4-methyl- piperazin-1-yl)-5-phenyl-7H- pyrrolo[2,3-d]pyrimidine |
| 74 | 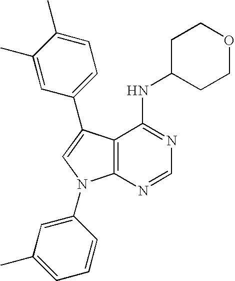 | [5-(3,4-Dimethyl-phenyl)-7-m-tolyl- 7H-pyrrolo[2,3-d]pyrimidin-4-yl]- (tetrahydro-pyran-4-yl)-amine |
| 75 | 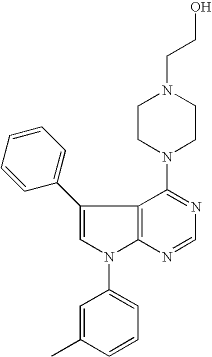 | 2-[4-(5-Phenyl-7-m-tolyl-7H- pyrrolo[2,3-d]pyrimidin-4-yl)- piperazin-1-yl]-ethanol |
| 76 | 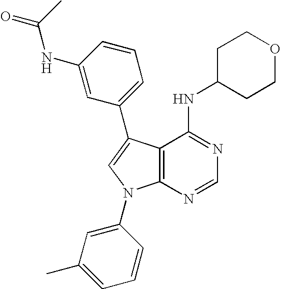 | N-{3-[4-(Tetrahydro-pyran-4-ylamino)- 7-m-tolyl-7H-pyrrolo[2,3-d]pyrimidin- 5-yl]-phenyl}-acetamide |
| 77 | 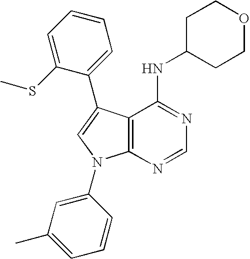 | [5-(2-Methylsulfanyl-phenyl)-7-m- tolyl-7H-pyrrolo[2,3-d]pyrimidin-4-yl]- (tetrahydro-pyran-4-yl)-amine |
| 78 | 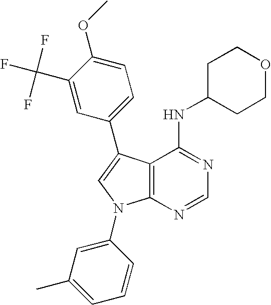 | [5-(4-Methoxy-3-trifluoromethyl- phenyl)-7-m-tolyl-7H-pyrrolo[2,3- d]pyrimidin-4-yl]-(tetrahydro-pyran-4- yl)-amine |
| 79 | 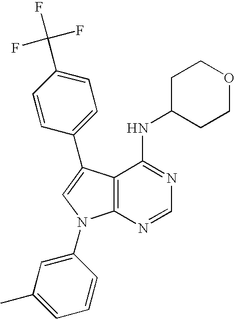 | (Tetrahydro-pyran-4-yl)-[7-m-tolyl-5- (4-trifluoromethyl-phenyl)-7H- pyrrolo[2,3-d]pyrimidin-4-yl]-amine |
| 80 | 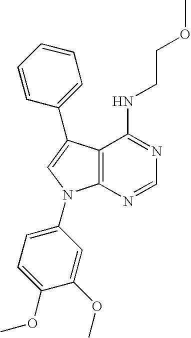 | [7-(3,4-Dimethoxy-phenyl)-5-phenyl- 7H-pyrrolo[2,3-d]pyrimidin-4-yl]-(2- methoxy-ethyl)-amine |
| 81 | 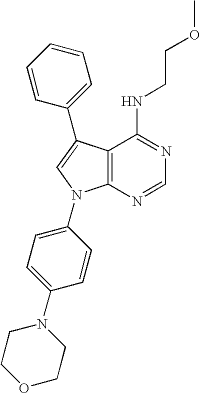 | (2-Methoxy-ethyl)-[7-(4-morpholin-4- yl-phenyl)-5-phenyl-7H-pyrrolo[2,3- d]pyrimidin-4-yl]-amine |
| 82 | 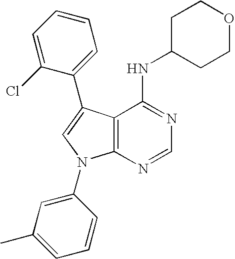 | [5-(2-Chloro-phenyl)-7-m-tolyl-7H- pyrrolo[2,3-d]pyrimidin-4-yl]- (tetrahydro-pyran-4-yl)-amine |
| 83 | 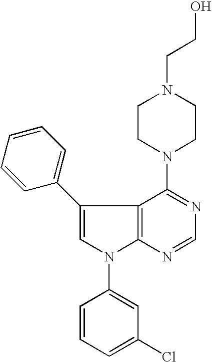 | 2-{4-[7-(3-Chloro-phenyl)-5-phenyl- 7H-pyrrolo[2,3-d]pyrimidin-4-yl]- piperazin-1-yl}-ethanol |
| 84 | 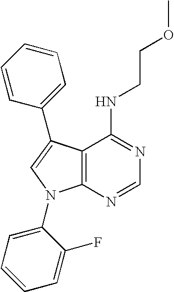 | [7-(2-Fluoro-phenyl)-5-phenyl-7H- pyrrolo[2,3-d]pyrimidin-4-yl]-(2- methoxy-ethyl)-amine |
| 85 | 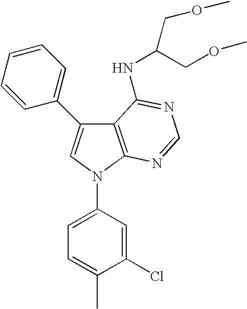 | [7-(3-Chloro-4-methyl-phenyl)-5- phenyl-7H-pyrrolo[2,3-d]pyrimidin-4- yl]-(2-methoxy-1-methoxymethyl- ethyl)-amine |
| 86 | 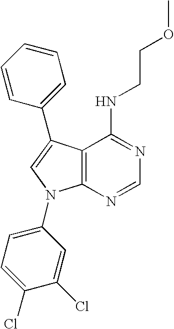 | [7-(3,4-Dichloro-phenyl)-5-phenyl-7H- pyrrolo[2,3-d]pyrimidin-4-yl]-(2- methoxy-ethyl)-amine |
| 87 | 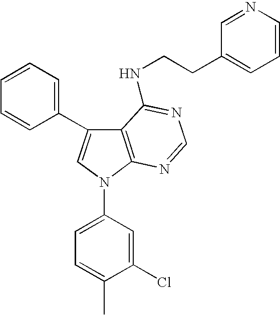 | [7-(3-Chloro-4-methyl-phenyl)-5- phenyl-7H-pyrrolo[2,3-d]pyrimidin-4- yl]-(2-pyridin-3-yl-ethyl)-amine |
| 88 | 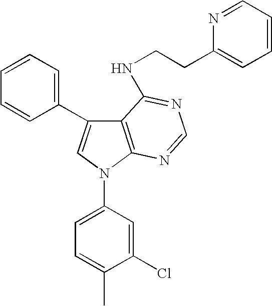 | [7-(3-Chloro-4-methyl-phenyl)-5- phenyl-7H-pyrrolo[2,3-d]pyrimidin-4- yl]-(2-pyridin-2-yl-ethyl)-amine |
| 89 | 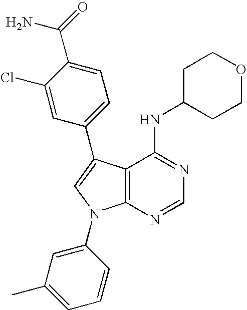 | 2-Chloro-4-[4-(tetrahydro-pyran-4- ylamino)-7-m-tolyl-7H-pyrrolo[2,3- d]pyrimidin-5-yl]-benzamide |
| 90 | 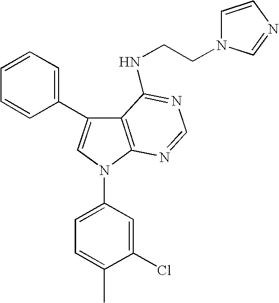 | [7-(3-Chloro-4-methyl-phenyl)-5- phenyl-7H-pyrrolo[2,3-d]pyrimidin-4- yl]-(2-imidazol-1-yl-ethyl)-amine |
| 91 | 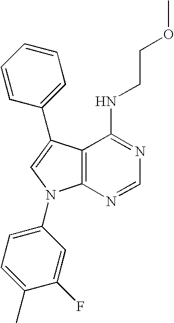 | [7-(3-Fluoro-4-methyl-phenyl)-5- phenyl-7H-pyrrolo[2,3-d]pyrimidin-4- yl]-(2-methoxy-ethyl)-amine |
| 92 | 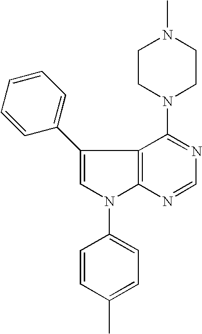 | 4-(4-Methyl-piperazin-1-yl)-5-phenyl- 7-p-tolyl-7H-pyrrolo[2,3-d]pyrimidine |
| 93 | 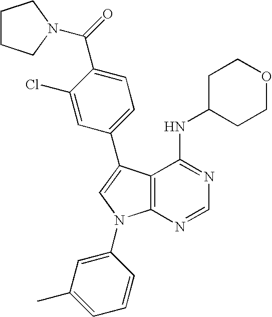 | {2-Chloro-4-[4-(tetrahydro-pyran-4- ylamino)-7-m-tolyl-7H-pyrrolo[2,3- d]pyrimidin-5-yl]-phenyl}-pyrrolidin- 1-yl-methanone |
| 94 | 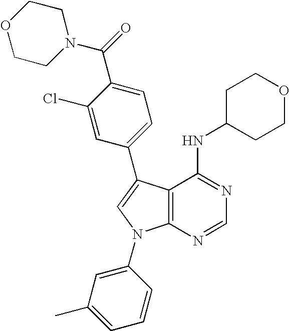 | {2-Chloro-4-[4-(tetrahydro-pyran-4- ylamino)-7-m-tolyl-7H-pyrrolo[2,3- d]pyrimidin-5-yl]-phenyl}-morpholin- 4-yl-methanone |
| 95 | 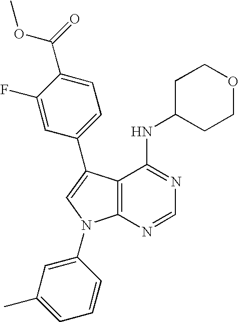 | 2-Fluoro-4-[4-(tetrahydro-pyran-4- ylamino)-7-m-tolyl-7H-pyrrolo[2,3- d]pyrimidin-5-yl]-benzoic acid methyl ester |
| 96 | 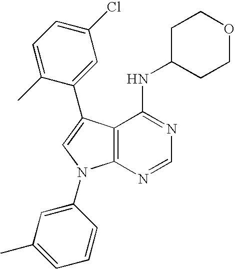 | [5-(5-Chloro-2-methyl-phenyl)-7-m- tolyl-7H-pyrrolo[2,3-d]pyrimidin-4-yl]- (tetrahydro-pyran-4-yl)-amine |
| 97 | 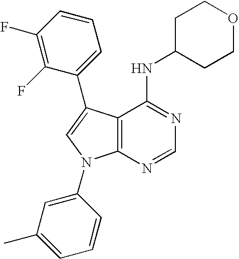 | [5-(2,3-Difluoro-phenyl)-7-m-tolyl- 7H-pyrrolo[2,3-d]pyrimidin-4-yl]- (tetrahydro-pyran-4-yl)-amine |
| 98 | 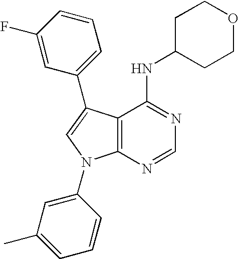 | [5-(3-Fluoro-phenyl)-7-m-tolyl-7H- pyrrolo[2,3-d]pyrimidin-4-yl]- (tetrahydro-pyran-4-yl)-amine |
| 99 | 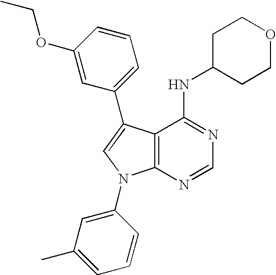 | [5-(3-Ethoxy-phenyl)-7-m-tolyl-7H- pyrrolo[2,3-d]pyrimidin-4-yl]- (tetrahydro-pyran-4-yl)-amine |
| 100 | 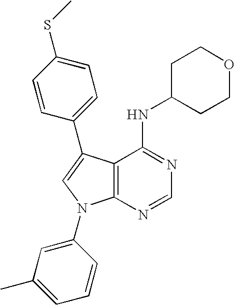 | [5-(4-Methylsulfanyl-phenyl)-7-m- tolyl-7H-pyrrolo[2,3-d]pyrimidin-4-yl]- (tetrahydro-pyran-4-yl)-amine |
| 101 | 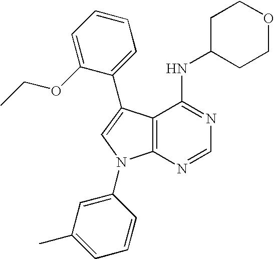 | [5-(2-Ethoxy-phenyl)-7-m-tolyl-7H- pyrrolo[2,3-d]pyrimidin-4-yl]- (tetrahydro-pyran-4-yl)-amine |
| 102 | 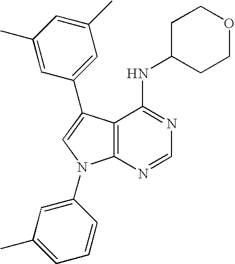 | [5-(3,5-Dimethyl-phenyl)-7-m-tolyl- 7H-pyrrolo[2,3-d]pyrimidin-4-yl]- (tetrahydro-pyran-4-yl)-amine |
| 103 | 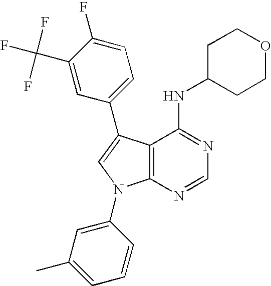 | [5-(4-Fluoro-3-trifluoromethyl- phenyl)-7-m-tolyl-7H-pyrrolo[2,3- d]pyrimidin-4-yl]-(tetrahydro-pyran-4- yl)-amine |
| 104 | 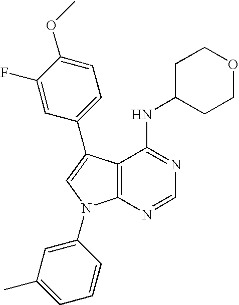 | [5-(3-Fluoro-4-methoxy-phenyl)-7-m- tolyl-7H-pyrrolo[2,3-d]pyrimidin-4-yl]- (tetrahydro-pyran-4-yl)-amine |
| 105 | 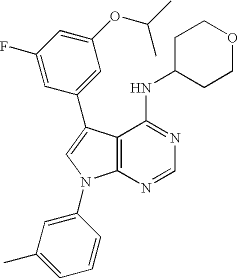 | [5-(3-Fluoro-5-isopropoxy-phenyl)-7- m-tolyl-7H-pyrrolo[2,3-d]pyrimidin-5- yl]-(tetrahydro-pyran-4-yl)-amine |
| 106 | 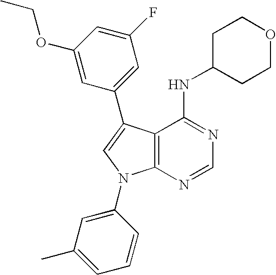 | [5-(3-Ethoxy-5-fluoro-phenyl)-7-m- tolyl-7H-pyrrolo[2,3-d]pyrimidin-4-yl]- (tetrahydro-pyran-4-yl)-amine |
| 107 | 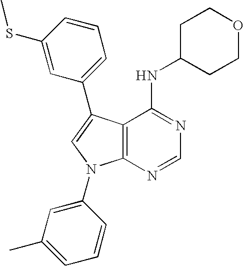 | [5-(3-Methylsulfanyl-phenyl)-7-m- tolyl-7H-pyrrolo[2,3-d]pyrimidin-4-yl]- (tetrahydro-pyran-4-yl)-amine |
| 108 | 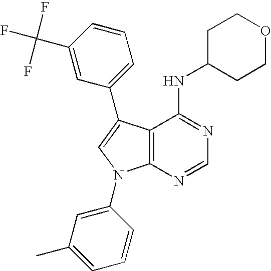 | (Tetrahydro-pyran-4-yl)-[7-m-tolyl-5- (3-trifluoromethyl-phenyl)-7H- pyrrolo[2,3-d]pyrimidin-4-yl]-amine |
| 109 | 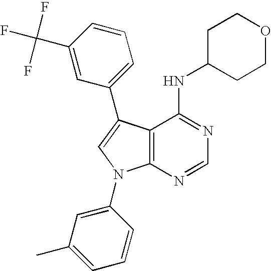 | (Tetrahydro-pyran-4-yl)-[7-m-tolyl-5- (3-trifluoromethyl-phenyl)-7H- pyrrolo[2,3-d]pyrimidin-4-yl]-amine |
| 110 | 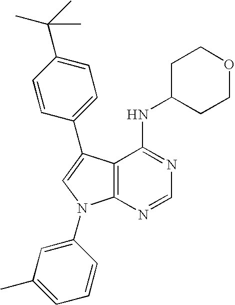 | [5-(4-tert-Butyl-phenyl)-7-m-tolyl-7H- pyrrolo[2,3-d]pyrimidin-4-yl]- (tetrahydro-pyran-4-yl)-amine |
| 111 | 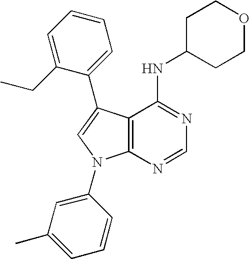 | [5-(2-Ethyl-phenyl)-7-m-tolyl-7H- pyrrolo[2,3-d]pyrimidin-4-yl]- (tetrahydro-pyran-4-yl)-amine |
| 112 | 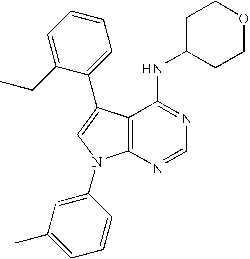 | [5-(2-Ethyl-phenyl)-7-m-tolyl-7H- pyrrolo[2,3-d]pyrimidin-4-yl]- (tetrahydro-pyran-4-yl)-amine |
| 113 | 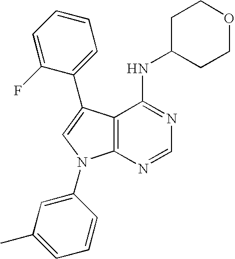 | [5-(2-Fluoro-phenyl)-7-m-tolyl-7H- pyrrolo[2,3-d]pyrimidin-4-yl]- (tetrahydro-pyran-4-yl)-amine |
| 114 | 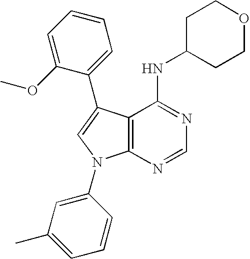 | [5-(2-Methoxy-phenyl)-7-m-tolyl-7H- pyrrolo[2,3-d]pyrimidin-4-yl]- (tetrahydro-pyran-4-yl)-amine |
| 115 | 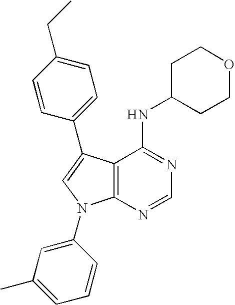 | [5-(4-Ethyl-phenyl)-7-m-tolyl-7H- pyrrolo[2,3-d]pyrimidin-4-yl]- (tetrahydro-pyran-4-yl)-amine |
| 116 | 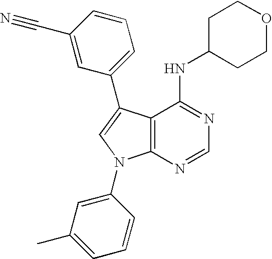 | 3-[4-(Tetrahydro-pyran-4-ylamino)-7- m-tolyl-7H-pyrrolo[2,3-d]pyrimidin-5- yl]-benzonitrile |
| 117 | 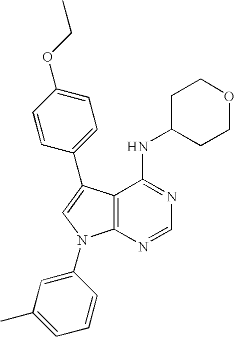 | [5-(4-Ethoxy-phenyl)-7-m-tolyl-7H- pyrrolo[2,3-d]pyrimidin-4-yl]- (tetrahydro-pyran-4-yl)-amine |
| 118 | 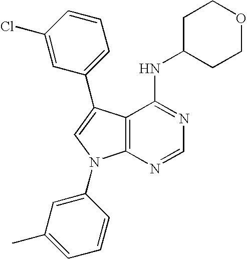 | [5-(3-Chloro-phenyl)-7-m-tolyl-7H- pyrrolo[2,3-d]pyrimidin-4-yl]- (tetrahydro-pyran-4-yl)-amine |
| 119 | 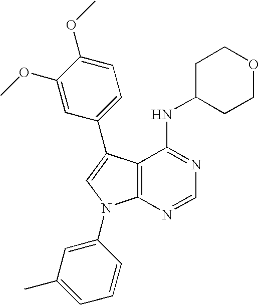 | [5-(3,4-Dimethoxy-phenyl)-7-m-tolyl- 7H-pyrrolo[2,3-d]pyrimidin-4-yl]- (tetrahydro-pyran-4-yl)-amine |
| 120 | 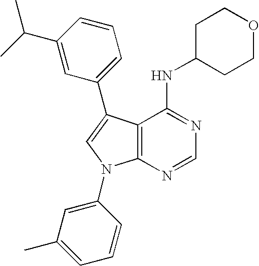 | [5-(3-Isopropyl-phenyl)-7-m-tolyl-7H- pyrrolo[2,3-d]pyrimidin-4-yl]- (tetrahydro-pyran-4-yl)-amine |
| 121 | 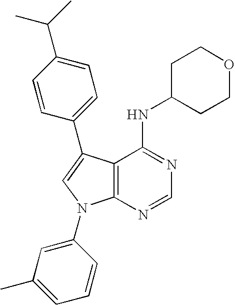 | [5-(4-Isopropyl-phenyl)-7-m-tolyl-7H- pyrrolo[2,3-d]pyrimidin-4-yl]- (tetrahydro-pyran-4-yl)-amine |
| 122 | 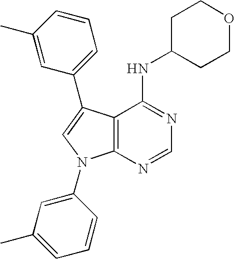 | (5,7-Di-m-tolyl-7H-pyrrolo[2,3- d]pyrimidin-4-yl)-(tetrahydro-pyran-4- yl)-amine |
| 123 | 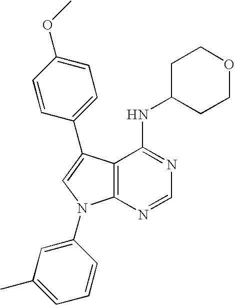 | [5-(4-Methoxy-phenyl)-7-m-tolyl-7H- pyrrolo[2,3-d]pyrimidin-4-yl]- (tetrahydro-pyran-4-yl)-amine |
| 124 | 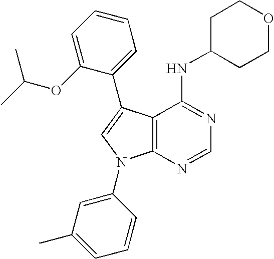 | [5-(2-Isopropoxy-phenyl)-7-m-tolyl- 7H-pyrrolo[2,3-d]pyrimidin-4-yl]- (tetrahydro-pyran-4-yl)-amine |
| 125 | 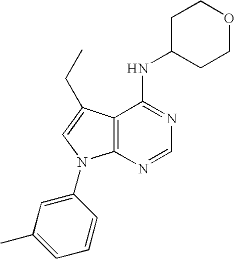 | (5-Ethyl-7-m-tolyl-7H-pyrrolo[2,3- d]pyrimidin-4-yl)-(tetrahydro-pyran-4- yl)-amine |
| 126 | 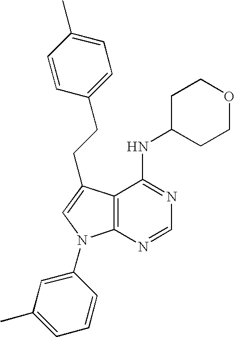 | (Tetrahydro-pyran-4-yl)-[7-m-tolyl-5- (2-p-tolyl-ethyl)-7H-pyrrolo[2,3- d]pyrimidin-4-yl]-amine |
| 127 | 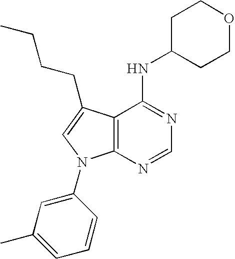 | (5-Butyl-7-m-tolyl-7H-pyrrolo[2,3- d]pyrimidin-4-yl)-(tetrahydro-pyran-4- yl)-amine |
| 128 | 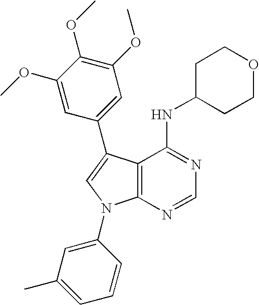 | (Tetrahydro-pyran-4-yl)-[7-m-tolyl-5- (3,4,5-trimethoxy-phenyl)-7H- pyrrolo[2,3-d]pyrimidin-4-yl]-amine |
| 129 | 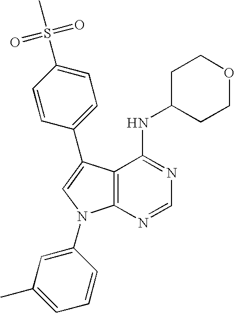 | [5-(4-Methanesulfonyl-phenyl)-7-m- tolyl-7H-pyrrolo[2,3-d]pyrimidin-4-yl]- (tetrahydro-pyran-4-yl)-amine |
| 130 | 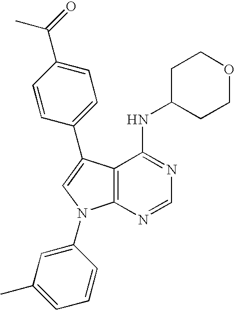 | 1-{4-[4-(Tetrahydro-pyran-4- ylamino)-7-m-tolyl-7H-pyrrolo[2,3- d]pyrimidin-5-yl]-phenyl}-ethanone |
| 131 | 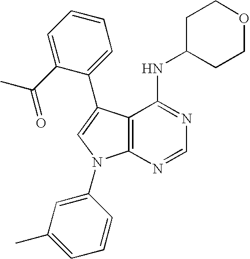 | 1-{2-[4-(Tetrahydro-pyran-4- ylamino)-7-m-tolyl-7H-pyrrolo[2,3- d]pyrimidin-5-yl]-phenyl}-ethanone |
| 132 | 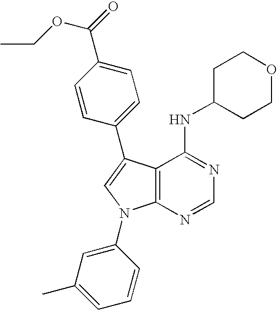 | 4-[4-(Tetrahydro-pyran-4-ylamino)-7- m-tolyl-7H-pyrrolo[2,3-d]pyrimidin-5- yl]-benzoic acid ethyl ester |
| 133 | 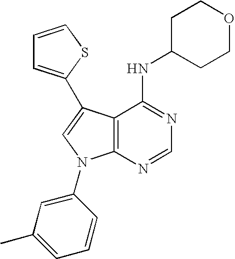 | (Tetrahydro-pyran-4-yl)-(5-thiophen- 2-yl-7-m-tolyl-7H-pyrrolo[2,3- d]pyrimidin-4-yl)-amine |
| 134 | 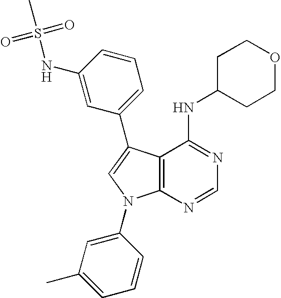 | N-{3-[4-(Tetrahydro-pyran-4- ylamino)-7-m-tolyl-7H-pyrrolo[2,3- d]pyrimidin-5-yl]-phenyl}- methanesulfonamide |
| 135 | 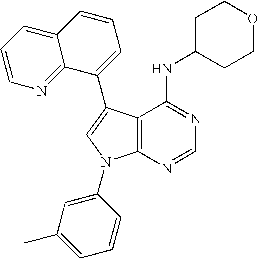 | (5-Quinolin-8-yl-7-m-tolyl-7H- pyrrolo[2,3-d]pyrimidin-4-yl)- (tetrahydro-pyran-4-yl)-amine |
| 136 | 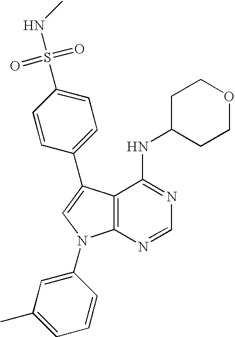 | N-Methyl-4-[4-(tetrahydro-pyran-4- ylamino)-7-m-tolyl-7H-pyrrolo[2,3- d]pyrimidin-5-yl]-benzenesulfonamide |
| 137 | 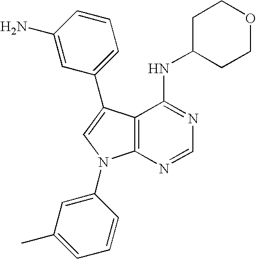 | [5-(3-Amino-phenyl)-7-m-tolyl-7H- pyrrolo[2,3-d]pyrimidin-4-yl]- (tetrahydro-pyran-4-yl)-amine |
| 138 | 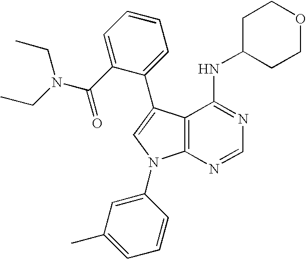 | N,N-Diethyl-2-[4-(tetrahydro-pyran-4- ylamino)-7-m-tolyl-7H-pyrrolo[2,3- d]pyrimidin-5-yl]-benzamide |
| 139 | 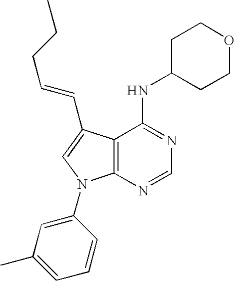 | [5-((E)-Pent-1-enyl)-7-m-tolyl-7H- pyrrolo[2,3-d]pyrimidin-4-yl]- (tetrahydro-pyran-4-yl)-amine |
| 140 | 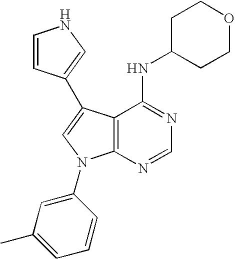 | [5-(1H-Pyrrol-3-y)-7-m-tolyl-7H- pyrrolo[2,3-d]pyrimidin-4-yl]- (tetrahydro-pyran-4-yl)-amine |
| 141 | 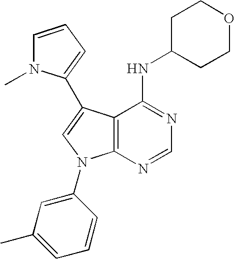 | [5-(1-Methyl-1H-pyrrol-2-yl)-7-m- tolyl-7H-pyrrolo[2,3-d]pyrimidin-4-yl]- (tetrahydro-pyran-4-yl)-amine |
| 142 | 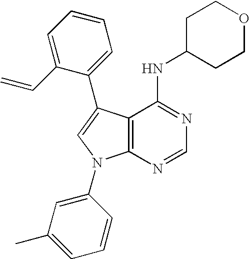 | (Tetrahydro-pyran-4-yl-[7-m-tolyl-5- (2-vinyl-phenyl)-7H-pyrrolo[2,3- d]pyrimidin-4-yl]-amine |
| 143 | 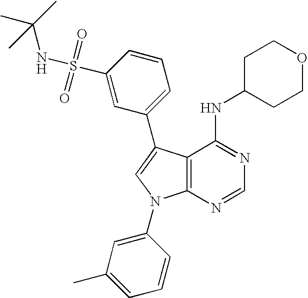 | N-tert-Butyl-3-[4-(tetrahydro-pyran-4- ylamino)-7-m-tolyl-7H-pyrrolo[2,3- d]pyrimidin-5-yl]-benzenesulfonamide |
| 144 | 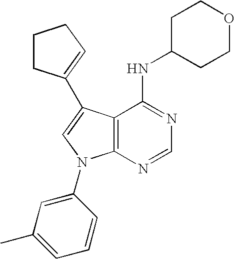 | (5-Cyclopent-1-enyl-7-m-tolyl-7H- pyrrolo[2,3-d]pyrimidin-4-yl)- (tetrahydro-pyran-4-yl)-amine |
| 145 | 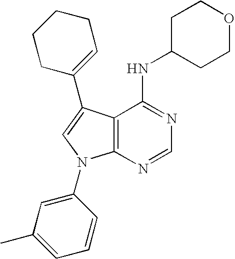 | (5-Cyclohex-1-enyl-7-m-tolyl-7H- pyrrolo[2,3-d]pyrimidin-4-yl)- (tetrahydro-pyran-4-yl)-amine |
| 146 | 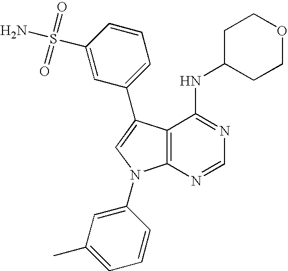 | 3-[4-(Tetrahydro-pyran-4-ylamino)-7- m-tolyl-7H-pyrrolo[2,3-d]pyrimidin-5- yl]-benzenesulfonamide |
| 147 | 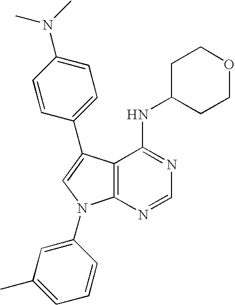 | [5-(4-Dimethylamino-phenyl)-7-m- tolyl-7H-pyrrolo[2,3-d]pyrimidin-4-yl]- (tetrahydro-pyran-4-yl)-amine |
| 148 | 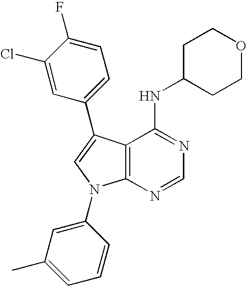 | [5-(3-Chloro-4-fluoro-phenyl)-7-m- tolyl-7H-pyrrolo[2,3-d]pyrimidin-4-yl]- (tetrahydro-pyran-4-yl)-amine |
| 149 | 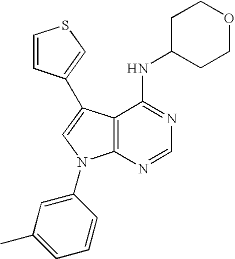 | (Tetrahydro-pyran-4-yl)-(5-thiophen- 3-yl-7-m-tolyl-7H-pyrrolo[2,3- d]pyrimidin-4-yl)-amine |
| 150 | 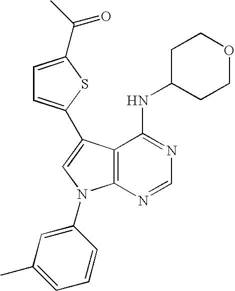 | 1-{5-[4-(Tetrahydro-pyran-4- ylamino)-7-m-tolyl-7H-pyrrolo[2,3- d]pyrimidin-5-yl]-thiophen-2-yl}- ethanone |
| 151 | 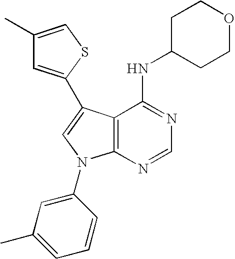 | [5-(4-Methyl-thiophen-2-yl)-7-m-tolyl- 7H-pyrrolo[2,3-d]pyrimidin-4-yl]- (tetrahydro-pyran-4-yl)-amine |
| 152 | 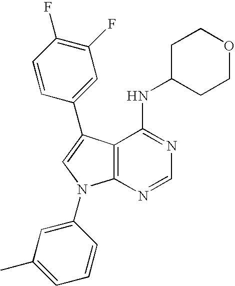 | [5-(3,4-Difluoro-phenyl)-7-m-tolyl- 7H-pyrrolo[2,3-d]pyrimidin-4-yl]- (tetrahydro-pyran-4-yl)-amine |
| 153 | 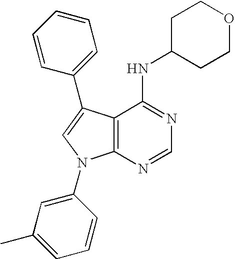 | (5-Phenyl-7-m-tolyl-7H-pyrrolo[2,3- d]pyrimidin-4-yl)-(tetrahydro-pyran-4- yl)-amine |
| 154 | 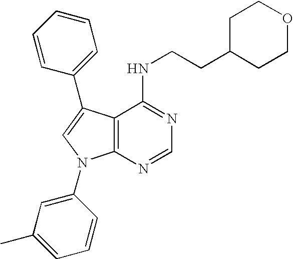 | (5-Phenyl-7-m-tolyl-7H-pyrrolo[2,3- d]pyrimidin-4-yl)-[2-(tetrahydro-pyran- 4-yl)-ethyl]-amine |
| 155 | 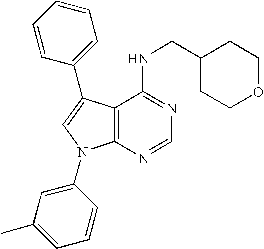 | (5-Phenyl-7-m-tolyl-7H-pyrrolo[2,3- d]pyrimidin-4-yl)-(tetrahydro-pyran-4- ylmethyl)-amine |
| 156 | 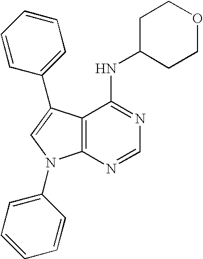 | (5,7-Diphenyl-7H-pyrrolo[2,3- d]pyrimidin-4-yl)-(tetrahydro-pyran-4- yl)-amine |
| 157 | 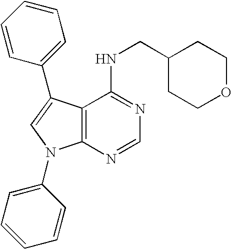 | (5,7-Diphenyl-7H-pyrrolo[2,3- d]pyrimidin-4-yl)-(tetrahydro-pyran-4- ylmethyl)-amine |
| 158 | 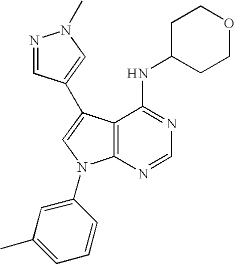 | [5-(1-Methyl-1H-pyrazol-4-yl)-7-m- tolyl-7H-pyrrolo[2,3-d]pyrimidin-4-yl]- (tetrahydro-pyran-4-yl)-amine |
| 159 | 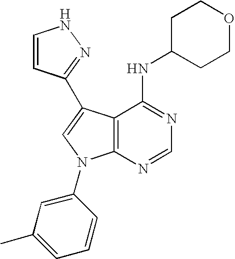 | [5-(1H-Pyran-3-yl)-7-m-tolyl-7H- pyrrolo[2,3-d]pyrimidin-4-yl]- (tetrahydro-pyran-4-yl)-amine |
| 160 | 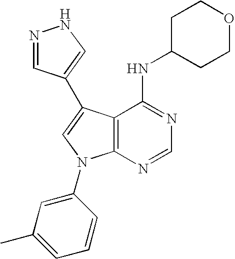 | [5-(1H-Pyrazol-4-yl)-7-m-tolyl-7H- pyrrolo[2,3-d]pyrimidin-4-yl]- (tetrahydro-pyran-4-yl)-amine |
| 161 | 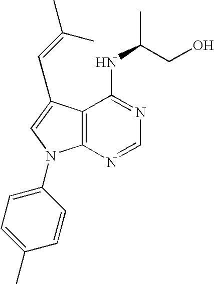 | (S)-2-[5-(2-Methyl-propenyl)-7-p- tolyl-7H-pyrrolo[2,3-d]pyrimidin-4- ylamino]-propan-1-ol |
| 162 | 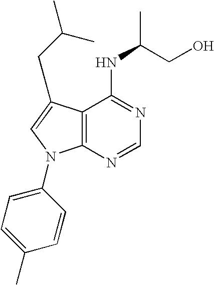 | (S)-2-(5-Isobutyl-7-p-tolyl-7H- pyrrolo[2,3-d]pyrimidin-4-ylamino)- propan-1-ol |

### Synthesis

Compounds of the present invention can be made by a variety of methods depicted in the illustrative synthetic reaction schemes shown and described below.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

### Utility

The compounds of the invention are usable for the treatment of a wide range of genitorurinary diseases, conditions and disorders, including urinary tract disease states associated with bladder outlet obstruction and urinary incontinence conditions such as reduced bladder capacity, frequency of micturition, urge incontinence, stress incontinence, bladder hyperreactivity, benign prostatic hypertrophy (BPH), prostatitis, detrusor hyperreflexia, urinary frequency, nocturia, urinary urgency, overactive bladder, pelvic hypersensitivity, urethritis, prostatitits, pelvic pain syndrome, prostatodynia, cystitis, and idiophatic bladder hypersensitivity, and other symptoms related to overactive bladder.

The compounds of the invention are expected to find utility as analgesics in the treatment of diseases and conditions associated with pain from a wide variety of causes, including, but not limited to, inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injuries (including fractures and sports injuries), and pain associated with functional bowel disorders such as irritable bowel syndrome.

Further, compounds of the invention are useful for treating respiratory disorders, including chronic obstructive pulmonary disorder (COPD), asthma, bronchospasm, and the like.

Additionally, compounds of the invention are useful for treating gastrointestinal disorders, including Irritable Bowel Syndrome (IBS), Inflammatory Bowel Disease (IBD), biliary colic and other biliary disorders, renal colic, diarrhea-dominant IBS, pain associated with GI distension, and the like.

### Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention will be administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

The subject compounds may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described below.

### Examples

The following preparations and examples are given to enable those skilled in the art to more clearly understand and to practice the present invention. They should not be considered as limiting the scope of the invention, but merely as being illustrative and representative thereof.

Unless otherwise stated, all temperatures including melting points (i.e., MP) are in degrees celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product. The following abbreviations may be used in the Preparations and Examples.

### Abbreviations

DBU 1,8-diazabicyclo[5.4.0]undec-7-ene
DCM dichloromethane/methylene chloride
DMF N,N-dimethylformamide
DMAP 4-dimethylaminopyridine
EDCI 1-ethyl-3-(3′-dimethylaminopropyl)carbodiimide
EtOAc ethyl acetate
EtOH ethanol
gc gas chromatography
HMPA hexamethylphosphoramide
HOBt N-Hydroxybenzotriazole
hplc high performance liquid chromatography
mCPBA m-chloroperbenzoic acid
MeCN acetonitrile
NMM N-methyl-morpholine
NMP N-methyl pyrrolidinone
TEA triethylamine
THF tetrahydrofuran
LDA lithium diisopropylamine
TLC thin layer chromatography

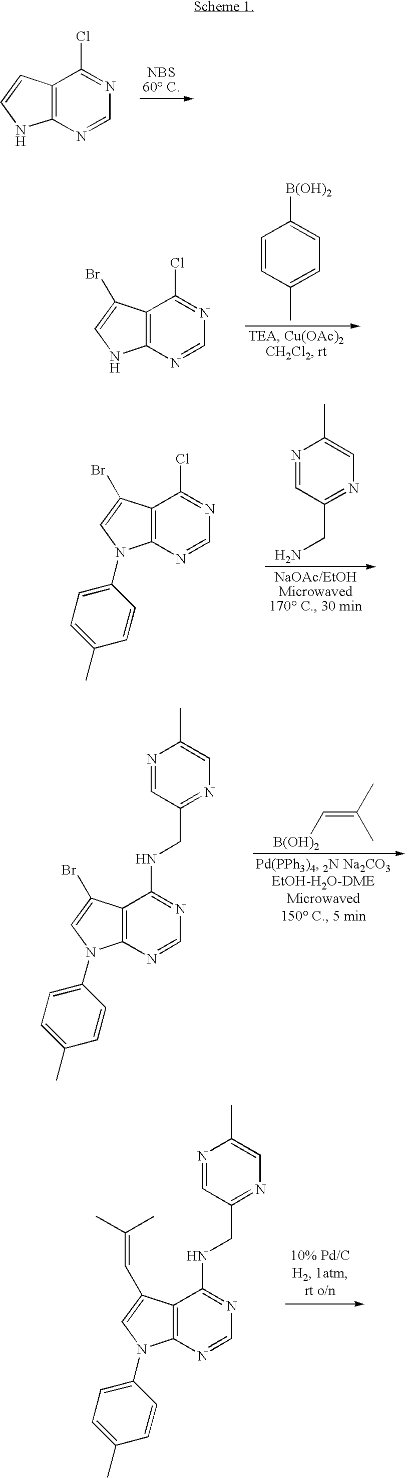

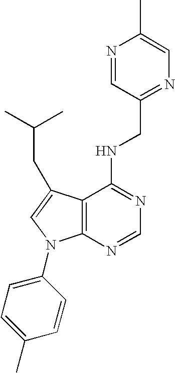

### Example 1

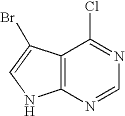

### Preparation of 5-Bromo-4-chloro-7H-pyrrolo[2,3-d]pyrimidine

The solution of 4-chloro-7H-pyrrolo[2,3-d]pyrimidine (6.08 g, 39.07 mmol) in CH₂Cl₂ (100 mL) was heated to 60° C. for for 30 min. A solution of NBS (5.40 g, 39.07 mmol) in CH₂Cl₂ (100 mL) was added slowly in portions to this mixture. The mixture was refluxed for an additional 50 min and allow to cool to rt. Filter off the off white solid and washed with water, dried in vacuum oven to give 7.7 g of 5-bromo-4-chloro-7H-pyrrolo[2,3-d]pyrimidine. MS (ESI) 234.5 (M+H)⁺.

### Example 2

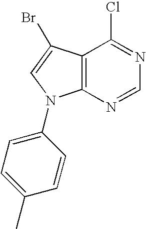

### Preparation of 5-Bromo-4-chloro-7-p-tolyl-7H-pyrrolo[2,3-d]pyrimidine

A round bottom flask was charged with 5-bromo-4-chloro-7H-pyrrolo[2,3-d]pyrimidine (1.66 g, 7.16 mmol), 4-methylbenzeneboronic acid (1.95 g, 14.34 mmol), triethyl amine (2.0 mL, 14.34 mmol), and copper acetate (1.30 g, 7.17 mmol). The vial was flushed with argon, 30 mL of CH₂Cl₂ was added, and the reaction mixture was stirred at rt for o/n. After filtration, the solvent was removed and the brown mass obtained was purified by silica gel chromatography to afford 2.1 g of 5-bromo-4-chloro-7-p-tolyl-7H-pyrrolo[2,3-d]pyrimidine. MS (ESI) 324.3 (M+H)⁺

### Example 3

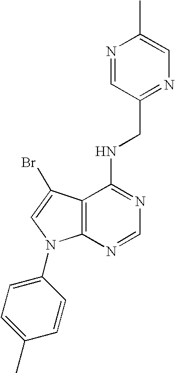

### Preparation of (5-Bromo-7-p-tolyl-7H-pyrrolo[2,3-d]pyrimidin-4-yl)-(5-methyl-pyrazin-2-ylmethyl)-amine

The mixture of 5-bromo-4-chloro-7-p-tolyl-7H-pyrrolo[2,3-d]pyrimidine (0.5 g, 1.55 mmol), C-(5-Methyl-pyrazin-2-yl)-methylamine (0.21 mL, 1.71 mmol), sodium acetate (0.63 g, 4.65 mmol) in ethanol (20 mL) was heated to 170° C. for 30 min in microwave reactor. After filtration, the solvent was removed and the residue obtained was purified by silica gel chromatography to afford 0.55 g of (5-bromo-7-p-tolyl-7H-pyrrolo[2,3-d]pyrimidin-4-yl)-(5-methyl-pyrazin-2-ylmethyl)-amine. MS (ESI) 411.3 (M+H)⁺

### Example 4

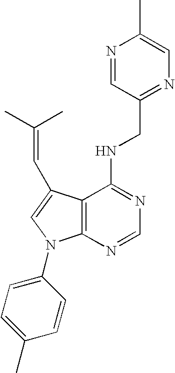

### Preparation of [5-(2-Methyl-propenyl)-7-p-tolyl-7H-pyrrolo[2,3-d]pyrimidin-4-yl]-(4-methyl-pyrazin-2-ylmethyl)-amine

The mixture of (5-bromo-7-isopropyl-7H-pyrrolo[2,3-d]pyrimidin-4-yl)-(5-methyl-pyrazin-2-ylmethyl)-amine (0.36 g, 1.00 mmol), 2,2-dimethylethenylboronic acid (0.13 g, 1.2 mmol), Pd(PPh₃)₄ (60 mg g, 0.05 mmol), Na₂CO₃ (2N, 2 mL, 4 mmol) in ethanol-H₂O-DME (3 mL-4 mL-12 mL) was heated to 150° C. for 5 min in microwave reactor. After filtration, the solvent was removed and the residue obtained was purified by silica gel chromatography to afford 0.29 g of [5-(2-methyl-propenyl)-7-p-tolyl-7H-pyrrolo[2,3-d]pyrimidin-4-yl]-(5-methyl-pyrazin-2-ylmethyl)-amine. MS (ESI) 385.6 (M+H)⁺

### Example 5

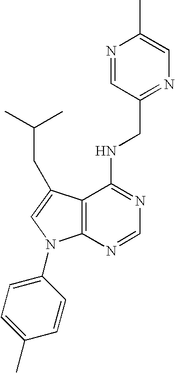

### Preparation of (5-Isobutyl-7-p-tolyl-7H-pyrrolo[2,3-d]pyrimidin-4-yl)-(5-methyl-pyrazin-2-ylmethyl)-amine

The mixture of [5-(2-methyl-propenyl)-7-p-tolyl-7H-pyrrolo[2,3-d]pyrimidin-4-yl]-(5-methyl-pyrazin-2-ylmethyl)-amine (0.30 g, 0.8 mmol), 10% Pd/C (0.1 g) in EtOAc was hydrogenated at 1 atm and rt for o/n. After filtration through a pre-pad celite, the solvent was removed to afford 0.27 g of (5-isobutyl-7-p-tolyl-7H-pyrrolo[2,3-d]pyrimidin-4-yl)-(5-methyl-pyrazin-2-ylmethyl)-amine. MS (ESI) 387.5 (M+H)⁺

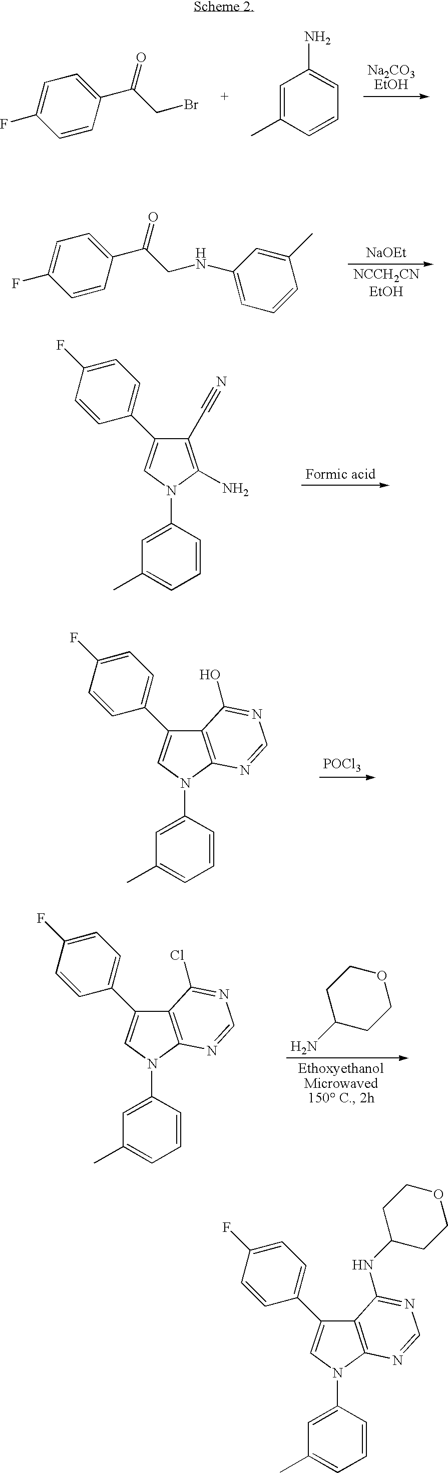

### Example 6

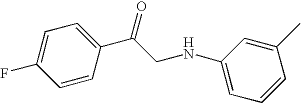

### Preparation of 1-(4-Fluoro-phenyl)-2-m-tolylamino-ethanone

A solution of 2-bromo-1-(4-fluoro-phenyl)-ethanone (14.0 g, 64.50 mmol), m-Tolylamine (7.6 g, 70.95 mmol), Na₂CO₃ (26.0 g, 245.10 mmol) in 200 mL of ethanol was heated at 70 ° C. for 3 h. The reaction mixture was cooled and quenched with water (300 mL), then the solid was filtered off, washed with water and hexane to remove the brown color. The solid was partitioned between CH₂Cl₂ and water. The organic phase was washed with water then brine, dried over sodium sulfate and filtered. After concentrating under reduced pressure, a solid was obtained then recrystalized with CH₂Cl₂/hexane to afford 14.40 g of 1-(4-fluoro-phenyl)-2-m-tolylamino-ethanone. MS (ESI) 244.5.0 (M+H)⁺.

### Example 7

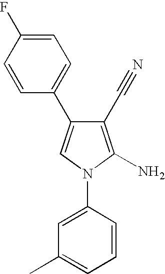

### Preparation of 2-Amino-4-(4-fluoro-phenyl)-1-m-tolyl-1H-pyrrole-3-carbonitrile

A solution of 21-(4-fluoro-phenyl)-2-m-tolylamino-ethanone (14.0 g, 57.55 mmol), malononitrile (4.35 mL, 69.06 mmol), NaOEt (21% wt, 30 mL, 74.82 mmol) in 100 mL of ethanol was heated at 80° C. for 2 h. The reaction mixture was cooled and concentrated under reduced pressure to remove ethanol. The residue was partitioned between EtOAc and water. The organic phase was washed with brine, dried over sodium sulfate and filtered. After concentrating under reduced pressure, the solvent was removed and the residue obtained was purified by silica gel chromatography to afford 16.75 g of 2-amino-4-(4-fluoro-phenyl)-1-m-tolyl-1H-pyrrole-3-carbonitrile. MS (ESI) 292.3 (M+H)⁺.

### Example 8

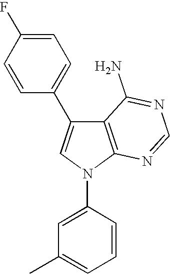

### Preparation of 5-(4-Fluoro-phenyl)-7-m-tolyl-7H-pyrrolo[2,3-d]pyrimidin-4-ylamine

The mixture of 2-amino-4-(4-fluoro-phenyl)-1-m-tolyl-1H-pyrrole-3-carbonitrile (1.00 g, 3.44 mmol) and formamide (3.00 mL, 75.6 mmol) was heated to 195° C. for 2 h in microwave reactor. The solvent was removed and the residue obtained was purified by silica gel chromatography to afford 0.88 g of 5-(4-fluoro-phenyl)-7-m-tolyl-7H-pyrrolo[2,3-d]pyrimidin-4-ylamine. MS (ESI) 319.6 (M+H)⁺

### Example 9

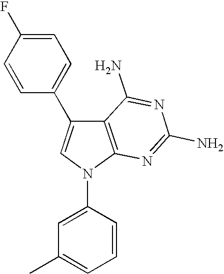

### Preparation of 5-(4-Fluoro-phenyl)-7-m-tolyl-7H-pyrrolo[2,3-d]pyrimidine-2,4-diamine

The mixture of 2-amino-4-(4-fluoro-phenyl)-1-m-tolyl-1H-pyrrole-3-carbonitrile (2.02 g, 6.94 mmol), aminonitrile (0.64 g, 15.27 mmol) and 4N HCl/dioxane (100 mL) was heated to reflux for o/n. After the reaction mixture was cooled to rt, aminonitrile (0.64 g, 15.27 mmol) and 4N HCl/dioxane (100 mL) were added and was heated to reflux for o/n. This process was repeated three time. The solvent was removed and NaOEt (21% wt, 100 mL, 27.76 mmol) was added and allowed to reflux for o/n. The precipitate was collected by filtration. The solid was partitioned between CH₂Cl₂ and water, the organics were washed with brine, dried over magnesium sulfate and concentrated. The residue obtained was purified by silica gel chromatography to afford 0.85 g of 5-(4-fluoro-phenyl)-7-m-tolyl-7H-pyrrolo[2,3-d]pyrimidine-2,4-diamine. MS (ESI) 334.4 (M+H)⁺

### Example 10

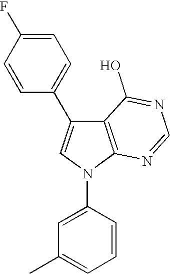

### Preparation of 5-(4-Fluoro-phenyl)-7-m-tolyl-7H-pyrrolo[2,3-d]pyrimidin-4-ol

The mixture of 2-amino-4-(4-fluoro-phenyl)-1-m-tolyl-1H-pyrrole-3-carbonitrile (6.64 g, 22.82 mmol) and formic acid (300 mL) was heated to reflux for o/n. Allowed the reaction mixture cool to rt and filtered off a solid which was washed with EtOH to afford 5.00 g of 5-(4-fluoro-phenyl)-7-m-tolyl-7H-pyrrolo[2,3-d]pyrimidin-4-ol. MS (ESI) 320.3 (M+H)⁺

### Example 11

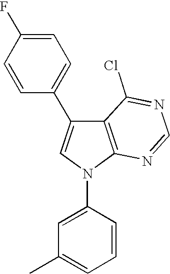

### Preparation of 4-Chloro-5-(4-fluoro-phenyl)-7-m-tolyl-7H-pyrrolo[2,3-d]pyrimidine

A round bottom flask was charged with 5-(4-fluoro-phenyl)-7-m-tolyl-7H-pyrrolo[2,3-d]pyrimidin-4-ol (4.50 g, 14.11 mmol), 7 mL of POCl₃ was added. This was heated to reflux for o/n. After the reaction mixture is cooled, poured into ice-H₂O (200 mL) and allowed to stir for 1 h. This was partitioned between CH2Cl12 and water. The CH₂Cl₂ layer was washed sat. NaHCO₃ with brine, dried over anhydrous magnesium sulfate, concentrated in vacuo, and purified by flash chromatography (gradient elution 5 to 10% ethyl acetate/hexanes) to yield 4-chloro-5-(4-fluoro-phenyl)-7-m-tolyl-7H-pyrrolo[2,3-d]pyrimidine. MS (ESI) 338.5 (M+H)⁺.

### Example 12

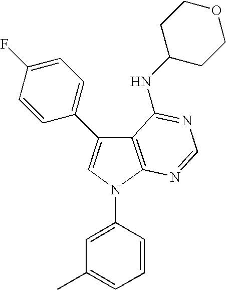

### Preparation of [5-(4-Fluoro-phenyl)-7-m-tolyl-7H-pyrrolo[2,3-d]pyrimidin-4-yl]-(tetrahydro-pyran-4-yl)-amine

The mixture of 4-chloro-5-(4-fluoro-phenyl)-7-m-tolyl-7H-pyrrolo[2,3-d]pyrimidine (0.17 g, 0.5 mmol), tetrahydro-pyran-4-ylamine (0.13 g, 1.3 mmol) in ethoxyethanol (10 mL) was microwaved at 150° C. for 2 h. The solvent was removed and the residue obtained was purified by silica gel chromatography (gradient elution 2%-10% methanol/dichloromethane) to afford 0.3 g of [5-(4-fluoro-phenyl)-7-m-tolyl-7H-pyrrolo[2,3-d]pyrimidin-4-yl]-(tetrahydro-pyran-4-yl)-amine. MS (ESI) 403.8 (M+H)⁺

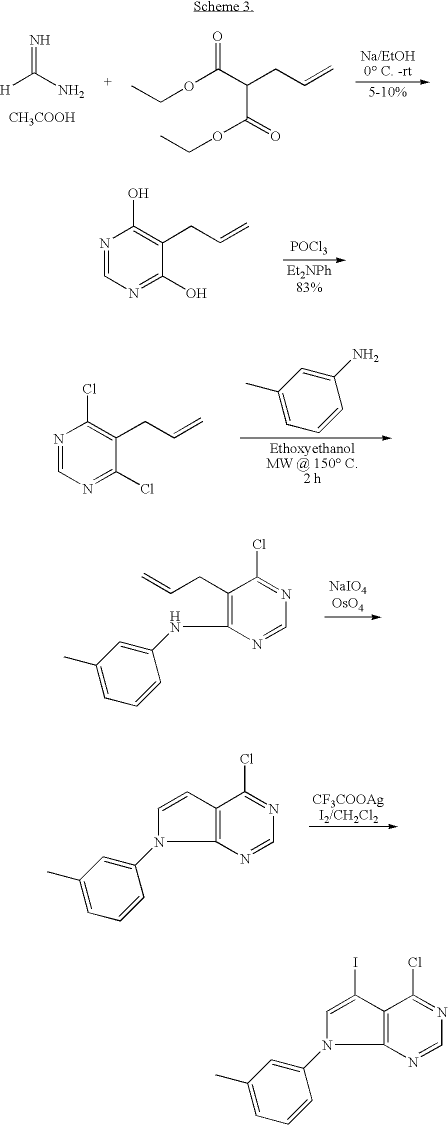

### Example 13

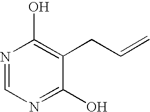

### Preparation of 5-Allyl-pyrimidine-4,6-diol

To a cold solution of EtOH (800 mL) at 0° C., was added sodium (47.00 g, 1.96 mol) portionwise. The mixture was allowed to stir at 0° C. until it became a homogeneous solution. To this solution at 0° C., was added formamidine acetic acid (63.62 g, 0.61 mol) slowly and stirred for an additional 40 min. Then a solution of 2-Allyl-malonic acid diethyl ester (100 mL, 0.56 mol) in EtOH (100 mL) was added slowly at 0° C. This was stirred at rt for 2 d. This was made acidic with conc. HCl (150 mL). The solid was filtered off and suspended in EtOAc, then refluxed for 30 min. The solid was collected by filtration and dried in vacuo in oven. to afford 117.54 g of 5-allyl-pyrimidine-4,6-diol. MS (ESI) 151.4 (M−H)⁻

### Example 14

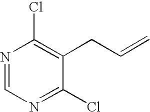

### Preparation of 5-Allyl-4,6-dichloro-pyrimidine

To a solution of 5-allyl-pyrimidine-4,6-diol (16.64 g, 109.4 mmol), Diethyl-phenyl-amine (17.5 mL, 131.28 mmol), Benzyl-triethyl-ammonium chloride (50.4 g, 228.3 mmol) in CH₃CN (270 mL), was added POCl₃ (54 mL, 108.0 mmol) slowly. This was heated at 110° C. for o/n under argon. After the mixture was cooled to rt, it was poured into crushed ice (500 g) and stirred for 30 min. This was extracted with EtOAc. The organic layer was washed with brine, dried over anhydrous magnesium sulfate, concentrated in vacuo, and purified by flash chromatography to yield 3.0 g of 5-allyl-4,6-dichloro-pyrimidine. MS (ESI) 190.2 (M+H)⁺.

### Example 15

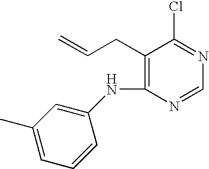

### Preparation of (5-Allyl-6-chloro-pyrimidin-4-yl)-m-tolyl-amine

The mixture of 5-allyl-4,6-dichloro-pyrimidine (1.7 g, 8.98 mmol), m-tolylamine (0.96 mL, 8.98 mmol) in ethoxyethanol (10 mL) was microwaved at 150° C. for 2 h. The solvent was removed and the residue obtained was purified by silica gel chromatography to afford 1.7 g of (5-allyl-6-chloro -pyrimidin-4-yl)-m-tolyl-amine. MS (ESI) 259.4 (M+H)⁺

### Example 16

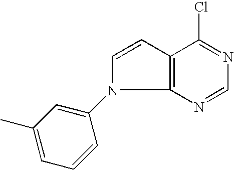

### Preparation of 4-Chloro-7-m-tolyl-7H-pyrrolo[2,3-d]pyrimidine

To a solution of (5-allyl-6-chloro-pyrimidin-4-yl)-m-tolyl-amine (1.8 g, 6.95 mmol) in acetone-H₂O (60 mL-10 mL), was added NaIO₄ (4.46 g, 20.85 mmol). The mixture was heated at 60° C. breiefly to obtain homogeneity, then a solution of 2.5% OsO4 in t-BuOH (0.5 mL, 7.14 mmol) was added. This was stirred at rt for 20 h, water (60 mL) and a sat. NaHSO₃ (60 mL) was added. The mixture was extracted with EtOAc. The organic layer was concentrated and the residue was dissolved in CH₂Cl₂ (50 mL), 4N HCl/dioxane (7 mL) was added and stirred for 4 h. The mixture was poured into sat. NaHCO₃, then extracted with CH₂Cl₂. The organics were washed with brine, dried over anhydrous magnesium sulfate, concentrated in vacuo, and purified by flash chromatography to yield 1.0 g of 4-chloro-7-m-tolyl-7H-pyrrolo[2,3-d]pyrimidine. MS (ESI) 244.6 (M+H)⁺.

### Example 17

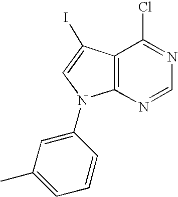

### Preparation of 4-Chloro-5-iodo-7-m-tolyl-7H-pyrrolo[2,3-d]pyrimidine

To a solution of 4-chloro-7-m-tolyl-7H-pyrrolo[2,3-d]pyrimidine (0.64 g, 2.63 mmol) and CF₃COOAg (0.93 g, 4.21 mmol) in CH₂Cl₂ (10 mL), was added a solution of I₂ (0.81 g, 3.16 mmol) in CH₂Cl₂ (10 mL) dropwise. This was stirred at rt for o/n. The solid was filtered off, the organic filtrate was concentrated in vacuo, and purified by flash chromatography to yield 0.80 g of 4-chloro-5-iodo-7-m-tolyl-7H-pyrrolo[2,3-d]pyrimidine. MS (ESI) 370.7 (M+H)⁺.

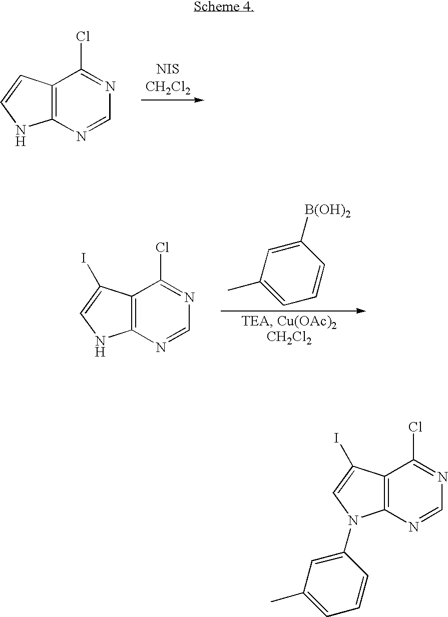

### Example 18

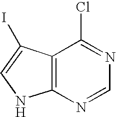

### Preparation of 4-Chloro-5-iodo-7H-pyrrolo[2,3-d]pyrimidine

To a solution of 4-chloro-7H-pyrrolo[2,3-d]pyrimidine (3.01 g, 20.18 mmol) in CH₂Cl₂ (200 mL), was added NIS (5.00 g, 22.20 mmol). The mixture was stirred for 30 min at rt. Filter off the off white solid and washed with water, dried in vacuum oven and recrystalized from MeOH to give 5.00 g of 4-chloro-5-iodo-7H-pyrrolo[2,3-d]pyrimidine. MS (ESI) 280.7 (M+H)⁺.

### Example 19

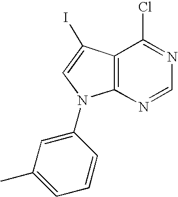

### Preparation of 4-Chloro-5-iodo-7-m-tolyl-7H-pyrrolo[2,3-d]pyrimidine

A round bottom flask was charged with 4-chloro-5-iodo-7H-pyrrolo[2,3-d]pyrimidine (2.00 g, 7.16 mmol), 3-methylbenzeneboronic acid (1.95 g, 14.34 mmol), triethyl amine (2.0 mL, 14.34 mmol), and copper acetate (1.30 g, 7.17 mmol). The vial was flushed with argon, 30 mL of CH₂Cl₂ was added, and the reaction mixture was stirred at rt for o/n. After filtration, the solvent was removed and the brown mass obtained was purified by silica gel chromatography to afford 2.1 g of 4-chloro-5-iodo-7-m-tolyl-7H-pyrrolo[2,3-d]pyrimidine. MS (ESI) 370.6 (M+H)⁺

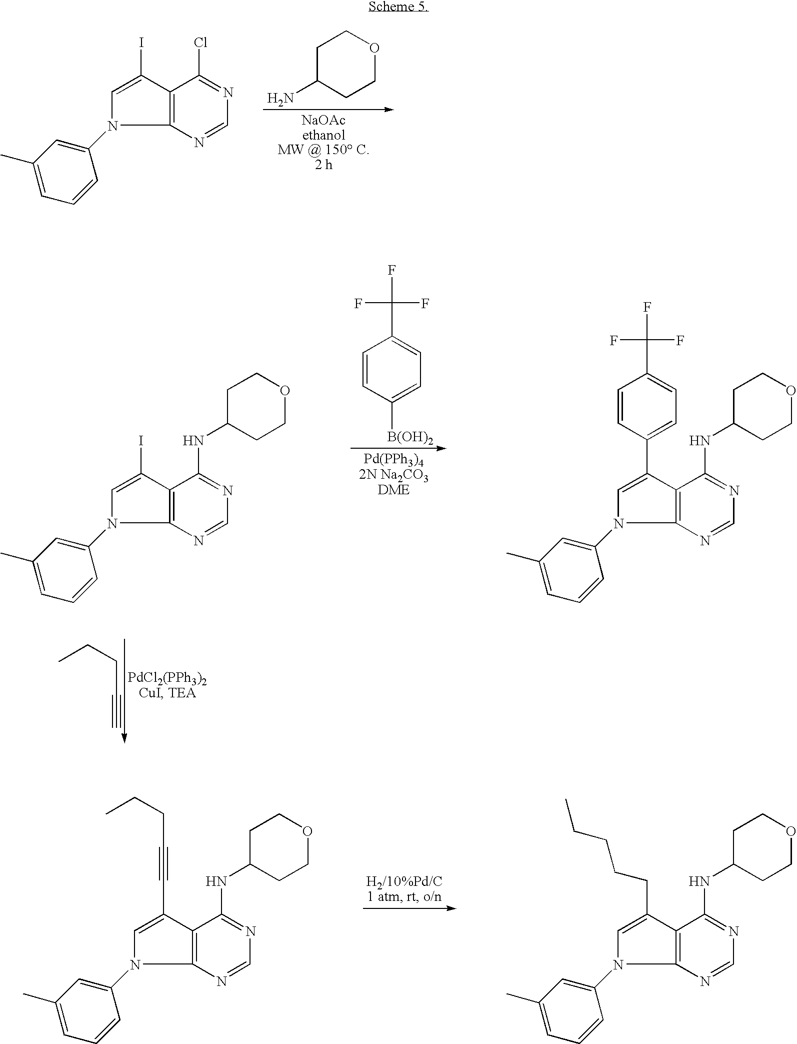

### Example 20

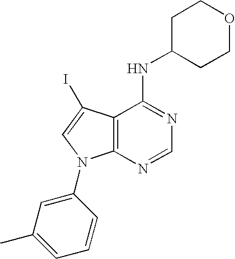

### Preparation of (5-Iodo-7-m-tolyl-7H-pyrrolo[2,3-d]pyrimidin-4-yl)-(tetrahydro-pyran-4-yl)-amine

The mixture of 4-chloro-5-iodo-7-m-tolyl-7H-pyrrolo[2,3-d]pyrimidine (0.08 g, 0.20 mmol), Tetrahydro-pyran-4-ylamine (0.04 g, 0.4 mmol) and NaOAc (0.1 g, 0.8 mmol) in ethanol (10 mL) was microwaved at 150° C. for 2 h. The solvent was removed and the residue obtained was purified by silica gel chromatography (gradient elution 5%-30% acetone/dichloromethane) to afford 0.08 g of (5-iodo-7-m-tolyl-7H-pyrrolo[2,3-d]pyrimidin-4-yl)-(tetrahydro-pyran-4-yl)-amine. MS (ESI) 434.5 (M+H)⁺

### Example 21

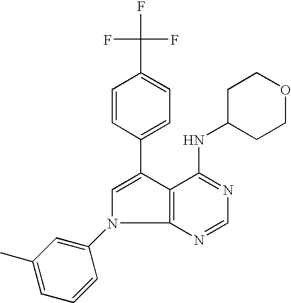

### Preparation of (Tetrahydro-pyran-4-yl)-[7-m-tolyl-5-(4-trifluoromethyl-phenyl)-7H-pyrrolo[2,3-d]pyrimidin-4-yl]-amine

The mixture of (5-iodo-7-m-tolyl-7H-pyrrolo[2,3-d]pyrimidin-4-yl)-(tetrahydro-pyran-4-yl)-amine (0.08 g, 0.18 mmol), 4-trifluorophenylboronic acid (0.04 g, 0.18 mmol), Pd(PPh₃)₄ (10 mg, 0.009 mmol), Na₂CO₃ (2N, 0.36 mL, 0.72 mmol) in DME (4 mL) was heated at 95° C. for o/n. After filtration, the solvent was removed and the residue obtained was purified by silica gel chromatography to afford 0.077 g of (tetrahydro-pyran-4-yl)-[7-m-tolyl-5-(4-trifluoromethyl-phenyl)-7H-pyrrolo[2,3-d]pyrimidin-4-yl]-amine. MS (ESI) 453.1 (M +H)⁺

### EXAMPLE 22

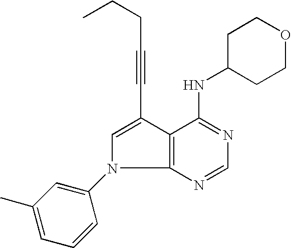

### Preparation of (5-Pent-1-ynyl-7-m-tolyl-7H-pyrrolo[2,3-d]pyrimidin-4-yl)-(tetrahydro-pyran-4-yl)-amine

The mixture of (5-iodo-7-m-tolyl-7H-pyrrolo[2,3-d]pyrimidin-4-yl)-(tetrahydro-pyran-4-yl)-amine (0.08 g, 0.18 mmol), Pent-1-yne (0.02 mL, 0.22 mmol), PdCl₂(PPh₃)₂ (0.3 mg, 0.004 mmol) and CuI (0.4 mg, 0.002 mmol) in TEA (4 mL) was heated at 55° C. for o/n. After filtration, the solvent was removed and the residue obtained was purified by silica gel chromatography (gradient elution 5-30% acetone/CH₂Cl₂) to afford 0.068 g of (5-pent-1-ynyl-7-m-tolyl-7H-pyrrolo[2,3-d]pyrimidin-4-yl)-(tetrahydro-pyran-4-yl)-amine. MS (ESI) 347.7 (M+H)⁺

### Example 23

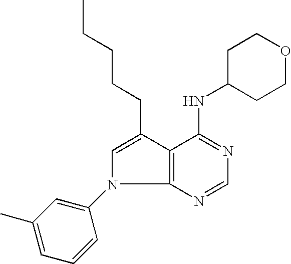

### Preparation of (5-Pentyl-7-m-tolyl-7H-pyrrolo[2,3-d]pyrimidin-4-yl)-(tetrahydro-pyran-4-yl)-amine

The mixture of (5-pent-1-ynyl-7-m-tolyl-7H-pyrrolo[2,3-d]pyrimidin-4-yl)-(tetrahydro-pyran-4-yl)-amine (0.04 g, 011 mmol), 10% Pd/C (0.005 g) in EtOH (5 mL) was hydrogenated at 1 atm and rt for o/n. After filtration through a pre-pad celite, the solvent was removed to afford 0.025 g of (5-pentyl-7-m-tolyl-7H-pyrrolo[2,3-d]pyrimidin-4-yl)-(tetrahydro-pyran-4-yl)-amine. MS (ESI) 378.9 (M+H)⁺

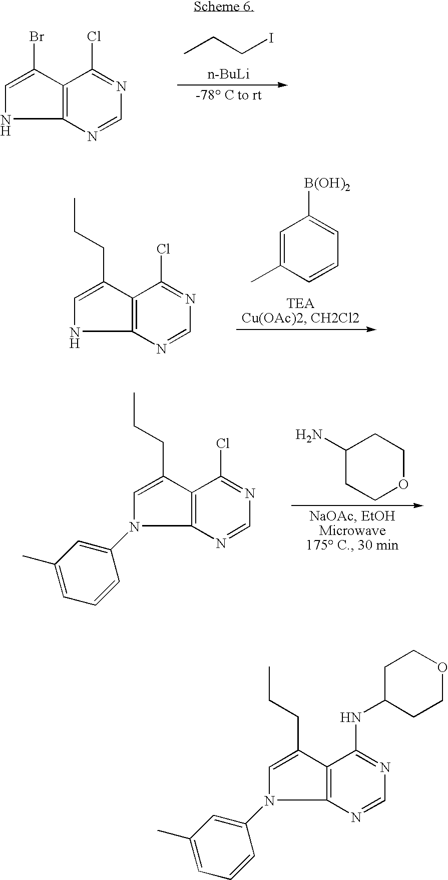

### Example 24

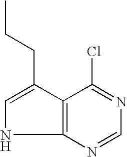

### Preparation of 4-Chloro-5-propyl-7H-pyrrolo[2,3-d]pyrimidine

To a solution of 5-bromo-4-chloro-7H-pyrrolo[2,3-d]pyrimidine (1.00 g, 4.30 mmol) in THF (10 mL) at −78° C., was added n-BuLi (2.5M/hexane, 4.0 mL, 9.9 mmol) dropwise. This was stirred at −78° C. for 40 min. BuI (0.8 mL, 4.30 mmol) was added and allow the reaction mixture to warm to rt slowly for o/n. The reaction was quenched with H₂O (2 mL). The reaction mixture was concentrated to remove THF and partitioned between EtOAc and H₂O. The organic layer was washed with H2O and brine, concentrated in vacuo, and purified by flash chromatography to yield 0.50 g of 4-chloro-5-propyl-7H-pyrrolo[2,3-d]pyrimidine. MS (ESI) 210.3 (M+H)⁺.

### Example 25

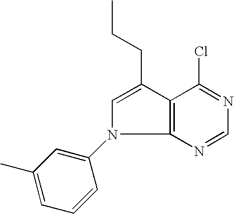

### Preparation of 4-Chloro-5-propyl-7-m-tolyl-7H-pyrrolo[2,3-d]pyrimidine

A round bottom flask was charged with 4-chloro-5-propyl-7H-pyrrolo[2,3-d]pyrimidine (0.48 g, 2.3 mmol), 3-methylbenzeneboronic acid (0.62 g, 4.6 mmol), triethyl amine (0.65 mL, 4.6 mmol), and copper acetate (0.42 g, 2.30 mmol). The vial was flushed with argon, 30 mL of CH₂Cl₂ was added, and the reaction mixture was stirred at rt for o/n. After filtration, the solvent was removed and the brown mass obtained was purified by silica gel chromatography to afford 0.50 g of 4-chloro-5-propyl-7-m-tolyl-7H-pyrrolo[2,3-d]pyrimidine. MS (ESI) 300.2 (M+H)⁺

### Example 26

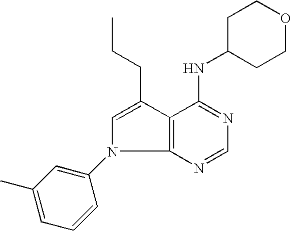

### Preparation of (5-Propyl-7-m-tolyl-7H-pyrrolo[2,3-d]pyrimidin-4-yl)-(tetrahydro-pyran-4-yl)-amine

The mixture of 4-chloro-5-propyl-7-m-tolyl-7H-pyrrolo[2,3-d]pyrimidine (0.10 g, 0.03 mmol), tetrahydro-pyran-4-ylamine (0.065 g, 0.64 mmol) and NaOAc (0.24 g, 1.74 mmol) in ethanol (10 mL) was microwaved at 170° C. for 30 min. The solvent was removed and the residue obtained was purified by silica gel chromatography (gradient elution 5-30% acetone/dichloromethane) to afford 0.06 g of (5-propyl-7-m-tolyl-7H-pyrrolo[2,3-d]pyrimidin-4-yl)-(tetrahydro-pyran-4-yl)-amine. MS (ESI) 365.8 (M+H)⁺

### Example 27

### Formulations

Pharmaceutical preparations for delivery by various routes are formulated as shown in the following Tables. “Active ingredient” or “Active compound” as used in the Tables means one or more of the Compounds of Formula I.

Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation

| Ingredient | % wt./wt. |
|---|---|
| Active ingredient | 0.25 g |
| Sodium Chloride | qs to make isotonic |
| Water for injection | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation

| Ingredients | Grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

### Nasal Spray Formulations

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 2-4 sprays every 4-12 hours.

### Example 28

### P2X₃/P2X_{2/3} FLIPR (Fluorometric Imaging Plate Reader) Assay

CHO-K1 cells were transfected with cloned rat P2X₃ or human P2X_{2/3} receptor subunits and passaged in flasks. 18-24 hours before the FLIPR experiment, cells were released from their flasks, centrifuged, and resuspended in nutrient medium at 2.5×10⁵ cells/ml. The cells were aliquoted into black-walled 96-well plates at a density of 50,000 cells/well and incubated overnight in 5% CO₂ at 37° C. On the day of the experiment, cells were washed in FLIPR buffer (calcium- and magnesium-free Hank's balanced salt solution, 10 mM HEPES, 2 mM CaCl₂, 2.5 mM probenecid; FB). Each well received 100 μl FB and 100 μl of the fluorescent dye Fluo-3 AM [2 μM final conc.]. After a 1 hour dye loading incubation at 37° C., the cells were washed 4 times with FB, and a final 75 μl/well FB was left in each well.

Test compounds (dissolved in DMSO at 10 mM and serially diluted with FB) or vehicle were added to each well (25 μl of a 4× solution) and allowed to equilibrate for 20 minutes at room temperature. The plates were then placed in the FLIPR and a baseline fluorescence measurement (excitation at 488 nm and emission at 510-570 nm) was obtained for 10 seconds before a 100 μl/well agonist or vehicle addition. The agonist was a 2× solution of α,β-meATP producing a final concentration of 1 μM (P2X₃) or 5 μM (P2X_{2/3}). Fluorescence was measured for an additional 2 minutes at 1 second intervals after agonist addition. A final addition of ionomycin (5 μM, final concentration) was made to each well of the FLIPR test plate to establish cell viability and maximum fluorescence of dye-bound cytosolic calcium. Peak fluorescence in response to the addition of a,(3-meATP (in the absence and presence of test compounds) was measured and inhibition curves generated using nonlinear regression. PPADS, a standard P2X antagonist, was used as a positive control.

Using the above assay, the compounds of Table 1 were all determined to be active for the P2X3 receptor. The compounds have an IC50 between about 5.0 and about 6.6 for P2X3. Many of the compounds have an IC50 of between about 6.0 and about 6.6 for P2X3. For example, [5-(4-Fluoro-phenyl)-7-m-tolyl-7H-pyrrolo[2,3-d]pyrimidin-4-yl]-(tetrahydro-pyran-4-yl)-amine exhibited an IC50 of about 6.51.

Using the above assay, most of the compounds of Table 1 were determined to be active for the P2X2/3 receptor. The compounds have an IC50 between about 5.0 and about 6.3 for P2X2/3. Many of the compounds have an IC50 of between about 5.5 and about 6.3 for P2X2/3. For example, (R)-2-[7-(3-Chloro-4-methyl-phenyl)-5-phenyl-7H-pyrrolo[2,3-d]pyrimidin-4-ylamino]-propan-1-ol exhibited and IC50 of about 6.3.

### Example 29

### In Vivo Assay for Asthma and Lung Function

BALb/cJ mice are immunized with a standard immunization protocol. Briefly, mice (N=8/group) are immunized i.p. with ovalbumin (OVA; 10 μg) in alum on days 0 and 14. Mice are then challenged with aerosolized OVA (5%) on day 21 and 22. Animals receive vehicle (p.o.) or a compound of the invention (100 mg/kg p.o.) all starting on day 20.

Lung function is evaluated on day 23 using the Buxco system to measure PenH in response to an aerosol methacholine challenge. Mice are then euthanized and plasma samples collected at the end of the study.

### Example 30

### Volume Induced Bladder Contraction Assay

Female Sprague-Dawley rats (200-300 g) were anesthetized with urethane (1.5 g/kg, sc). The animals were tracheotomized, and a carotid artery and femoral vein were cannulated for blood pressure measurement and drug administration, respectively. A laparotomy was performed and the ureters were ligated and transected proximal to the ligation. The external urethral meatus was ligated with silk suture and the urinary bladder was cannulated via the dome for saline infusion and bladder pressure measurement.

Following a 15-30 minute stabilization period the bladder was infused with room temperature saline at 100 μl/min until continuous volume-induced bladder contractions (VIBCs) were observed. The infusion rate was then lowered to 3-5 μl/min for 30 minutes before the bladder was drained and allowed to rest for 30 minutes. All subsequent infusions were performed as indicated except the lower infusion rate was maintained for only 15 minutes instead of 30 minutes. Bladder filling and draining cycles were repeated until the threshold volumes (TV; the volume needed to trigger the first micturition bladder contraction) varied by less than 10% for two consecutive baselines and contraction frequency was within 2 contractions for a 10 minute period following the slower infusion rate. Once reproducible TVs and VIBCs were established the bladder was drained and the animal was dosed with drug or vehicle (0.5 ml/kg, i.v.) 3 minutes prior to the start of the next scheduled infusion.

### Example 31

### Formalin Pain Assay

Male Sprague Dawley rats (180-220 g) are placed in individual Plexiglas cylinders and allowed to acclimate to the testing environment for 30 min. Vehicle, drug or positive control (morphine 2 mg/kg) is administered subcutaneously at 5 ml/kg. 15 min post dosing, formalin (5% in 50 μl) is injected into plantar surface of the right hind paw using a 26-gauge needle. Rats are immediately put back to the observation chamber. Mirrors placed around the chamber allow unhindered observation of the formalin-injected paw. The duration of nociphensive behavior of each animal is recorded by a blinded observer using an automated behavioral timer. Hindpaw licking and shaking/lifting are recorded separately in 5 min bin, for a total of 60 min. The sum of time spent licking or shaking in seconds from time 0 to 5 min is considered the early phase, whereas the late phase is taken as the sum of seconds spent licking or shaking from 15 to 40 min. A plasma sample is collected.

### Example 32

### Colon Pain Assay

Adult male Sprague-Dawley rats (350-425 g; Harlan, Indianapolis, Ind.) are housed 1-2 per cage in an animal care facility. Rats are deeply anesthetized with pentobarbital sodium (45 mg/kg) administered intraperitoneally. Electrodes are placed and secured into the external oblique musculature for electromyographic (EMG) recording. Electrode leads are tunneled subcutaneously and exteriorized at the nape of the neck for future access. After surgery, rats are housed separately and allowed to recuperate for 4-5 days prior to testing.

The descending colon and rectum are distended by pressure-controlled inflation of a 7-8 cm-long flexible latex balloon tied around a flexible tube. The balloon is lubricated, inserted into the colon via the anus, and anchored by taping the balloon catheter to the base of the tail. Colorectal distension (CRD) is achieved by opening a solenoid gate to a constant pressure air reservoir. Intracolonic pressure is controlled and continuously monitored by a pressure control device. Response is quantified as the visceromotor response (VMR), a contraction of the abdominal and hindlimb musculature. EMG activity produced by contraction of the external oblique musculature is quantified using Spike2 software (Cambridge Electronic Design). Each distension trial lasts 60 sec, and EMG activity is quantified for 20 sec before distension (baseline), during 20 sec distension, and 20 sec after distention. The increase in total number of recorded counts during distension above baseline is defined as the response. Stable baseline responses to CRD (10, 20, 40 and 80 mmHg, 20 seconds, 4 minutes apart) are obtained in conscious, unsedated rats before any treatment.

Compounds are evaluated for effects on responses to colon distension initially in a model of acute visceral nociception and a model of colon hypersensitivity produced by intracolonic treatment with zymosan (1 mL, 25 mg/mL) instilled into the colon with a gavage needle inserted to a depth of about 6 cm. Experimental groups will consist of 8 rats each.

Acute visceral nociception: For testing effects of drug on acute visceral nociception, 1 of 3 doses of drug, vehicle or positive control (morphine, 2.5 mg/kg) are administered after baseline responses are established; responses to distension are followed over the next 60-90 minutes.

Visceral hypersensitivity: For testing effects of drug or vehicle after intracolonic treatment with zymosan, intracolonic treatment is given after baseline responses are established. Prior to drug testing at 4 hours, responses to distension are assessed to establish the presence of hypersensitivity. In zymosan-treated rats, administration of 1 of 3 doses of drug, vehicle or positive control (morphine, 2.5 mg/kg) are given 4 hours after zymosan treatment and responses to distension followed over the next 60-90 minutes.

### Example 33

### Cold Allodynia in Rats with a Chronic Constriction Injury of the Sciatic Nerve

The effects of compounds of this invention on cold allodynia are determined using the chronic constriction injury (CCI) model of neuropathic pain in rats, where cold allodynia is measured in a cold-water bath with a metal-plate floor and water at a depth of 1.5-2.0 cm and a temperature of 3-4° C. (Gogas, K. R. et al., Analgesia, 1997, 3, 1-8).

Specifically, CCI, rats are anesthetized; the trifurcation of the sciatic nerve is located and 4 ligatures (4-0, or 5-0 chromic gut) are placed circumferentially around the sciatic nerve proximal to the trifurcation. The rats are then allowed to recover from the surgery. On days 4-7 after surgery, the rats are initially assessed for cold -induced allodynia by individually placing the animals in the cold-water bath and recording the total lifts of the injured paw during a 1-min period of time: The injured paw is lifted out of the water. Paw lifts associated with locomotion or body repositioning are not recorded. Rats that displayed 5 lifts per min or more on day 4-7 following surgery are considered to exhibit cold allodynia and are used in subsequent studies. In the acute studies, vehicle, reference compound or compounds of this invention are administered subcutaneously (s.c.) 30 min before testing. The effects of repeated administration of the compounds of this invention on cold allodynia are determined 14, 20 or 38 h following the last oral dose of the following regimen: oral (p.o.) administration of vehicle, reference or a compound of this invention at ˜12 h intervals (BID) for 7 days.

### Example 34

### Cancer Bone Pain in C3H/HeJ Mice

The effects of compounds of this invention on bone pain are determined between Day 7 to Day 18 following intramedullary injection of 2472 sarcoma cells into the distal femur of C3H/HeJ mice.

Specifically, NCTC 2472 tumor cells (American Type Culture Collection, ATCC), previously shown to form lytic lesions in bone after intramedullary injection, are grown and maintained according to ATCC recommendations. Approximately 10⁵ cells are injected directly into the medullary cavity of the distal femur in anesthetized C3H/HeJ mice. Beginning on about Day 7, the mice are assessed for spontaneous nocifensive behaviors (flinching & guarding), palpation-evoked nocifensive behaviors (flinching & guarding), forced ambultory guarding and limb use. The effects of compounds of this invention are determined following a single acute (s.c.) administration on Day 7-Day 15. In addition, the effects of repeated (BID) administration of compounds of this invention from Day 7-Day 15 are determined within 1 hour of the first dose on Days 7, 9, 11, 13 and 15.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed is:

1. A compound of Formula I 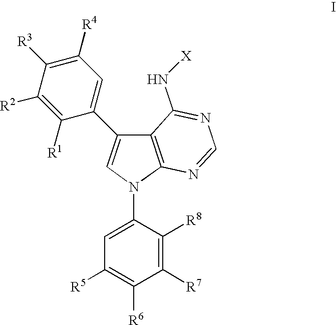
wherein:
X is CX^{1a}X^{1b}CHX^{1c}OX^{1d};
X^{1a} and X^{1b} are independently H, lower alkyl, or lower hydroxy alkyl;
X^{1b} is H, lower alkyl, lower alkoxy, lower hydroxyalkyl, or hydroxy lower alkoxy; or X^{1b} and X^{1c} together form tetrahydrofuranyl;
X^{1d} is H, lower alkyl, or lower alkoxy;
each of R¹, R², R³, and R⁴ is independently H, halogen, lower alkyl, hydroxy, lower alkoxy, amido, sulfonamido, or lower haloalkyl; and
each of R⁵, R⁶, R⁷, and R⁸ is independently H, halogen, lower alkyl, lower alkoxy, heterocycloalkyl, or lower haloalkyl;
with the proviso that R¹, R², R³, and R⁴ are not all H.

2. The compound of claim 1, wherein X^{1d} is H.

3. The compound of claim 1, wherein X^{1d} is methyl.

4. A compound of Formula II 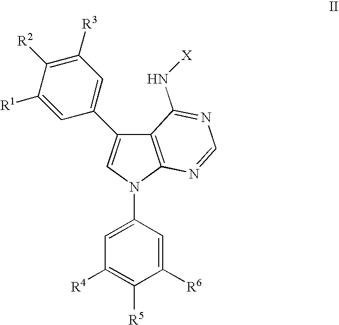
wherein:
X is CHX^{1a}CHX^{1b}CHX^{1c}OX^{1d};
X^{1a} is H, lower alkyl, or lower hydroxy alkyl;
X^{1b} is H, H lower alkyl, lower alkoxy, lower hydroxyalkyl, or hydroxy lower alkoxy;
X^{1c} is H, lower alkyl, lower alkoxy, lower hydroxyalkyl, or hydroxy lower alkoxy;
X^{1d} is H, lower alkyl, lower alkoxy, lower haloalkyl, or lower hydroxyalkyl; or X^{1c} and X^{1d} together form tetrahydrofuranyl;
each of R¹, R², and R³ is independently H, halogen, lower alkyl, hydroxy, lower alkoxy, amido, sulfonamido, or lower haloalkyl; and
each of R⁴, R⁵, and R⁶ is independently H, halogen, lower alkyl, lower alkoxy, heterocycloalkyl, or lower haloalkyl;
with the proviso that each of R¹, R², and R³ are not H.

5. The compound of claim 4, wherein X^{1c} is H.

6. The compound of claim 4, wherein X^{1c} is methyl.

7. A compound of Formula III 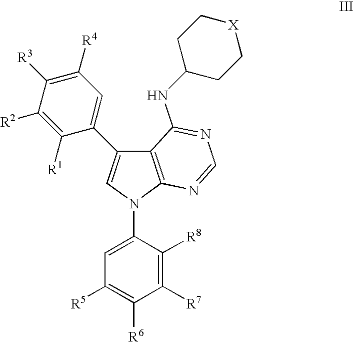
wherein:
X is NX′ or CH(OH);
X′ is H or lower alkyl;
each of R¹, R², R³, and R⁴ is independently H, halogen, lower alkyl, hydroxy, lower alkoxy, lower thioalkyl, amido, sulfonamido, or lower haloalkyl; and
each of R⁵, R⁶, R⁷, and R⁸ is independently H, halogen, lower alkyl, lower alkoxy, heterocycloalkyl, or lower haloalkyl.

8. A compound of Formula IV 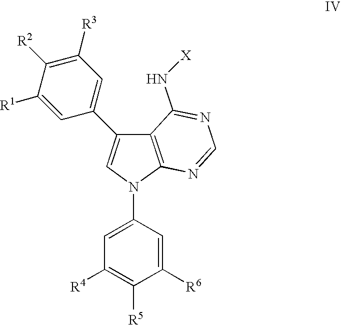
wherein:
X is CH₂CH₂CH₂OX¹;
X¹ is H, lower alkyl, lower haloalkyl, or lower hydroxy alkyl;
each of R¹, R², and R³ is independently H, halogen, lower alkyl, hydroxy, lower alkoxy, amido, sulfonamido, or lower haloalkyl; and
each of R⁴, R⁵, and R⁶ is independently H, halogen, lower alkyl, lower alkoxy, heterocycloalkyl, or lower haloalkyl;
with the proviso that when each of R¹, R², and R³ are H, X¹ is not H, methyl, ethyl or isopropyl.

9. A compound of Formula V 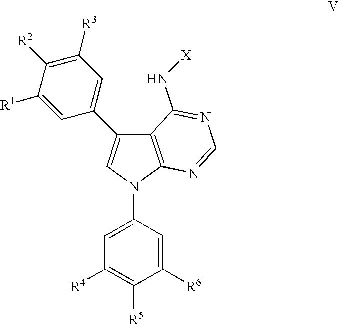
wherein:
X is CH₂CH₂X¹;
X¹ is heterocycloalkyl or heteroaryl, optionally substituted with one or more X^{1′}; X^{1′} is lower alkyl or oxo;
each of R¹, R², and R³ is independently H, halogen, lower alkyl, hydroxy, lower alkoxy, amido, sulfonamido, or lower haloalkyl; and
each of R⁴, R⁵, and R⁶ is independently H, halogen, lower alkyl, lower alkoxy, heterocycloalkyl, or lower haloalkyl; and
with the proviso that when each of R¹, R², and R³ are H, X¹ is not morpholine.

10. A compound of Formula VI 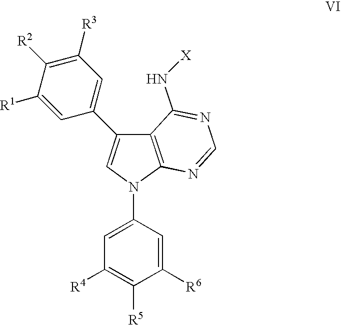
wherein:
X is CH₂CH₂CH₂X¹;
X¹ is heterocycloalkyl or heteroaryl, optionally substituted with one or more X^{1′}; X^{1′} is lower alkyl or oxo;
each of R¹, R², and R³ is independently H, halogen, lower alkyl, hydroxy, lower alkoxy, amido, sulfonamido, or lower haloalkyl; and
each of R⁴, R⁵, and R⁶ is independently H, halogen, lower alkyl, lower alkoxy, heterocycloalkyl, or lower haloalkyl;
with the proviso that when each of R¹, R², and R³ are H, X^{l} is not morpholine, pyrrolidin-2-one, or imidazole.

11. A compound of Formula VII 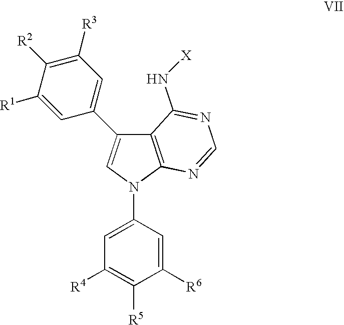
wherein:
X is C(═O)X¹;
X¹ is lower alkyl or lower alkoxy;
each of R¹, R², and R³ is independently H, halogen, lower alkyl, hydroxy, lower alkoxy, amido, sulfonamido, or lower haloalkyl; and
each of R⁴, R⁵, and R⁶ is independently H, halogen, lower alkyl, lower alkoxy, heterocycloalkyl, or lower haloalkyl.

12. A compound of Formula VIII 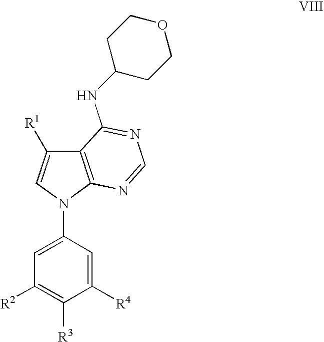
wherein:
R¹ is H, halogen, hydroxy, or R′;
R′ is lower alkyl, phenyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
each of R², R³, and R⁴ is independently H, halogen, lower alkyl, lower alkoxy, heterocycloalkyl, or lower haloalkyl.

13. A compound of Formula IX 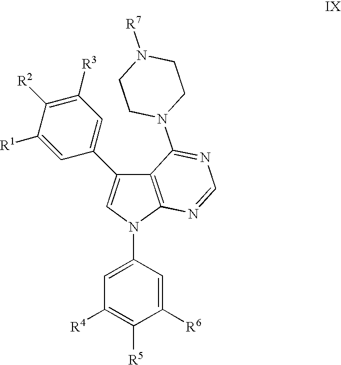
wherein:
each of R¹, R², and R³ is independently H, halogen, lower alkyl, hydroxy, lower alkoxy, amido, sulfonamido, or lower haloalkyl; and
each of R⁴, R⁵, and R⁶ is independently H, halogen, lower alkyl, lower alkoxy, heterocycloalkyl, or lower haloalkyl; and
R⁷ is lower alkyl, lower alkoxy, or lower hydroxyalkyl.

14. A compound of Formula X 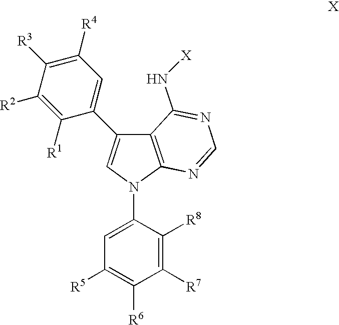
wherein:
X is CHX^{1a}CHX^{1b}OX^{1c};
X^{1a} and X^{1b} are each independently H, lower alkyl, hydroxy, lower alkoxy, or lower hydroxy alkyl;
X^{1c} is H, lower alkyl, lower hydroxyalkyl, or lower alkoxy;
each of R¹, R², R³, and R⁴ is independently H, halogen, lower alkyl, hydroxy, lower alkoxy, amido, sulfonamido, or lower haloalkyl; and
each of R⁵, R⁶, R⁷, and R⁸ is independently H, halogen, lower alkyl, lower alkoxy, heterocycloalkyl, or lower haloalkyl;
with the proviso that when each of R¹, R², R³, and R⁴ are H and X^{1c} is H, than either X^{1a} is methyl, isopropyl, or lower hydroxyalkyl; and
with the further proviso that when each of R¹, R², R³, and R⁴ are H and X^{1c} is methyl, then either R⁵ or R⁷ is fluoro, lower alkoxy, or tert-butyl; or both R⁵ and R⁷ are not H; or R⁶ is lower haloalkyl or heterocycloalkyl; or R⁷ is methyl and R⁶ is halogen or lower alkoxy; or R⁸ is fluoro;
or X^{1a} is lower alkoxy or lower hydroxyalkyl.

15. A compound of Formula XI 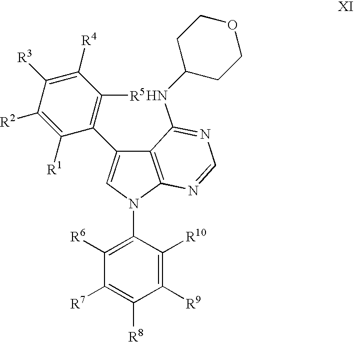
wherein:
each of R^{l}, R², R³, R⁴, and R⁵ is independently H, halogen, lower alkyl, lower alkenyl, hydroxy, amino, lower hydroxyalkyl, lower alkoxy, lower thioalkyl, amido, sulfonamido, lower alkyl sulfonyl, lower haloalkyl, lower haloalkoxy, cyano, or —C(═O)R′;
R′ is heterocycloalkyl, lower alkyl, lower alkoxy, hydroxy; and
each of R⁶, R⁷, R⁸, R⁹, and R¹⁰ is independently H, halogen, lower alkyl, lower alkoxy, heterocycloalkyl, or lower haloalkyl.

16. A compound of Formula XII 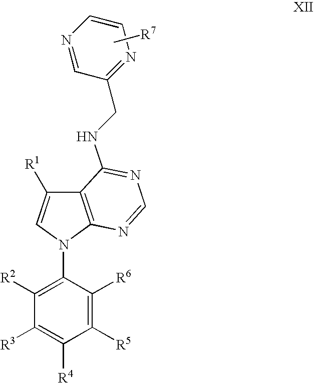
wherein:
R¹ is H, halogen, hydroxy, or R′;
R′ is lower alkyl, phenyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
each of R², R³, R⁴ R⁵, R⁶, and R⁷ is independently H, halogen, lower alkyl, lower alkoxy, heterocycloalkyl, or lower haloalkyl.

17. A compound of Formula XIII 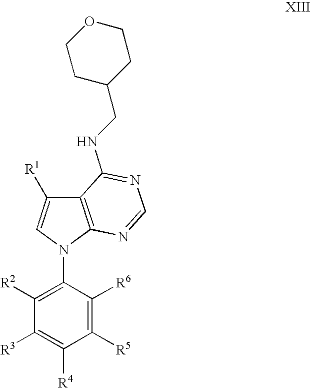
wherein:
R¹ is H, halogen, hydroxy, or R′;
R′ is lower alkyl, phenyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
each of R², R³, R⁴ R⁵, R⁶, and R⁷ is independently H, halogen, lower alkyl, lower alkoxy, heterocycloalkyl, or lower haloalkyl.

18. A compound of Formula XIV 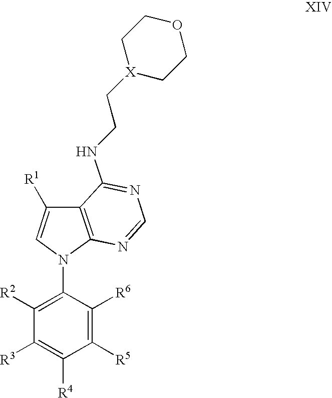
wherein:
X is CH or N;
R¹ is H, halogen, hydroxy, or R′;
R′ is lower alkyl, phenyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R″;
R″ is lower alkyl, halogen, hydroxy, lower alkoxy, amino, lower haloalkyl, or lower hydroxyalkyl; and
each of R², R³, R⁴ R⁵, R⁶, and R⁷ is independently H, halogen, lower alkyl, lower alkoxy, heterocycloalkyl, or lower haloalkyl.

19. A compound of Formula XV 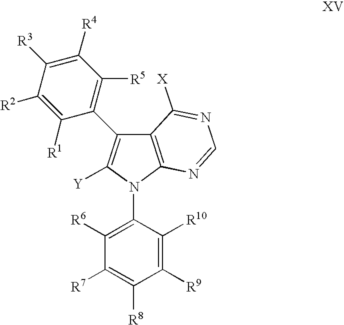
wherein:
X and Y are independently selected from H, halogen, hydroxy, and X′;
X′ is lower alkyl, phenyl, lower alkoxy, lower haloalkyl, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more X″;
X″ is lower alkyl, halogen, hydroxy, lower alkoxy, amino, lower haloalkyl, or lower hydroxyalkyl;
R³ is halogen;
each of R¹, R², R⁴, and R⁵ is independently H, halogen, lower alkyl, lower alkenyl, hydroxy, amino, lower hydroxyalkyl, lower alkoxy, lower thioalkyl, amido, sulfonamido, lower alkyl sulfonyl, lower haloalkyl, lower haloalkoxy, cyano, or —C(═O)R′;
R′ is heterocycloalkyl, lower alkyl, lower alkoxy, hydroxy;
R⁷ is F, Br, I, hydroxy, or R⁷;
R^{7′} is lower alkyl, lower alkoxy, lower haloalkyl, hydroxy, lower hydroxyalkyl, lower alkenyl, lower alkynyl, phenyl lower alkynyl, lower alkoxy, cycloalkenyl, lower alkyl phenylalkyl, aryl, heteroaryl, or alkylheteroaryl, optionally substituted with one or more R^{7″};
R^{7″} is lower alkyl, halogen, hydroxy, lower alkoxy, amino, lower haloalkyl, or lower hydroxyalkyl; and
each of R⁶, R⁸, R⁹, and R¹⁰ is independently H, halogen, lower alkyl, lower alkoxy, heterocycloalkyl, or lower haloalkyl.

20. A compound selected from the group consisting of:
(5,7-Diphenyl-7H-pyrrolo[2,3-d]pyrimidin-4-yl)-(2-morpholin-4-yl-ethyl)-amine;
(2-Morpholin-4-yl-ethyl)-(5-phenyl-7-m-tolyl-7H-pyrrolo[2,3-d]pyrimidin-4-yl)-amine;
5-(4-Fluoro-phenyl)-7-m-tolyl-7H-pyrrolo[2,3-d]pyrimidin-4-ylamine;
5-(4-Fluoro-phenyl)-7-m-tolyl-7H-pyrrolo[2,3-d]pyrimidine-2,4-diamine;
5-(4-Fluoro-phenyl)-7-m-tolyl-7H-pyrrolo[2,3-d]pyrimidine;
Cyclopentyl-[5-(4-fluoro-phenyl)-7-m-tolyl-7H-pyrrolo[2,3-d]pyrimidin-4-yl]-amine;
tert-Butyl-[5-(4-fluoro-phenyl)-7-m-tolyl-7H-pyrrolo[2,3-d]pyrimidin-4-yl]-amine;
2-[5-(4-Fluoro-phenyl)-7-m-tolyl-7H-pyrrolo[2,3-d]pyrimidin-4-ylamino]-2-methyl-propan-1-ol;
5-(4-Fluoro-phenyl)-7-m-tolyl-7H-pyrrolo[2,3-d]pyrimidin-4-ol;
5-(4-Fluoro-phenyl)-4-(2-methoxy-ethoxy)-7-m-tolyl-7H-pyrrolo[2,3-d]pyrimidine;
[5-(4-Fluoro-phenyl)-7-m-tolyl-7H-pyrrolo[2,3-d]pyrimidin-4-yl]-(tetrahydro-pyran-4-yl)-amine;
(R)-2-[7-(3-Chloro-4-methyl-phenyl)-5-phenyl-7H-pyrrolo[2,3-d]pyrimidin-4-ylamino]-propan-1-ol;
2-[5-(4-Fluoro-phenyl)-7-m-tolyl-7H-pyrrolo[2,3-d]pyrimidin-4-ylamino]-2-methyl-propane-1,3-diol;
2-[7-(3-Chloro-4-methyl-phenyl)-5-phenyl-7H-pyrrolo[2,3-d]pyrimidin-4-ylamino]-propane-1,3-diol;
(2-Methoxy-ethyl)-(7-m-tolyl-5-p-tolyl-7H-pyrrolo[2,3-d]pyrimidin-4-yl)-amine;
N-[7-(3-Chloro-4-methyl-phenyl)-5-phenyl-7H-pyrrolo[2,3-d]pyrimidin-4-yl]-2-methoxy-acetamide;
[5-(4-Fluoro-phenyl)-7-m-tolyl-7H-pyrrolo[2,3-d]pyrimidin-4-yl]-(2-methoxy-ethyl)-amine;
[7-(3-Chloro-4-methyl-phenyl)-5-phenyl-7H-pyrrolo[2,3-d]pyrimidin-4-yl]-(tetrahydro-pyran-4-yl)-amine;
[5-(4-Fluoro-phenyl)-7-m-tolyl-7H-pyrrolo[2,3-d]pyrimidin-4-yl]-(2-methoxy-1-methoxymethyl- ethyl)-amine;
2-[7-(3-Chloro-4-methyl-phenyl)-5-phenyl-7H-pyrrolo[2,3-d]pyrimidin-4-ylamino]-3-methyl-butan-1-ol;
N-[7-(3-Chloro-4-methyl-phenyl)-5-phenyl-7H-pyrrolo[2,3-d]pyrimidin-4-yl]-propionamide;
(S)-2-[7-(3-Chloro-4-methyl-phenyl)-5-phenyl-7H-pyrrolo[2,3-d]pyrimidin-4-ylamino]-3-methyl-butan-1-ol;
2-[5-(4-Fluoro-phenyl)-7-m-tolyl-7H-pyrrolo[2,3-d]pyrimidin-4-ylamino]-2-methyl-propan-1-ol;
(5-Pent-1-ynyl-7-m-tolyl-7H-pyrrolo[2,3-d]pyrimidin-4-yl)-(tetrahydro-pyran-4-yl)-amine;
(5-Ethynyl-7-m-tolyl-7H-pyrrolo[2,3-d]pyrimidin-4-yl)-(tetrahydro-pyran-4-yl)-amine;
(5-Pentyl-7-m-tolyl-7H-pyrrolo[2,3-d]pyrimidin-4-yl)-(tetrahydro-pyran-4-yl)-amine;
[5-(4-Fluoro-3-methyl-phenyl)-7-m-tolyl-7H-pyrrolo[2,3-d]pyrimidin-4-yl]-(tetrahydro-pyran-4-yl)-amine;
(R)-2-[7-(3-Chloro-4-methyl-phenyl)-5-phenyl-7H-pyrrolo[2,3-d]pyrimidin-4-ylamino]-3-methyl-butan-1-ol;
[5-(4-Isopropoxy-phenyl)-7-m-tolyl-7H-pyrrolo[2,3-d]pyrimidin-4-yl]-(tetrahydro-pyran-4-yl)-amine;
(5,7-Di-m-tolyl-7H-pyrrolo[2,3-d]pyrimidin-4-yl)-(2-methoxy-ethyl)-amine;
(2-Methoxy-ethyl)-[5-(4-methoxy-phenyl)-7-m-tolyl-7H-pyrrolo[2,3-d]pyrimidin-4-yl]-amine;
(S)-2-[7-(3-Chloro-4-methyl-phenyl)-5-phenyl-7H-pyrrolo[2,3-d]pyrimidin-4-ylamino]-propan-1-ol;
4-[4-(Tetrahydro-pyran-4-ylamino)-7-m-tolyl-7H-pyrrolo[2,3-d]pyrimidin-5-yl]-phenol;
[5-(2-Chloro-phenyl)-7-m-tolyl-7H-pyrrolo[2,3-d]pyrimidin-4-yl]-(tetrahydro-pyran-4-yl)-amine;
[5-(3-Methoxy-phenyl)-7-m-tolyl-7H-pyrrolo[2,3-d]pyrimidin-4-yl]-(tetrahydro-pyran-4-yl)-amine;
[7-(3-Ethoxy-phenyl)-5-phenyl-7H-pyrrolo[2,3-d]pyrimidin-4-yl]-(2-methoxy-ethyl)-amine;
2-[5-(4-Fluoro-phenyl)-7-m-tolyl-7H-pyrrolo[2,3-d]pyrimidin-4-ylamino]-2-hydroxymethyl-propane-1,3-diol;
4-[7-(3-Chloro-4-methyl-phenyl)-5-phenyl-7H-pyrrolo[2,3-d]pyrimidin-4-ylamino]-cyclo hexanol;
2-[5-(4-Fluoro-phenyl)-7-m-tolyl-7H-pyrrolo[2,3-d]pyrimidin-4-ylamino]-2-methyl-propan-1-ol;
2-[5-(4-Fluoro-phenyl)-7-m-tolyl-7H-pyrrolo[2,3-d]pyrimidin-4-ylamino]-2-methyl-propane-1,3-diol; compound with trifluoro-acetic acid;
[5-(3,5-Difluoro-phenyl)-7-m-tolyl-7H-pyrrolo[2,3-d]pyrimidin-4-yl]-(tetrahydro-pyran-4-yl)-amine;
(2-Methoxy-ethyl)-(7-m-tolyl-5-o-tolyl-7H-pyrrolo[2,3-d]pyrimidin-4-yl)-amine;
(2-Methoxy-ethyl)-[7-(3-methoxy-phenyl)-5-phenyl-7H-pyrrolo[2,3-d]pyrimidin-4-yl]-amine;
(Tetrahydro-pyran-4-yl)-(7-m-tolyl-5-p-tolyl-7H-pyrrolo[2,3-d]pyrimidin-4-yl)-amine;
[5-(3-Isopropoxy-phenyl)-7-m-tolyl-7H-pyrrolo[2,3-d]pyrimidin-4-yl]-(tetrahydro-pyran-4-yl)-amine;
[5-(4-Chloro-phenyl)-7-m-tolyl-7H-pyrrolo[2,3-d]pyrimidin-4-yl]-(2-methoxy-ethyl)-amine;
[5-(3,4-Dimethyl-phenyl)-7-m-tolyl-7H-pyrrolo[2,3-d]pyrimidin-4-yl]-(tetrahydro-pyran-4-yl)-amine;
[5-(3-Fluoro -5-methyl-phenyl)-7-m-tolyl-7H-pyrrolo[2,3-d]pyrimidin-4-yl]-(tetrahydro-pyran-4-yl)-amine;
[5-(1H-Pyrrol-2-yl)-7-m-tolyl-7H-pyrrolo[2,3-d]pyrimidin-4-yl]-(tetrahydro-pyran-4yl)-amine;
[7-(3-tert-Butyl-phenyl)-5-phenyl-7H-pyrrolo[2,3-d]pyrimidin-4-yl]-(2-methoxy-ethyl)-amine;
(5-Methyl-7-m-tolyl-7H-pyrrolo[2,3-d]pyrimidin-4-yl)-(tetrahydro-pyran-4-yl)-amine;
[7-(3-Fluoro-phenyl)-5-phenyl-7H-pyrrolo[2,3-d]pyrimidin-4-yl]-(2-methoxy-ethyl)-amine;
[5-(4-Fluoro-phenyl)-7-m-tolyl-7H-pyrrolo[2,3-d]pyrimidin-4-yl]-(1-methyl-piperidin-4-yl)-amine;
[5-(4-Chloro-phenyl)-7-m-tolyl-7H-pyrrolo[2,3-d]pyrimidin-4-yl]-(2-methoxy-ethyl)-amine;
[5-(2,4-Difluoro-phenyl)-7-m-tolyl-7H-pyrrolo[2,3-d]pyrimidin-4-yl]-(tetrahydro-pyran-4-yl)-amine;
N-{4-[4-(Tetrahydro-pyran-4-ylamino)-7-m-tolyl-7H-pyrrolo[2,3-d]pyrimidin-5-yl]-phenyl}-acetamide;
[5-(3-Methoxy-phenyl)-7-m-tolyl-7H-pyrrolo[2,3-d]pyrimidin-4-yl]-(tetrahydro-pyran-4-yl)-amine;
(5-Pyridin-3-yl-7-m-tolyl-7H-pyrrolo[2,3-d]pyrimidin-4-yl)-(tetrahydro-pyran-4-yl)-amine;
[7-(3,5-Dichloro-phenyl)-5-phenyl-7H-pyrrolo[2,3-d]pyrimidin-4-yl]-(2-methoxy-ethyl)-amine;
[7-(4-Chloro-3-methyl-phenyl)-5-phenyl-7H-pyrrolo[2,3-d]pyrimidin-4-yl]-(2-methoxy-ethyl)-amine;
[5-(3-Chloro-5-fluoro-phenyl)-7-m-tolyl-7H-pyrrolo[2,3-d]pyrimidin-4-yl]-piperidin-4-yl-amine;
[5-(4-Fluoro-phenyl)-7-m-tolyl-7H-pyrrolo[2,3-d]pyrimidin-4-yl]-piperidin-4-yl-amine;
N-{4-[4-(Tetrahydro-pyran-4-ylamino)-7-m-tolyl-7H-pyrrolo[2,3-d]pyrimidin-5-yl]-phenyl}-methanesulfonamide;
(2-Methoxy-ethyl)-[5-phenyl-7-(4-trifluoromethyl-phenyl)-7H-pyrrolo[2,3-d]pyrimidin-4-yl]-amine;
[5-(4-Chloro-phenyl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(4-trifluoromethyl-phenyl)-7H-pyrrolo[2,3-d]pyrimidin-4-yl]-amine;
(2-Methoxy-ethyl)-[7-(4-methoxy-3-methyl-phenyl)-5-phenyl-7H-pyrrolo[2,3-d]pyrimidin-4-yl]-amine;
[5-(4-Fluoro-phenyl)-7-m-tolyl-7H-pyrrolo[2,3-d]pyrimidin-4-yl]-(2-methoxy-ethyl)-amine;
(Tetrahydro-pyran-4-yl)-(7-m-tolyl-5-o-tolyl-7H-pyrrolo[2,3-d]pyrimidin-4-yl)-amine;
4-[4-(Tetrahydro-pyran-4-ylamino)-7-m-tolyl-7H-pyrrolo[2,3-d]pyrimidin-5-yl]-benzamide;
[7-(4-Fluoro-3-methyl-phenyl)-5-phenyl-7H-pyrrolo[2,3-d]pyrimidin-4-yl]-(2-methoxy-ethyl)-amine;
2-Chloro-N,N-dimethyl-4-[4-(tetrahydro-pyran-4-ylamino)-7-m-tolyl-7H-pyrrolo[2,3-d]pyrimidin-5-yl]-benzamide;
7-(3-Chloro-phenyl)-4-(4-methyl-piperazin-1-yl)-5-phenyl-7H-pyrrolo[2,3-d]pyrimidine;
[5-(3,4-Dimethyl-phenyl)-7-m-tolyl-7H-pyrrolo[2,3-d]pyrimidin-4-yl]-(tetrahydro-pyran-4-yl)-amine;
2-[4-(5-Phenyl-7-m-tolyl-7H-pyrrolo[2,3-d]pyrimidin-4-yl)-piperazin-1-yl]-ethanol;
N-{3-[4-(Tetrahydro-pyran-4-ylamino)-7-m-tolyl-7H-pyrrolo[2,3-d]pyrimidin-5-yl]-phenyl}-acetamide;
[5-(2-Methylsulfanyl-phenyl)-7-m-tolyl-7H-pyrrolo[2,3-d]pyrimidin-4-yl]-(tetrahydro-pyran-4-yl)-amine;
[5-(4-Methoxy-3-trifluoromethyl-phenyl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(4-trifluoromethyl-phenyl)-7H-pyrrolo[2,3-d]pyrimidin-4-yl]-amine;
[7-(3,4-Dimethoxy-phenyl)-5-phenyl-7H-pyrrolo[2,3-d]pyrimidin-4-yl]-(2-methoxy-ethyl)-amine;
(2-Methoxy-ethyl)-[7-(4-morpholin-4-yl-phenyl)-5-phenyl-7H-pyrrolo[2,3-d]pyrimidin-4-yl]-amine;
[5-(2-Chloro-phenyl)-7-m-tolyl-7H-pyrrolo[2,3-d]pyrimidin-4-yl]-(tetrahydro-pyran-4-yl)-amine;
2-{4-[7-(3-Chloro-phenyl)-5-phenyl-7H-pyrrolo[2,3-d]pyrimidin-4-yl]-piperazin-1-yl}-ethanol;
[7-(2-Fluoro-phenyl)-5-phenyl-7H-pyrrolo[2,3-d]pyrimidin-4-yl]-(2-methoxy-ethyl)-amine;
[7-(3-Chloro-4-methyl-phenyl)-5-phenyl-7H-pyrrolo[2,3-d]pyrimidin-4-yl]-(2-methoxy-1-methoxymethyl-ethyl)-amine;
[7-(3-Chloro-4-methyl-phenyl)-5-phenyl-7H-pyrrolo[2,3-d]pyrimidin-4-yl]-(2-methoxy-1-methoxymethyl-ethyl)-amine;
[7-(3-Chloro-4-methyl-phenyl)-5-phenyl-7H-pyrrolo[2,3-d]pyrimidin-4-yl]-(2-pyridin-3-yl-ethyl)-amine;
[7-(3-Chloro-4-methyl-phenyl)-5-phenyl-7H-pyrrolo[2,3-d]pyrimidin-4-yl]-(2-pyridin-2-yl-ethyl)-amine;
2-Chloro-4-[4-(tetrahydro-pyran-4-ylamino)-7-m-tolyl-7H-pyrrolo[2,3-d]pyrimidin-5-yl]-benzamide
[7-(3-Chloro-4-methyl-phenyl)-5-phenyl-7H-pyrrolo[2,3-d]pyrimidin-4-yl]-(2-imidazol-1-yl-ethyl)-amine;
[7-(3-Fluoro-4-methyl-phenyl)-5-phenyl-7H-pyrrolo[2,3-d]pyrimidin-4-yl]-(2-methoxy-ethyl)-amine;
4-(4-Methyl-piperazin-1-yl)-5-phenyl-7-p-tolyl-7H-pyrrolo[2,3-d]pyrimidine;
{2-Chloro-4-[4-(tetrahydro-pyran-4-ylamino)-7-m-tolyl-7H-pyrrolo[2,3-d]pyrimidin-5-yl]-phenyl}-pyrrolidin-1-yl-methanone;
{2-Chloro-4-[4-(tetrahydro-pyran-4-ylamino)-7-m-tolyl-7H-pyrrolo[2,3-d]pyrimidin-5-yl]-phenyl}-morpholin-4-yl-methanone;
2-Fluoro-4-[4-(tetrahydro-pyran-4-ylamino)-7-m-tolyl-7H-pyrrolo[2,3-d]pyrimidin-5-yl]-benzoic acid methyl ester;
[5-(5-Chloro-2-methyl-phenyl)-7-m-tolyl-7H-pyrrolo[2,3-d]pyrimidin-4-yl]-(tetrahydro-pyran-4-yl)-amine;
[5-(2,3-Difluoro-phenyl)-7-m-tolyl-7H-pyrrolo[2,3-d]pyrimidin-4-yl]-(tetrahydro-pyran-4-yl)-amine;
[5-(3-Fluoro-phenyl)-7-m-tolyl-7H-pyrrolo[2,3-d]pyrimidin-4-yl]-(tetrahydro-pyran-4-yl)-amine;
[5-(3-Ethoxy-phenyl)-7-m-tolyl-7H-pyrrolo[2,3-d]pyrimidin-4-yl]-(tetrahydro-pyran-4-yl)-amine;
[5-(4-Methylsulfanyl-phenyl)-7-m-tolyl-7H-pyrrolo[2,3-d]pyrimidin-4-yl]-(tetrahydro-pyran-4-yl)-amine;
[5-(2-Ethoxy-phenyl)-7-m-tolyl-7H-pyrrolo[2,3-d]pyrimidin-4-yl]-(tetrahydro-pyran-4-yl)-amine;
[5-(3,5-Dimethyl-phenyl)-7-m-tolyl-7H-pyrrolo[2,3-d]pyrimidin-4-yl]-(tetrahydro-pyran-4-yl)-amine;
[5-(4-Fluoro-3-trifluoromethyl-phenyl)-7-m-tolyl-7H-pyrrolo[2,3-d]pyrimidin-4-yl]-(tetrahydro-pyran-4-yl)-amine;
[5-(3-Fluoro-4-methoxy-phenyl)-7-m-tolyl-7H-pyrrolo[2,3-d]pyrimidin-4-yl]-(tetrahydro-pyran-4-yl)-amine;
[5-(3-Fluoro-5-isopropoxy-phenyl)-7-m-tolyl-7H-pyrrolo[2,3-d]pyrimidin-4-yl]-(tetrahydro-pyran-4-yl)-amine;
[5-(3-Ethoxy-5-fluoro-phenyl)-7-m-tolyl-7H-pyrrolo[2,3-d]pyrimidin-4-yl]-(tetrahydro-pyran-4-yl)-amine;
[5-(3-Methylsulfanyl-phenyl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(3-trifluoromethyl-phenyl)-7H-pyrrolo[2,3-d]pyrimidin-4-yl]-amine;
(Tetrahydro-pyran-4-yl)-[7-m-tolyl-5-(3-trifluoromethyl-phenyl)-7H-pyrrolo[2,3-d]pyrimidin-4-yl]-amine;
[5-(4-tert-Butyl-phenyl)-7-m-tolyl-7H-pyrrolo[2,3-d]pyrimidin-4-yl]-(tetrahydro-pyran-4-yl)-amine;
[5-(2-Ethyl-phenyl)-7-m-tolyl-7H-pyrrolo[2,3-d]pyrimidin-4-yl]-(tetrahydro-pyran-4-yl)-amine;
[5-(2-Ethyl-phenyl)-7-m-tolyl-7H-pyrrolo[2,3-d]pyrimidin-4-yl]-(tetrahydro-pyran-4yl)-amine;
[5-(2-Fluoro-phenyl)-7-m-tolyl-7H-pyrrolo[2,3-d]pyrimidin-4-yl]-(tetrahydro-pyran-4-yl)-amine;
[5-(2-Methoxy-phenyl)-7-m-tolyl-7H-pyrrolo[2,3-d]pyrimidin-4-yl]-(tetrahydro-pyran-4-yl)-amine;
[5-(4-Ethyl-phenyl)-7-m-tolyl-7H-pyrrolo[2,3-d]pyrimidin-4-yl]-(tetrahydro-pyran-4-yl)-amine;
3-[4-(Tetrahydro-pyran-4-ylamino)-7-m-tolyl-7H-pyrrolo[2,3-d]pyrimidin-5-yl]-benzonitrile;
[5-(4-Ethoxy-phenyl)-7-m-tolyl-7H-pyrrolo[2,3-d]pyrimidin-4-yl]-(tetrahydro-pyran-4-yl)-amine;
[5-(3-Chloro-phenyl)-7-m-tolyl-7H-pyrrolo[2,3-d]pyrimidin-4-yl]-(tetrahydro-pyran-4-yl)-amine;
[5-(3,4-Dimethoxy-phenyl)-7-m-tolyl-7H-pyrrolo[2,3-d]pyrimidin-4-yl]-(tetrahydro-pyran-4-yl)-amine;
[5-(3-Isopropyl-phenyl)-7-m-tolyl-7H-pyrrolo[2,3-d]pyrimidin-4-yl]-(tetrahydro-pyran-4-yl)-amine;
[5-(4-Isopropyl-phenyl)-7-m-tolyl-7H-pyrrolo[2,3-d]pyrimidin-4-yl]-(tetrahydro-pyran-4-yl)-amine;
(5,7-Di-m-tolyl-7H-pyrrolo[2,3-d]pyrimidin-4-yl)-(tetrahydro-pyran-4-yl)-amine
[5-(4-Methoxy-phenyl)-7-m-tolyl-7H-pyrrolo[2,3-d]pyrimidin-4-yl]-(tetrahydro-pyran-4-yl)-amine;
[5-(2-Isopropoxy-phenyl)-7-m-tolyl-7H-pyrrolo[2,3-d]pyrimidin-4-yl]-(tetrahydro-pyran-4-yl)-amine;
(5-Ethyl-7-m-tolyl-7H-pyrrolo[2,3-d]pyrimidin-4-yl)-(tetrahydro-pyran-4-yl)-amine;
(Tetrahydro-pyran-4-yl)-[7-m-tolyl-5-(2-p-tolyl- ethyl)-7H-pyrrolo[2,3-d]pyrimidin-4-yl]-amine;
(5-Butyl-7-m-tolyl-7H-pyrrolo[2,3-d]pyrimidin-4-yl)-(tetrahydro-pyran-4-yl)-amine;
(Tetrahydro-pyran-4-yl)-[7-m-tolyl-5-(3,4,5-trimethoxy-phenyl)-7H-pyrrolo[2,3-d]pyrimidin-4-yl]-amine
[5-(4-Methanesulfonyl-phenyl)-7-m-tolyl-7H-pyrrolo[2,3-d]pyrimidin-4-yl]-(tetrahydro-pyran-4-yl)-amine;
1-{4-[4-(Tetrahydro-pyran-4-ylamino)-7-m-tolyl-7H-pyrrolo[2,3-d]pyrimidin-5-yl]-phenyl}-ethanone;
1-{2-[4-(Tetrahydro-pyran-4-ylamino)-7-m-tolyl-7H-pyrrolo[2,3-d]pyrimidin-5-yl]-phenyl}-ethanone;
4-[4-(Tetrahydro-pyran-4-ylamino)-7-m-tolyl-7H-pyrrolo[2,3-d]pyrimidin-5-yl]-benzoic acid ethyl ester;
(Tetrahydro-pyran-4-yl)-(5-thiophen-2-yl-7-m-tolyl-7H-pyrrolo[2,3-d]pyrimidin-4-yl)-amine;
N-{3-[4-(Tetrahydro-pyran-4-ylamino)-7-m-tolyl-7H-pyrrolo[2,3-d]pyrimidin-5-yl]-phenyl}-methanesulfonamide;
(5-Quinolin-8-yl-7-m-tolyl-7H-pyrrolo[2,3-d]pyrimidin-4-yl)-(tetrahydro-pyran-4-yl)-amine;
N-Methyl-4-[4-(tetrahydro-pyran-4-ylamino)-7-m-tolyl-7H-pyrrolo[2,3-d]pyrimidin-5-yl]-benzenesulfonamide;
[5-(3-Amino-phenyl)-7-m-tolyl-7H-pyrrolo[2,3-d]pyrimidin-4-yl]-(tetrahydro-pyran-4-yl)-amine;
N,N-Diethyl-2-[4-(tetrahydro-pyran-4-ylamino)-7-m-tolyl-7 H-pyrrolo[2,3-d]pyrimidin-5-yl]-benzamide;
[5-((E)-Pent-1-enyl)-7-m-tolyl-7H-pyrrolo[2,3-d]pyrimidin-4-yl]-(tetrahydro-pyran-4-yl)-amine;
[5-(1H-Pyrrol-3-yl)-7-m-tolyl-7H-pyrrolo[2,3-d]pyrimidin-4-yl]-(tetrahydro-pyran-4-yl)-amine;
[5-(1-Methyl-1H-pyrrol-2-yl)-7-m-tolyl-7H-pyrrolo[2,3-d]pyrimidin-4-yl]-(tetrahydro-pyran-4-yl)-amine;
(Tetrahydro-pyran-4-yl)-[7-m-tolyl-5-(2-vinyl-phenyl)-7H-pyrrolo[2,3-d]pyrimidin-4-yl]-amine;
N-tert-Butyl-3-[4-(tetrahydro-pyran-4-ylamino)-7-m-tolyl-7H-pyrrolo[2,3-d]pyrimidin-5-yl]-benzenesulfonamide;
(5-Cyclopent-1-enyl-7-m-tolyl-7H-pyrrolo[2,3-d]pyrimidin-4-yl)-(tetrahydro-pyran-4-yl)-amine;
(5-Cyclohex-1-enyl-7-m-tolyl-7H-pyrrolo[2,3-d]pyrimidin-4-yl)-(tetrahydro-pyran-4-yl)-amine;
3-[4-(Tetrahydro-pyran-4-ylamino)-7-m-tolyl-7H-pyrrolo[2,3-d]pyrimidin-5-yl]-benzenesulfonamide;
[5-(4-Dimethylamino-phenyl)-7-m-tolyl-7H-pyrrolo[2,3-d]pyrimidin-4-yl]-(tetrahydro-pyran-4-yl)-amine;
[5-(3-Chloro-4-fluoro-phenyl)-7-m-tolyl-7H-pyrrolo[2,3-d]pyrimidin-4-yl]-(tetrahydro-pyran-4-yl)-amine;
(Tetrahydro-pyran-4-yl)-(5-thiophen-3-yl-7-m-tolyl-7H-pyrrolo[2,3-d]pyrimidin-4-yl)-amine;
1-{5-[4-(Tetrahydro-pyran-4-ylamino)-7-m-tolyl-7H-pyrrolo[2,3-d]pyrimidin-5-yl]-thiophen-2-yl}-ethanone;
[5-(4-Methyl-thiophen-2-yl)-7-m-tolyl-7H-pyrrolo[2,3-d]pyrimidin-4-yl]-(tetrahydro-pyran-4-yl)-amine;
[5-(3,4-Difluoro-phenyl)-7-m-tolyl-7H-pyrrolo[2,3-d]pyrimidin-4-yl]-(tetrahydro-pyran-4-yl)-amine;
(5-Phenyl-7-m-tolyl-7H-pyrrolo[2,3-d]pyrimidin-4-yl)-(tetrahydro-pyran-4-yl)-amine;
(5-Phenyl-7-m-tolyl-7H-pyrrolo[2,3-d]pyrimidin-4-yl)-[2-(tetrahydro-pyran-4-yl)-ethyl]-amine;
(5-Phenyl-7-m-tolyl-7H-pyrrolo[2,3-d]pyrimidin-4-yl)-(tetrahydro-pyran-4-ylmethyl)-amine;
(5,7-Diphenyl-7H-pyrrolo[2,3-d]pyrimidin-4-yl)-(tetrahydro-pyran-4-yl)-amine;
(5,7-Diphenyl-7H-pyrrolo[2,3-d]pyrimidin-4-yl)-(tetrahydro-pyran-4-ylmethyl)-amine;
[5-(1-Methyl-1H-pyrazol-4-yl)-7-m-tolyl-7H-pyrrolo[2,3-d]pyrimidin-4-yl]-(tetrahydro-pyran-4-yl)-amine;
[5-(1H-Pyrazol-3-yl)-7-m-tolyl-7H-pyrrolo[2,3-d]pyrimidin-4-yl]-(tetrahydro-pyran-4-yl)-amine;
[5-(1H-Pyrazol-4-yl)-7-m-tolyl-7H-pyrrolo[2,3-d]pyrimidin-4-yl]-(tetrahydro-pyran-4-yl)-amine;
(S)-2-[5-(2-Methyl-propenyl)-7-p-tolyl-7H-pyrrolo[2,3-d]pyrimidin-4-ylamino]-propan-1-ol; and
(S)-2-(5-Isobutyl-7-p-tolyl-7H-pyrrolo[2,3-d]pyrimidin-4-ylamino)-propan-1-ol.

21. A pharmaceutical composition comprising:
(a) a pharmaceutically acceptable carrier; and
(b) the compound of claim 1.

22. A method for treating a pain condition selected from inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injury, or pain associated with irritable bowel syndrome, said method comprising administering to a subject in need thereof an effective amount of the compound of claim 1.

23. A method for treating a respiratory disorder selected from chronic obstructive pulmonary disorder (COPD), asthma, and bronchospasm, said method comprising administering to a subject in need thereof an effective amount of the compound of claim 1.
